SEQUENCE LISTING (118 sequences)

SEQ ID NO: 1 - protein, 17 aa, synthetic construct
ARTKQTARKSTGGKAPR

SEQ ID NO: 2 - protein, 17 aa, synthetic construct
TARKSTGGKAPRKQLAT

SEQ ID NO: 3 - protein, 17 aa, synthetic construct
KSTGGKAPRKQLATKAA

SEQ ID NO: 4 - protein, 19 aa, synthetic construct
KQLATKAARKSAPATGGVK

SEQ ID NO: 5 - protein, 19 aa, synthetic construct
KSAPSTGGVKKPHRYKPGT

SEQ ID NO: 6 - protein, 15 aa, synthetic construct
GGAKRHRKVLRDNIQ

SEQ ID NO: 7 - protein, 17 aa, synthetic construct
  SITE 9: note = Mono-methylated residue
ARTKQTARKSTGGKAPR

SEQ ID NO: 8 - protein, 17 aa, synthetic construct
  SITE 9: note = Di-methylated residue
ARTKQTARKSTGGKAPR

SEQ ID NO: 9 - protein, 17 aa, synthetic construct
  SITE 9: note = Tri-methylated residue
ARTKQTARKSTGGKAPR

SEQ ID NO: 10 - protein, 17 aa, synthetic construct
  SITE 9: note = Mono-methylated residue
KSTGGKAPRKQLATKAA

SEQ ID NO: 11 - protein, 19 aa, synthetic construct
  SITE 10: note = Mono-methylated residue
KQLATKAARKSAPATGGVK

SEQ ID NO: 12 - protein, 19 aa, synthetic construct
  SITE 10: note = Di-methylated residue
KQLATKAARKSAPATGGVK

SEQ ID NO: 13 - protein, 19 aa, synthetic construct
  SITE 10: note = Tri-methylated residue
KQLATKAARKSAPATGGVK

SEQ ID NO: 14 - protein, 19 aa, synthetic construct
  SITE 10: note = Tri-methylated residue
KSAPSTGGVKKPHRYKPGT

SEQ ID NO: 15 - protein, 15 aa, synthetic construct
  SITE 8: note = Tri-methylated residue
GGAKRHRKVLRDNIQ

SEQ ID NO: 16 - protein, 17 aa, synthetic construct
  SITE 9: note = Acetylated residue
TARKSTGGKAPRKQLAT

SEQ ID NO: 17 - protein, 17 aa, synthetic construct
  SITE 9: note = Acetylated residue
ARTKQTARKSTGGKAPR

SEQ ID NO: 18 - protein, 19 aa, synthetic construct
  SITE 10: note = Acetylated residue
KQLATKAARKSAPATGGVK

SEQ ID NO: 19 - DNA, 53 bp, synthetic construct
  source 1..53: mol_type = other DNA
ggcagcagcgcaattnnnnnnnnnnnnnnngttgaaagcattcgtaaaaaacg

SEQ ID NO: 20 - DNA, 84 bp, synthetic construct
  source 1..84: mol_type = other DNA
cgtaaaggtaaagttgaatatctggttaaanngnnannnnngnngnngnnannnnnnnnntgggaaccggaagaacatattctg

SEQ ID NO: 21 - protein, 63 aa, Homo sapiens
EEEEYVVEKVLDRRVVKGKVEYLLKWKGFSDEDNTWEPEENLDCPDLIAEFLQSQKTAHETDK

SEQ ID NO: 22 - protein, 63 aa, Homo sapiens
GEQVFAAECILSKRLRKGKLEYLVKWRGWSSKHNSWEPEENILDPRLLLAFQKKEHEKEVQNR

SEQ ID NO: 23 - protein, 59 aa, Homo sapiens
EEAEPEEFVVEKVLDRRVVNGKVEYFLKWKGFTDADNTWEPEENLDCPELIEAFLNSQK

SEQ ID NO: 24 - protein, 64 aa, Homo sapiens
VGEHVFAVESIEKKRIRKGRVEYLVKWRGWSPKYNTWEPEENILDPRLLIAFQNRERQEQLMGY

SEQ ID NO: 25 - protein, 62 aa, Homo sapiens
EEEYVVEKVLDRRVVKGQVEYLLKWKGFSEEHNTWEPEKNLDCPELISEFMKKYKKMKEGEN

SEQ ID NO: 26 - protein, 64 aa, Homo sapiens
VGERVFAAESIIKRRIRKGRIEYLVKWKGWAIKYSTWEPEENILDSRLIAAFEQKERERELYGP

SEQ ID NO: 27 - protein, 72 aa, Homo sapiens
SAIGEQVFAVESIRKKRVRKGKVEYLVKWKGWPPKYSTWEPEEHILDPRLVMAYEEKEERDRASGYRKRGPK

SEQ ID NO: 28 - protein, 63 aa, Homo sapiens
GERVFAAEALLKRRIRKGRMEYLVKWKGWSQKYSTWEPEENILDARLLAAFEEREREMELYGP

SEQ ID NO: 29 - protein, 63 aa, synthetic construct
EEDEYVVEKVLDRRVVKGKVEYLLKWEGFSDEDNTWEPEENLDCPDLIAEFLQSQKTAHETDK

SEQ ID NO: 30 - protein, 63 aa, synthetic construct
GEDVFAAECILSKRLRKGKLEYLVKWEGWSSKHNSWEPEENILDPRLLLAFQKKEHEKEVQNR

SEQ ID NO: 31 - protein, 59 aa, synthetic construct; identical to SEQ ID NO: 23

SEQ ID NO: 32 - protein, 63 aa, synthetic construct
GEDVFAVESIEKKRIRKGRVEYLVKWEGWSPKYNTWEPEENILDPRLLIAFQNRERQEQLMGY

SEQ ID NO: 33 - protein, 62 aa, synthetic construct
EDEYVVEKVLDRRVVKGQVEYLLKWEGFSEEHNTWEPEKNLDCPELISEFMKKYKKMKEGEN

SEQ ID NO: 34 - protein, 64 aa, synthetic construct
VGDRVFAAESIIKRRIRKGRIEYLVKWEGWAIKYSTWEPEENILDSRLIAAFEQKERERELYGP

SEQ ID NO: 35 - protein, 72 aa, synthetic construct
SAIGEDVFAVESIRKKRVRKGKVEYLVKWEGWPPKYSTWEPEEHILDPRLVMAYEEKEERDRASGYRKRGPK

SEQ ID NO: 36 - protein, 63 aa, synthetic construct
GEDVFAAEALLKRRIRKGRMEYLVKWEGWSQKYSTWEPEENILDARLLAAFEEREREMELYGP

SEQ ID NO: 37 - protein, 73 aa, synthetic construct
LSSVGEDVFAAECILSKRLRKGKLEYLVKWEGWSSKHNSWEPEENILDPRLLLAFQKKEHEKEVQNRKRGKRP

SEQ ID NO: 38 - protein, 59 aa, synthetic construct; identical to SEQ ID NO: 23

SEQ ID NO: 39 - protein, 62 aa, synthetic construct; identical to SEQ ID NO: 33

SEQ ID NO: 40 - protein, 75 aa, synthetic construct
ELSAIGEDVFAVESIRKKRVRKGKVEYLVKWEGWPPKYSTWEPEEHILDPRLVMAYEEKEERDRASGYRKRGPKP

SEQ ID NO: 41 - protein, 97 aa, synthetic construct
MSYYHHHHHHGSDYKDDDDKSSGENLYFQGSGGSEEEEYVVEKVLDRRVVKGKVEYLLKWKGFSDEDNTWEPEENLDCPDLIAEFLQSQKTAHETDK

SEQ ID NO: 42 - protein, 97 aa, synthetic construct
MSYYHHHHHHGSDYKDDDDKSSGENLYFQGSGGSGEQVFAAECILSKRLRKGKLEYLVKWKGWSSKHNSWEPEENILDPRLLLAFQKKEHEKEVQNR

SEQ ID NO: 43 - protein, 93 aa, synthetic construct
MSYYHHHHHHGSDYKDDDDKSSGENLYFQGSGGSEEAEPEEFVVEKVLDRRVVNGKVEYFLKWKGFTDADNTWEPEENLDCPELIEAFLNSQK

SEQ ID NO: 44 - protein, 97 aa, synthetic construct
MSYYHHHHHHGSDYKDDDDKSSGENLYFQGSGGSGEHVFAVESIEKKRIRKGRVEYLVKWRGWSPKYNTWEPEENILDPRLLIAFQNRERQEQLMGY

SEQ ID NO: 45 - protein, 96 aa, synthetic construct
MSYYHHHHHHGSDYKDDDDKSSGENLYFQGSGGSEEEYVVEKVLDRRVVKGQVEYLLKWKGFSEEHNTWEPEKNLDCPELISEFMKKYKKMKEGEN

SEQ ID NO: 46 - protein, 97 aa, synthetic construct
MSYYHHHHHHGSDYKDDDDKSSGENLYFQGSGGSVGERFAAESIIKRRIRKGRIEYLVKWKGWAIKYSTWEPEENILDSRLIAAFEQKERERELYGP

SEQ ID NO: 47 - protein, 106 aa, synthetic construct
MSYYHHHHHHGSDYKDDDDKSSGENLYFQGSGGSSAIGEQVFAVESIRKKRVRKGKVEYLVKWKGWPPKYSTWEPEEHILDPRLVMAYEEKEERDRASGYRKRGPK

SEQ ID NO: 48 - protein, 97 aa, synthetic construct
MSYYHHHHHHGSDYKDDDDKSSGENLYFQGSGGSGERVFAAEALLKRRIRKGRMEYLVKWKGWSQKYSTWEPEENILDARLLAAFEEREREMELYGP

SEQ ID NO: 49 - protein, 97 aa, synthetic construct
MSYYHHHHHHGSDYKDDDDKSSGENLYFQGSGGSEEDEYVVEKVLDRRVVKGKVEYLLKWEGFSDEDNTWEPEENLDCPDLIAEFLQSQKTAHETDK

SEQ ID NO: 50 - protein, 97 aa, synthetic construct
MSYYHHHHHHGSDYKDDDDKSSGENLYFQGSGGSGEDVFAAECILSKRLRKGKLEYLVKWEGWSSKHNSWEPEENILDPRLLLAFQKKEHEKEVQNR

SEQ ID NO: 51 - protein, 93 aa, synthetic construct; identical to SEQ ID NO: 43

SEQ ID NO: 52 - protein, 97 aa, synthetic construct
MSYYHHHHHHGSDYKDDDDKSSGENLYFQGSGGSGEDVFAVESIEKKRIRKGRVEYLVKWEGWSPKYNTWEPEENILDPRLLIAFQNRERQEQLMGY

SEQ ID NO: 53 - protein, 96 aa, synthetic construct
MSYYHHHHHHGSDYKDDDDKSSGENLYFQGSGGSEDEYVVEKVLDRRVVKGQVEYLLKWEGFSEEHNTWEPEKNLDCPELISEFMKKYKKMKEGEN

SEQ ID NO: 54 - protein, 98 aa, synthetic construct
MSYYHHHHHHGSDYKDDDDKSSGENLYFQGSGGSVGDRVFAAESIIKRRIRKGRIEYLVKWEGWAIKYSTWEPEENILDSRLIAAFEQKERERELYGP

SEQ ID NO: 55 - protein, 106 aa, synthetic construct
MSYYHHHHHHGSDYKDDDDKSSGENLYFQGSGGSSAIGEDVFAVESIRKKRVRKGKVEYLVKWEGWPPKYSTWEPEEHILDPRLVMAYEEKEERDRASGYRKRGPK

SEQ ID NO: 56 - protein, 97 aa, synthetic construct
MSYYHHHHHHGSDYKDDDDKSSGENLYFQGSGGSGEDVFAAEALLKRRIRKGRMEYLVKWEGWSQKYSTWEPEENILDARLLAAFEEREREMELYGP

SEQ ID NO: 57 - protein, 376 aa, synthetic construct
MAGGLNDIFEAQKIEWHENLYFQGVDEESGSVSGGDLSSVGEDVFAAECILSKRLRKGKLEYLVKWEGWSSKHNSWEPEENILDPRLLLAFQKKEHEKEVQNRKRGKRPIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 58 - protein, 479 aa, synthetic construct
MAGGLNDIFEAQKIEWHENLYFQGVDEESGSVSGGDLSSVGEDVFAAECILSKRLRKGKLEYLVKWEGWSSKHNSWEPEENILDPRLLLAFQKKEHEKEVQNRKRGKRPGGGGSEGGGSGGPGSGGEGSAGGGSAGGGSLSSVGEDVFAAECILSKRLRKGKLEYLVKWEGWSSKHNSWEPEENILDPRLLLAFQKKEHEKEVQNRKRGKRPIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 59 - protein, 378 aa, synthetic construct
MAGGLNDIFEAQKIEWHENLYFQGVDEESGSVSGGDELSAIGEDVFAVESIRKKRVRKGKVEYLVKWEGWPPKYSTWEPEEHILDPRLVMAYEEKEERDRASGYRKRGPKPIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 60 - protein, 483 aa, synthetic construct
MAGGLNDIFEAQKIEWHENLYFQGVDEESGSVSGGDELSAIGEDVFAVESIRKKRVRKGKVEYLVKWEGWPPKYSTWEPEEHILDPRLVMAYEEKEERDRASGYRKRGPKPGGGGSEGGGSGGPGSGGEGSAGGGSAGGGSELSAIGEDVFAVESIRKKRVRKGKVEYLVKWEGWPPKYSTWEPEEHILDPRLVMAYEEKEERDRASGYRKRGPKPIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 61 - protein, 362 aa, synthetic construct
MAGGLNDIFEAQKIEWHENLYFQGVDEESGSVSGGDEEAEPEEFVVEKVLDRRVVNGKVEYFLKWKGFTDADNTWEPEENLDCPELIEAFLNSQKIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 62 - protein, 365 aa, synthetic construct
MAGGLNDIFEAQKIEWHENLYFQGVDEESGSVSGGDEDEYVVEKVLDRRVVKGQVEYLLKWEGFSEEHNTWEPEKNLDCPELISEFMKKYKKMKEGENIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 63 - protein, 362 aa, synthetic construct; identical to SEQ ID NO: 61

SEQ ID NO: 64 - protein, 365 aa, synthetic construct
MAGGLNDIFEAQKIEWHENLYFQGVDEESGSVSGGDEEEYVVEKVLDRRVVKGQVEYLLKWKGFSEEHNTWEPEKNLDCPELISEFMKKYKKMKEGENIGGGGSGGGGSAPPKKKRKVGYRDPPVATVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 65 - protein, 75 aa, synthetic construct
MDAKSLTAWSRTLVTFKDVFVDFTREEWKLLDTAQQIVYRNVMLENYKNLVSLGYQLTKPDVILRLEKGEEPWLV

SEQ ID NO: 66 - protein, 101 aa, synthetic construct
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLS

SEQ ID NO: 67 - protein, 159 aa, synthetic construct
MEEEEYVVEKVLDRRVVKGKVEYLLKWKGFSDEDNTWEPEENLDCPDLIAEFLQSQKTAHETDKSGGGSGGSGSMDAKSLTAWSRTLVTFKDVFVDFTREEWKLLDTAQQIVYRNVMLENYKNLVSLGYQLTKPDVILRLEKGEEPWLVSGGGSGSSGS

SEQ ID NO: 68 - protein, 159 aa, synthetic construct
MEEDEYVVEKVLDRRVVKGKVEYLLKWEGFSDEDNTWEPEENLDCPDLIAEFLQSQKTAHETDKSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 69 - protein, 159 aa, synthetic construct
MGEQVFAAECILSKRLRKGKLEYLVKWKGWSSKHNSWEPEENILDPRLLLAFQKKEHEKEVQNRSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 70 - protein, 159 aa, synthetic construct
MGEDVFAAECILSKRLRKGKLEYLVKWEGWSSKHNSWEPEENILDPRLLLAFQKKEHEKEVQNRSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 71 - protein, 154 aa, synthetic construct
MEEAEPEEFVVEKVLDRRVVNGKVEYFLKWKGFTDADNTWEPEENLDCPELIEAFLNSQKGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 72 - protein, 155 aa, synthetic construct
MEEAEPDEFVVEKVLDRRVVNGKVEYFLKWEGFTDADNTWEPEENLDCPELIEAFLNSQKSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 73 - protein, 225 aa, synthetic construct
MEEAEPDEFVVEKVLDRRVVNGKVEYFLKWEGFTDADNTWEPEENLDCPELIEAFLNSQKSGGGSGGSGSMEEAEPDEFVVEKVLDRRVVNGKVEYFLKWEGFTDADNTWEPEENLDCPELIEAFLNSQKSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 74 - protein, 183 aa, synthetic construct
MEEAEPDEFVVEKVLDRRVVNGKVEYFLKWEGFTDADNTWEPEENLDCPELIEAFLNSQKSRSSGGGSGGSGSMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLSGGGSGGSGS

SEQ ID NO: 75 - protein, 158 aa, synthetic construct
MEEEYVVEKVLDRRVVKGQVEYLLKWKGFSEEHNTWEPEKNLDCPELISEFMKKYKKMKEGENSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 76 - protein, 158 aa, synthetic construct
MEDEYVVEKVLDRRVVKGQVEYLLKWEGFSEEHNTWEPEKNLDCPELISEFMKKYKKMKEGENSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 77 - protein, 231 aa, synthetic construct
MEDEYVVEKVLDRRVVKGQVEYLLKWEGFSEEHNTWEPEKNLDCPELISEFMKKYKKMKEGENSGGGSGGSGSMEDEYVVEKVLDRRVVKGQVEYLLKWEGFSEEHNTWEPEKNLDCPELISEFMKKYKKMKEGENSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 78 - protein, 159 aa, synthetic construct
MVGERFAAESIIKRRIRKGRIEYLVKWKGWAIKYSTWEPEENILDSRLIAAFEQKERERELYGPSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 79 - protein, 160 aa, synthetic construct
MVGDRVFAAESIIKRRIRKGRIEYLVKWEGWAIKYSTWEPEENILDSRLIAAFEQKERERELYGPSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 80 - protein, 168 aa, synthetic construct
MSAIGEQVFAVESIRKKRVRKGKVEYLVKWKGWPPKYSTWEPEEHILDPRLVMAYEEKEERDRASGYRKRGPKSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 81 - protein, 168 aa, synthetic construct
MSAIGEDVFAVESIRKKRVRKGKVEYLVKWEGWPPKYSTWEPEEHILDPRLVMAYEEKEERDRASGYRKRGPKSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 82 - protein, 251 aa, synthetic construct
MSAIGEQVFAVESIRKKRVRKGKVEYLVKWKGWPPKYSTWEPEEHILDPRLVMAYEEKEERDRASGYRKRGPKSGGGSGGSGSMSAIGEQVFAVESIRKKRVRKGKVEYLVKWKGWPPKYSTWEPEEHILDPRLVMAYEEKEERDRASGYRKRGPKSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 83 - protein, 159 aa, synthetic construct
MGERVFAAEALLKRRIRKGRMEYLVKWKGWSQKYSTWEPEENILDARLLAAFEEREREMELYGPSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 84 - protein, 159 aa, synthetic construct
MGEDVFAAEALLKRRIRKGRMEYLVKWEGWSQKYSTWEPEENILDARLLAAFEEREREMELYGPSGGGSGGSGSMDAKSLTAWSRTLVTFKDVFVDFTREEWKLLDTAQQIVYRNVMLENYKNLVSLGYQLTKPDVILRLEKGEEPWLVSGGGSGGSGS

SEQ ID NO: 85 - protein, 10 aa, synthetic construct
SGGGSGGSGS

SEQ ID NO: 86 - DNA, 22 bp, synthetic construct
  source 1..22: mol_type = other DNA
gaaagtccccaggctccccagc

SEQ ID NO: 87 - DNA, 20 bp, synthetic construct
  source 1..20: mol_type = other DNA
gcctggcaggatgcgcggtg

SEQ ID NO: 88 - DNA, 20 bp, synthetic construct
  source 1..20: mol_type = other DNA
gggagccccgtagaaccgag

SEQ ID NO: 89 - DNA, 20 bp, synthetic construct
  source 1..20: mol_type = other DNA
caccgccctcctccccacgg

SEQ ID NO: 90 - DNA, 20 bp, synthetic construct
  source 1..20: mol_type = other DNA
tgtcggtgaagcggcagtgg

SEQ ID NO: 91 - DNA, 20 bp, synthetic construct
  source 1..20: mol_type = other DNA
caggcgggttcaggcttcgg

SEQ ID NO: 92 - protein, 72 aa, synthetic construct
SAIGDDIFAVESIRKKRVRKGKVEYLVKWEGWPLEYSTWEPEEHILDPRLVMAYEEKEERDRASGYRKRGPK

SEQ ID NO: 93 - protein, 72 aa, synthetic construct
SAICDNVYPVESIRKKRVRKGKVEYLVKWKGWPLEYSTWEPEEHILDPRLVMAYEEKEERDRASGYRKRGPK

SEQ ID NO: 94 - protein, 72 aa, synthetic construct
SAICEYIFSVESIRKKRVRKGKVEYLVKWEGWPPEYMTWEPEEHILDPRLVMAYEEKEERDRASGYRKRGPK

SEQ ID NO: 95 - protein, 72 aa, synthetic construct
SAICDNVFAVESIRKKRVRKGKVEYLVKWEGWPQEYSTWEPEEHILDPRLVMAYEEKEERDRASGYRKRGPK

SEQ ID NO: 96 - protein, 72 aa, synthetic construct
SAIGHDVFPVESIRKKRVRKGKVEYLVKWRDWPEQYSTWEPEEHILDPRLVMAYEEKEERDRASGYRKRGPK

SEQ ID NO: 97 - protein, 72 aa, synthetic construct
SAIFEDVFPVESIRKKRVRKGKVEYLVKWKGWPSEYSTWEPEEHILDPRLVMAYEEKEERDRASGYRKRGPK

SEQ ID NO: 98 - protein, 6 aa, synthetic construct
HHHHHH

SEQ ID NO: 99 - protein, 6 aa, Homo sapiens
GEQVFA

SEQ ID NO: 100 - protein, 10 aa, Homo sapiens
WKGWPPKYST

SEQ ID NO: 101 - protein, 6 aa, synthetic construct
GDDIFA

SEQ ID NO: 102 - protein, 10 aa, synthetic construct
WEGWPLEYST

SEQ ID NO: 103 - protein, 6 aa, synthetic construct
CDNVYP

SEQ ID NO: 104 - protein, 10 aa, synthetic construct
WKGWPLEYST

SEQ ID NO: 105 - protein, 6 aa, synthetic construct
CEYIFS

SEQ ID NO: 106 - protein, 10 aa, synthetic construct
WEGWPPEYMT

SEQ ID NO: 107 - protein, 6 aa, synthetic construct
CDNVFA

SEQ ID NO: 108 - protein, 10 aa, synthetic construct
WEGWPQEYST

SEQ ID NO: 109 - protein, 6 aa, synthetic construct
GHDVFP

SEQ ID NO: 110 - protein, 10 aa, synthetic construct
WRDWPEQYST

SEQ ID NO: 111 - protein, 6 aa, synthetic construct
FEDVFP

SEQ ID NO: 112 - protein, 10 aa, synthetic construct
WKGWPSEYST

SEQ ID NO: 113 - protein, 64 aa, Homo sapiens
GEDVFAVESIRKKRVRKGKVEYLVKWEGWPPKYSTWEPEEHILDPRLVMAYEEKEERDRASGYR

SEQ ID NO: 114 - protein, 64 aa, Homo sapiens
GEQVFAVESIRKKRVRKGKVEYLVKWKGWPPKYSTWEPEEHILDPRLVMAYEEKEERDRASGYR

SEQ ID NO: 115 - protein, 63 aa, Homo sapiens
EPEEFVVEKVLDRRVVNGKVEYFLKWKGFTDADNTWEPEENLDCPELIEAFLNSQKAGKEKDG

SEQ ID NO: 116 - protein, 63 aa, Homo sapiens
GEHVFAVESIEKKRIRKGRVEYLVKWRGWSPKYNTWEPEENILDPRLLIAFQNRERQEQLMGY

SEQ ID NO: 117 - protein, 63 aa, Homo sapiens
SEEEYVVEKVLDRRVVKGQVEYLLKWKGFSEEHNTWEPEKNLDCPELISEFMKKYKKMKEGEN

SEQ ID NO: 118 - protein, 63 aa, Homo sapiens
VGERFAAESIIKRRIRKGRIEYLVKWKGWAIKYSTWEPEENILDSRLIAAFEQKERERELYGP

(19) United States
(12) Patent Application Publication
(10) Pub. No.: US 2026/0266833 A1
(43) Pub. Date: Sep. 10, 2026

(54) HIGH-AFFINITY ENGINEERED CHROMODOMAINS

(71) Applicant: Axoiya Inc., Palo Alto, CA (US)

(72) Inventors: Sachdev S. SIDHU, Toronto (CA); Donald Gregory MARTYN, Ontario (CA); Gianluca VEGGIANI, Ontario (CA)

(21) Appl. No.: 19/670,417

(22) Filed: May 7, 2026

Related U.S. Application Data
(63) Continuation of application No. PCT/US2024/054880, filed on Nov. 7, 2024.
(60) Provisional application No. 63/596,813, filed on Nov. 7, 2023.

(51) Int. Cl. G01N 33/68; C07K 14/47

(52) U.S. Cl. CPC G01N 33/68; C07K 14/47; G01N 2440/12

(57) ABSTRACT

The present disclosure provides for engineered chromodomains for enhanced binding affinity to methylated proteins, their use in screening, gene transcription regulation, and in methods of their administration. The compositions and methods provided herein can be used for targeting protein methylation as a means for identifying markers of pathology for diagnostic or therapeutic applications.

### CROSS-REFERENCE

This application is a continuation of International Application No. PCT/US2024/054880, filed Nov. 7, 2024, which claims the benefit of U.S. Provisional Application No. 63/596,813 filed Nov. 7, 2023, which is incorporated by reference herein in their entirety.

### SEQUENCE LISTING

The instant application contains a Sequence Listing which has been submitted electronically in XML format and is hereby incorporated by reference in its entirety. Said XML copy, created on Oct. 29, 2024, is named 219439-702601_PCT_SL.xml and is 117,161 bytes in size.

### BACKGROUND

Post-translational modifications (PTMs) are covalent modifications of peptides and proteins that expand and diversify protein function beyond their coding genome. Protein methylation is one type of PTM in prokaryotes and eukaryotes and plays important roles in gene transcription, DNA replication, DNA repair, and cell cycle control. Methylation of non-histone proteins are associated with regulation of a variety of pathways. Aberrant protein methylation has been described in a large variety of human pathologies but deciphering the functional role of lysine methylation remains a major challenge, due to the lack of robust technologies for probing and analyzing methylated proteins.

Conventional antibodies that recognize methylated lysine (mLys) residues suffer from low affinity, poor specificity, and lot-to-lot variations. Such issues create challenges for the analysis of methylation sites, particularly in live cells. As an alternative, several groups have exploited naturally occurring methyllysine-binding domains (reader domains) as tools for the detection of methyllysine-containing proteins. However, the intrinsically weak affinity and specificity for discrete methylated states (e.g., mono-, di-, or tri-methylation) has limited the application of these natural domains. Thus, there is a need to develop new tools for analyzing methylation sites and regulating gene transcription.

### SUMMARY

Provided herein are methods of detecting protein methylation in a biological sample comprising: (a) providing a biological sample; (b) contacting the biological sample with an engineered chromodomain that comprises a methyllysine-binding pocket comprising at least one amino acid substitution as compared to a reference chromodomain selected from any one of SEQ ID NOS: 21-28, wherein the engineered chromodomain provides for enhanced binding to at least one methylated protein compared to said reference chromodomain; and (c) upon the contacting, detecting interaction of the biological sample with the engineered chromodomain, wherein the interaction of the engineered chromodomain with the biological sample indicates a methylated protein in the biological sample. Provided herein are methods, wherein the at least one amino acid substitution in the methyllysine-binding pocket comprises conversion of one or more neutral or positively charged amino acid residues to one or more negatively charged amino acid residues. Provided herein are methods, wherein the at least one amino acid substitution in the methyllysine-binding pocket comprises conversion of an evolutionary conserved Gln to Asp or Glu. Provided herein are methods, wherein the at least one amino acid substitution in the methyllysine-binding pocket comprises conversion of an evolutionary conserved Gln to Asp. Provided herein are methods, wherein the at least one amino acid substitution in the methyllysine-binding pocket comprises conversion of an evolutionary conserved Lys to Asp or Glu. Provided herein are methods, wherein the at least one amino acid substitution in the methyllysine-binding pocket comprises conversion of an evolutionary conserved Lys to Glu. Provided herein are methods, wherein the at least one amino acid substitution in the methyllysine-binding pocket comprises conversion of an evolutionary conserved Gln to Asp and conversion of an evolutionary conserved Lys to Glu. Provided herein are methods, wherein the engineered chromodomain binds to a methylated protein with a dissociation constant (Kd) of 100 μM or less. Provided herein are methods, wherein the engineered chromodomain binds to a methylated protein with a dissociation constant (Kd) of 50 μM or less. Provided herein are methods, wherein the engineered chromodomain binds to the methylated protein with a dissociation constant (Kd) of 10 μM or less. Provided herein are methods, wherein the engineered chromodomain binds to the methylated protein with a dissociation constant (Kd) of 1 μM or less. Provided herein are methods, wherein the engineered chromodomain is selected from a group consisting of Cbx1, Cbx2, Cbx3, Cbx4, Cbx5, Cbx6, Cbx7 and Cbx8. Provided herein are methods, wherein the methylated protein is a histone protein. Provided herein are methods, wherein the histone protein is a histone H3 protein. Provided herein are methods, wherein the amino acid substitutions provide for an enhanced binding of a methylated histone H3 protein at positions 9 (H3K9) and/or 27 (H3K27). Provided herein are methods, where the methylated H3 protein is monomethylated, dimethylated or trimethylated. Provided herein are methods, wherein the amino acid substitutions provide for enhanced H3K27me3 binding to the engineered chromodomain compared to an otherwise identical chromodomain that lacks the at least one amino acid substitution. Provided herein are methods, wherein the amino acid substitutions provide for enhanced H3K9me3 binding to the engineered chromodomain compared to an otherwise identical chromodomain that lacks the at least one amino acid substitution. Provided herein are methods, wherein the amino acid substitutions provide for enhanced H3K9me3 and H3K27me3 binding to the engineered chromodomain compared to an otherwise identical chromodomain that lacks the at least one amino acid substitution. Provided herein are methods, wherein the methylated protein is a non-histone protein. Provided herein are methods, wherein the engineered chromodomain comprises an amino acid sequence having at least 80%, at least 85%, at least 90%, at least 95%, or at least 99% sequence identity to an amino acid sequence selected from SEQ ID NOS: 29-40. Provided herein are methods, wherein the engineered chromodomain comprises an amino acid sequence selected from SEQ ID NOS: 29-40. Provided herein are methods, wherein the detecting comprises applying an assay with quantifiable or semi-quantifiable spectroscopic, photochemical, biochemical, immunochemical, or chemical means technique. Provided herein are methods, wherein the engineered chromodomain further comprises a detectable label. Provided herein are methods, wherein the assay quantifies a difference between a signal from the detectable label upon the interaction and a signal from the detectable label in a normal control sample, wherein the normal control sample does not comprise methylated proteins. Provided herein are methods, wherein the assay is mass spectrometry (MS), tandem mass spectrometry (MS-MS), MS3 analysis, SPECT, CT and PET imaging, enzyme linked immunosorbent assay (ELISA), or luciferase assay. Provided herein are methods, wherein the detectable label is a radioactive label. Provided herein are methods, wherein the detectable label is a fluorescent label. Provided herein are methods, wherein the fluorescent label is fluorescein, rhodamine, or Texas Red. Provided herein are methods, wherein the fluorescent label is a fluorescent protein. Provided herein are methods, wherein the detectable label is a biotin-based label. Provided herein are methods, wherein the detectable label is an electron-dense reagent. Provided herein are methods, wherein the detectable label is an enzyme. Provided herein are methods, wherein the enzyme is alkaline phosphatase, horseradish peroxidase, or luciferase.

Provided herein are engineered chromodomains that comprise a methyllysine-binding pocket comprising at least one amino acid substitution as compared to a reference chromodomain selected from any one of SEQ ID NOS: 21-28, wherein the engineered chromodomain provides for enhanced binding to at least one methylated protein compared to said reference chromodomain. Further provided herein are chromodomains, wherein the at least one amino acid substitutions in the methyllysine-binding pocket comprises conversion of one or more neutral or positively charged amino acid residues to one or more negatively charged amino acid residues. Further provided herein are chromodomains, wherein the at least one amino acid substitutions in the methyllysine-binding pocket comprises conversion of an evolutionary conserved Gln to Asp or Glu. Further provided herein are chromodomains, wherein the at least one amino acid substitutions in the methyllysine-binding pocket comprises conversion of an evolutionary conserved Gln to Asp. In some embodiments, the at least one amino acid substitutions in the methyllysine-binding pocket comprises conversion of an evolutionary conserved Lys to Asp or Glu. Further provided herein are chromodomains, wherein the at least one amino acid substitutions in the methyllysine-binding pocket comprises conversion of an evolutionary conserved Lys to Glu. Further provided herein are chromodomains, wherein the at least one amino acid substitutions in the methyllysine-binding pocket comprises conversion of an evolutionary conserved Gln to Asp and conversion of an evolutionary conserved Lys to Glu. Further provided herein are chromodomains, wherein the engineered chromodomain binds to a methylated protein with a dissociation constant (Kd) of 100 μM or less. Further provided herein are chromodomains, wherein the engineered chromodomain binds to a methylated protein with a dissociation constant (Kd) of 50 μM or less. Further provided herein are chromodomains, wherein the engineered chromodomain binds to a methylated protein with a dissociation constant (Kd) of 10 μM or less. Further provided herein are chromodomains, wherein the engineered chromodomain binds to a methylated protein with a dissociation constant (Kd) of 1 μM or less. Further provided herein are chromodomains, wherein the engineered chromodomain is selected from a group consisting of Cbx1, Cbx2, Cbx3, Cbx4, Cbx5, Cbx6, Cbx7 and Cbx8. Further provided herein are chromodomains, wherein the methylated protein is a histone protein. In some embodiments, the histone protein is a histone H3 protein Further provided herein are chromodomains, wherein the amino acid substitutions provide for enhanced binding of a methylated histone H3 protein at positions 9 (H3K9) and/or 27 (H3K27). Further provided herein are chromodomains, wherein the methylated H3 protein is monomethylated, dimethylated or trimethylated. Further provided herein are chromodomains, wherein the amino acid substitutions provide for enhanced H3K27me3 binding to the engineered chromodomain compared to an otherwise identical chromodomain that lacks the at least one amino acid substitution. Further provided herein are chromodomains, wherein the amino acid substitutions provide for enhanced H3K9me3 binding to the engineered chromodomain compared to an otherwise identical chromodomain that lacks the at least one amino acid substitution. Further provided herein are chromodomains, wherein the amino acid substitutions provide for enhanced H3K9me3 and H3K27me3 binding to the engineered chromodomain compared to an otherwise identical chromodomain that lacks the at least one amino acid substitution. Further provided herein are chromodomains, wherein the methylated protein is a non-histone protein. Further provided herein are chromodomains, wherein the engineered chromodomain comprises an amino acid sequence having at least 80%, at least 85%, at least 90%, at least 95%, or at least 99% sequence identity to an amino acid sequence selected from SEQ ID NOS: 29-44. Further provided herein are chromodomains, wherein the engineered chromodomain comprises an amino acid sequence selected from SEQ ID NOS: 29-44. Further provided herein are chromodomains, wherein the engineered chromodomains described herein comprise a detectable label. Further provided herein are chromodomains, wherein the detectable label is a radioactive label. Further provided herein are chromodomains, wherein the detectable label is a fluorescent label. Further provided herein are chromodomains, wherein the fluorescent label is fluorescein, rhodamine, or Texas Red. Further provided herein are chromodomains, wherein the fluorescent label is a fluorescent protein. In some embodiments, the detectable label is a biotin-based label. Further provided herein are chromodomains, wherein the detectable label is an electron-dense reagent. In some embodiments, the detectable label is an enzyme. Further provided herein are chromodomains, wherein the enzyme is alkaline phosphatase, horseradish peroxidase, or luciferase. Further provided herein are chromodomains, wherein the engineered chromodomain described herein further comprises comprising a nuclear localization signal (NLS).

Provided herein are libraries comprising the engineered chromodomains as described herein and a plurality of methylated proteins.

Provided herein are nucleic acids, wherein the nucleic acids encode for the engineered chromodomains as described herein.

Provided herein are methods for isolating a methylated lysine containing polypeptide from a complex mixture of peptides, the method comprising: (a) contacting a proteinaceous preparation with an engineered chromodomain that comprises a methyllysine-binding pocket comprising at least one amino acid substitution as compared to a reference chromodomain selected from any one of SEQ ID NOS: 21-28, wherein the engineered chromodomain provides for enhanced binding to at least one methylated protein compared to said reference chromodomain, and (b) isolating methylated lysine containing polypeptide bound to any of the engineered chromodomains of step (a). Further provided herein are methods, wherein the at least one amino acid substitutions in the methyllysine-binding pocket comprises conversion of one or more neutral or positively charged amino acid residues to one or more negatively charged amino acid residues. Further provided herein are methods, wherein the at least one amino acid substitutions in the methyllysine-binding pocket comprises conversion of an evolutionary conserved Gln to Asp or Glu. Further provided herein are methods, wherein the at least one amino acid substitutions in the methyllysine-binding pocket comprises conversion of an evolutionary conserved Gln to Asp. Further provided herein are methods, the at least one amino acid substitutions in the methyllysine-binding pocket comprises conversion of an evolutionary conserved Lys to Asp or Glu. Further provided herein are methods, wherein the at least one amino acid substitutions in the methyllysine-binding pocket comprises conversion of an evolutionary conserved Lys to Glu. Further provided herein are methods, wherein the at least one amino acid substitutions in the methyllysine-binding pocket comprises conversion of an evolutionary conserved Gln to Asp and conversion of an evolutionary conserved Lys to Glu. Further provided herein are methods, wherein the engineered chromodomain binds to a methylated protein with a dissociation constant (Kd) of 100 μM or less. Further provided herein are methods, wherein the engineered chromodomain binds to a methylated protein with a dissociation constant (Kd) of 50 μM or less. Further provided herein are methods, wherein the engineered chromodomain binds to a methylated protein with a dissociation constant (Kd) of 10 μM or less. Further provided herein are methods, wherein the engineered chromodomain binds to a methylated protein with a dissociation constant (Kd) of 1 μM or less. Further provided herein are methods, wherein the engineered chromodomain is selected from a group consisting of Cbx1, Cbx2, Cbx3, Cbx4, Cbx5, Cbx6, Cbx7 and Cbx8. Further provided herein are methods, wherein the methylated protein is a histone protein. In some embodiments, the histone protein is a histone H3 protein. Further provided herein are methods, wherein the amino acid substitutions provide for enhanced binding of a methylated histone H3 protein at positions 9 (H3K9) and/or 27 (H3K27). Further provided herein are methods, wherein the methylated H3 protein is monomethylated, dimethylated or trimethylated. Further provided herein are methods, wherein the amino acid substitutions provide for enhanced H3K27me3 binding to the engineered chromodomain compared to an otherwise identical chromodomain that lacks the at least one amino acid substitution. Further provided herein are methods, wherein the amino acid substitutions provide for enhanced H3K9me3 binding to the engineered chromodomain compared to an otherwise identical chromodomain that lacks the at least one amino acid substitution. Further provided herein are methods, wherein the amino acid substitutions provide for enhanced H3K9me3 and H3K27me3 binding to the engineered chromodomain compared to an otherwise identical chromodomain that lacks the at least one amino acid substitution. Further provided herein are methods, wherein the methylated protein is a non-histone protein. Further provided herein are methods, wherein the engineered chromodomain comprises an amino acid sequence having at least 80%, at least 85%, at least 90%, at least 95%, or at least 99% sequence identity to an amino acid sequence selected from SEQ ID NOS: 29-44. Further provided herein are methods, wherein the engineered chromodomain comprises an amino acid sequence selected from SEQ ID NOS: 29-44. Further provided herein are methods, wherein the engineered chromodomain is immobilized to streptavidin beads. Further provided herein are methods, wherein the engineered chromodomain is biotinylated. Further provided herein are methods, wherein the engineered chromodomain is fused to a tag. Further provided herein are c methods, wherein the tag is a hexa-histidine tag. Further provided herein are methods, wherein the tag is a GST-tag. Further provided herein are methods, wherein the proteinaceous preparation is from organisms. Further provided herein are methods, wherein the organisms are single cells, multicellular organisms, tissues, organs, or bacteria Further provided herein are methods, wherein the proteinaceous preparation is from bodily fluids. Further provided herein are methods, wherein the bodily fluids are blood, tears, spinal fluid, synovial fluid, bronchoalveolar fluid, bronchoalveolar lavage, tissue extracts, urine, sweat, saliva, excrement, or phlegm.

Provided herein are systems for repressing gene transcription, wherein the system comprises: (a) an engineered chromodomain that comprises a methyllysine-binding pocket comprising at least one amino acid substitution as compared to a reference chromodomain selected from any one of SEQ ID NOS: 21-28, wherein the engineered chromodomain provides for enhanced binding to at least one methylated protein compared to said reference chromodomain; (b) a transcriptional regulator; (c) a DNA targeting domain; and (d) a guide nucleic acid, wherein the guide nucleic acid comprises a targeting sequence complementary to a garget nucleic acid sequence of a gene, and wherein the engineered chromodomain enhances the repression of the gene by the transcriptional regulator. Further provided herein are systems, wherein the at least one amino acid substitutions in the methyllysine-binding pocket comprises conversion of one or more neutral or positively charged amino acid residues to one or more negatively charged amino acid residues. Further provided herein are systems, wherein the at least one amino acid substitutions in the methyllysine-binding pocket comprises conversion of an evolutionary conserved Gln to Asp or Glu. Further provided herein are systems, wherein the at least one amino acid substitutions in the methyllysine-binding pocket comprises conversion of an evolutionary conserved Gln to Asp. Further provided herein are systems, the at least one amino acid substitutions in the methyllysine-binding pocket comprises conversion of an evolutionary conserved Lys to Asp or Glu. Further provided herein are systems, wherein the at least one amino acid substitutions in the methyllysine-binding pocket comprises conversion of an evolutionary conserved Lys to Glu. Further provided herein are systems, wherein the at least one amino acid substitutions in the methyllysine-binding pocket comprises conversion of an evolutionary conserved Gln to Asp and conversion of an evolutionary conserved Lys to Glu. Further provided herein are systems, wherein the engineered chromodomain binds to a methylated protein with a dissociation constant (Kd) of 100 μM or less. Further provided herein are systems, wherein the engineered chromodomain binds to a methylated protein with a dissociation constant (Kd) of 50 μM or less. Further provided herein are systems, wherein the engineered chromodomain binds to a methylated protein with a dissociation constant (Kd) of 10 μM or less. Further provided herein are systems, wherein the engineered chromodomain binds to a methylated protein with a dissociation constant (Kd) of 1 μM or less. Further provided herein are systems, wherein the engineered chromodomain is selected from a group consisting of Cbx1, Cbx2, Cbx3, Cbx4, Cbx5, Cbx6, Cbx7 and Cbx8. Further provided herein are systems, wherein the methylated protein is a histone protein. In some embodiments, the histone protein is a histone H3 protein. Further provided herein are methods, wherein the amino acid substitutions provide for enhanced binding of a methylated histone H3 protein at positions 9 (H3K9) and/or 27 (H3K27). Further provided herein are methods, wherein the methylated H3 protein is monomethylated, dimethylated or trimethylated. Further provided herein are methods, wherein the amino acid substitutions provide for enhanced H3K27me3 binding to the engineered chromodomain compared to an otherwise identical chromodomain that lacks the at least one amino acid substitution. Further provided herein are methods, wherein the amino acid substitutions provide for enhanced H3K9me3 binding to the engineered chromodomain compared to an otherwise identical chromodomain that lacks the at least one amino acid substitution. Further provided herein are methods, wherein the amino acid substitutions provide for enhanced H3K9me3 and H3K27me3 binding to the engineered chromodomain compared to an otherwise identical chromodomain that lacks the at least one amino acid substitution. Further provided herein are methods, wherein the methylated protein is a non-histone protein. Further provided herein are methods, wherein the engineered chromodomain comprises an amino acid sequence having at least 80%, at least 85%, at least 90%, at least 95%, or at least 99% sequence identity to an amino acid sequence selected from SEQ ID NOS: 29-44. Further provided herein are methods, wherein the engineered chromodomain comprises an amino acid sequence selected from SEQ ID NOS: 29-44. Further provided herein are chromodomains, wherein the DNA targeting domain is an enzymatically inactive Cas9 (dCas9), enzymatically inactive 12a (dCpf1), a zinc finger domain, or a TAL effector molecule. Further provided herein are chromodomains, wherein the DNA targeting domain is an enzymatically inactive Cas9 (dCas9). Further provided herein are chromodomains, wherein the transcriptional regulator comprises a Krüppel associated box (KRAB) repressor domain. Further provided herein are systems, wherein the transcriptional repressor is KOX1, ZIM3, ZIM2, ZNF554, ZNF264, ZNF324, ZNF354, ZFP82, or ZNF669. Further provided herein are systems, wherein the transcriptional repressor is Zinc finger protein 1 (KOX1). Further provided herein are systems, wherein the transcriptional repressor is Zinc Finger Imprinted 3 (ZIM3). Further provided herein are systems, wherein the KRAB repressor domain comprises a sequence at least 80%, at least 85%, at least 90%, or at least 95% sequence identity to an amino acid sequence of SEQ ID NO: 53. Further provided herein are systems, wherein the KRAB repressor domain comprises an amino acid sequence of SEQ ID NO: 54. Further provided herein are systems, wherein the engineered chromodomain is linked to the transcriptional regulator via a peptide linker. Further provided herein are systems, wherein the expression of the gene is reduced by at least 50%, at least 55%, at least 60%, at least 65%, at least 70%, at least 75%, at least 80%, at least 85%, or about 90%.

Provided herein are polynucleotides or sets of polynucleotides encoding the systems as described herein.

Provided herein are vectors comprising the polynucleotides or the sets of polynucleotides as described herein. Further provided herein are chromodomains, wherein the vector is a lentiviral vector, an adenoviral vector, an adeno-associated viral vector (AAV), a recombinant AAV (rAAV), a retroviral vector, or a minicircle vector.

Provided herein are cells comprising the systems, the polynucleotides or sets of polynucleotides, or the vectors as described herein.

Provided herein are composition comprising the engineered chromodomains described herein for use of detecting a presence of a methylated protein in a biological sample.

Provided herein are kits comprising the engineered domains, the libraries, the nucleic acids, or the systems as described herein.

Provided herein are methods of repressing gene transcription, wherein the methods comprise contacting cells with the systems, the polynucleotides, the sets of polynucleotides, the vectors as described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D are plots, graphs and charts showing the design and characterization of optimized Cbx7 chromodomains. FIG. 1A is a plot showing phage ELISA binding curves of different chromodomains to an immobilized anti-Flag tag antibody; the x-axis represents the phage concentration; the y-axis represents Absorbance at 450 nm (mean of triplicate 1+SD). FIG. 1B is a graph showing the design of the phage-displayed library mapped onto the structure of Cbx7-chromo in complex with a H3K27me3 peptide (PDB code: 4X3K). FIG. 1C is a bar plot showing ELISAs for phage-displayed Cbx7. Vs binding to immobilized H3K27me3 peptide, H3K27 peptide or streptavidin (x-axis) and the y-axis represents Absorbance at 450 nm. Data represent the mean and standard deviation of triplicate measurements. FIG. 1D is a heatmap showing the phage ELISAs for assessment of specificity of Flag-tagged Cbx7.

FIGS. 2A-2C are graphs illustrating specificities of wild-type and high-affinity chromodomains of PcG and HP1 family members. FIG. 2A is a graph showing the sequence alignment of all chromodomains of Cbx proteins and Cbx7.VD. Numbering was based on full length Cbx7.wt, conserved residues are shaded in dark, whereas amino acids forming the aromatic cage are shaded in light grey. Positions that were mutated as in Cbx7. VD are shaded are indicated by triangles above the sequence. Figure discloses SEQ ID NOs 113-114, 21-22, 115-118, and 28, respectively, in order of appearance. FIG. 2B is a graph showing the superposition of all human Cbx chromodomain structures with that of Cbx7 in complex with an H3K27me3 peptide (PDB: 4X3K). Mainchains of all domains are represented as gray ribbons, and sidechains of residues at positions 9 and 33 are shown as sticks (Cbx1, PDB: 3F2U; Cbx2, PDB: 3H91; Cbx3, PDB: 3DM1; Cbx4, PDB: 5EPL; Cbx5, PDB: 3FDT; Cbx6, PDB: 3190; Cbx7, PDB: 4X3K; Cbx8, PDB: 3191). Side chains at position 9 of Cbx1, Cbx3, and Cbx6 are missing, as they were not visible in the structures. FIG. 2C is a graph showing the specificity profiling of Cbx chromodomain-luciferase fusions with a panel of methylated and non-methylated histone peptides. Luminescence intensity for each domain was normalized to intensity for the H3K9me3 peptide, except for Cbx2.wt and Cbx2.VD, for which it was normalized to intensity for the H3K27me3 peptide. The mean of normalized luminescence intensity from three independent experiments is shown in a gradient.

FIGS. 3A-3B are an image and a plot showing the thermostability of Cbx4.wt and Cbx4.BD. FIG. 3A is a representative SDS-PAGE gel showing decreased thermostability of Cbx4.VD. FIG. 3B is a histogram bar plot showing the % of soluble chromodomain (Y-axis) determined by densitometry at different temperatures (X-axis). Data were analyzed from three independent experiments and expressed as mean±SD.

FIGS. 4A-4E are graphs and plots illustrating the functional characterization of engineered chromodomains in mouse embryonic stem cells (mESCs). FIG. 4A is a schematic representation of RCME strategy for site-specific integration of monovalent and bivalent Cbx chromodomain variants in the genome of mESCs. Site-specific biotinylation of stably integrated chromodomains was performed in vivo by the enzymatic activity of an E. coli biotin ligase (BirA). FIG. 4B is live-cell imaging of mESCs expressing Cbx7.VD and Cbx2.VD chromodomains arranged in single domain or double-domain configuration. Nuclear EGFP was used as a control. Data show representative confocal images from two independent experiments. Scale bar is 5 μm. FIG. 4C is a genome browser plot showing correct localization of engineered chromodomains at a H3K27me3 genomic region detected by antibody chromatin immunoprecipitation (ChIP) assays with sequencing (ChIP-seq). Shown is the bigWig coverage-track at 100-bp intervals and normalized to Reads Per Kilobase per Million mapped reads (RPKM). Gene models are indicated. FIG. 4D is a graph depicting average density profiles. In the top panel shows average density profiles of EGFP-fused chromodomains centered at H3K27me3 peaks. Chromodomain variants fused to EGFP were stably integrated into mESCs or Eed-KO cells lacking H3K27me3. Data indicate increased genome-wide binding for high-affinity chromodomains, with Cbx2. VD-2x and Cbx7.VD-2x showing the greatest enrichment. The bottom panel shows the average density plots for EGFP-fused Cbx variants centered at H3K9me3 peaks. FIG. 4E is a Person correlation analysis chart showing genome-wide interaction of chromodomain variants with histone modifications. Pearson correlation was obtained from comparisons of engineered chromodomains to histone modifications at filtered 1-kb-sized genomic bins (n=61,145) on chromosome 19.

FIGS. 5A-5G are graphs and plots showing expression and live-cell imaging of engineered chromodomains in mESCs. FIG. 5A are schematics of the constructs used to obtain mESCs stably expressing engineered chromodomains. Chromodomain variants (Cbx. VDs) are composed of single or double chromodomains fused in frame to EGFP for live-cell imaging experiments. At the N-terminus constructs were fused to a biotin acceptor site for site-specific biotinylation and biotin ChIP-seq experiments. FIG. 5B are flow cytometry profiles of Cbx.wt and Cbx. VDs fused to EGFP stably integrated into mESCs. FIG. 5C shows the live-cell imaging of mESCs stably expressing Cbx3.wt, Cbx3.VD, Cbx5.wt and Cbx5.VD chromodomains fused to EGFP. Data show representative confocal images from two independent experiments. Scale bar 5 μm. FIG. 5D are graphs showing the genome browser examples for loss of binding of engineered chromodomains to H3K27me3 sites. FIG. 5E is a correlation analysis chart showing genome-wide interaction of Cbx. VDs with histone modifications. Pearson correlation score was calculated based on counts at 1 kb intervals. FIG. 5F is live-cell imaging showing nuclear localization of Cbx2. VD-2x and Cbx7.VD-2x in mESCs and Eed-KO cells. FIG. 5G are western blot images showing comparison of the expression levels of Cbx2. VD-2x and Cbx7.VD-2x in mESCs and Eed-KO cells. The expression of each chromodomain was assessed using an anti-EGFP antibody, whereas equal loading was detected with an anti-Lamin B1 antibody. Similar results were obtained from two independent experiments.

FIGS. 6A-6F are graph and plots demonstrating the effects of high-affinity chromodomains on Clustered Regularly Interspaced Short Palindromic Repeats (CRISPR) interference (CRISPRi) efficiency. FIG. 6A is a schematic representation of the reporter cell line and CRISPR-based repressors used in the assay. EGFP expression is driven by an SV40 promoter and Cbx-KRAB-dCas9 fusions are recruited to the SV40-EGFP reporter by stably expressing a gRNA targeting the SV40 promoter. KOX1-dCas9 and Cbx-KOX1-dCas9 repressors were delivered into the reporter cell line by lentiviral infection and EGFP silencing was measured by flow cytometry. FIG. 6B is a bar plot showing EGFP expression levels were measured by flow cytometry 20 days after cell infection. Each bar represents the geometric mean of fluorescence signals relative to untransduced reporter cells (n=6, +1 SD), with each data point showing a biological replicate. FIG. 6C is a plot showing Log2 fold change repression efficiency of Cbx-KOX1-dCas9 fusions in comparison to KOX1-dCas9 (no Cbx). The Y axis corresponds to different fusions or KOX1-dCas9 (no Cbx), whereas the X axis represents the values of Log2 fold changes over KOX1-dCas9. (n=6, p<0.005, two-tailed unpaired t-test). Some error bars are too small to be visible. FIG. 6D is a bar plot comparing the repression by KOX1-dCas9 fused to single (Cbx3.VD, Cbx5.VD, Cbx7.VD) or double (Cbx3.VD-2x, Cbx5.VD-2x, Cbx7.VD-2x) chromodomains. FIG. 6E is an image showing the western blot analysis on the expression levels of different KRAB-dCas9 fusion proteins. Expression of repressors was assayed using an anti-Cas9 antibody. Equal loading was detected through an anti-GAPDH antibody. Data show a representative western blot from three independent experiments. FIG. 6F is a plot showing the time course-dependent EGFP fluorescence intensity changes of EGFP reporter cells after transfection with plasmids encoding dCas9, KOX1-dCas9 or Cbx3. VD-KOX1-dCas9. Y axis indicates Log2 normalized EGFP fluorescence, whereas X axis corresponds to time (h).

FIGS. 7A-7E are plots demonstrating Cbx3.VD enables modular and potent gene repression. FIG. 7A is a bar plot showing relative EGFP levels of HEK293T-SV40 EGFP reporter cells stably transduced with different KRAB domain-containing repressors (KOX1-dCas9, ZIM3-dCas9, and Cbx3. VD-ZIM3-dCas9) and a repressor lacking KRAB domains, obtained by directly fusing Cbx3.VD to dCas9 (Cbx3.VD-dCas9). Histograms show the mean from three independent experiments #1 SD of EGFP levels relative to untransduced reporter cells. FIG. 7B are plots showing repression of endogenous CD81. Left panel shows CD81 surface levels relative to HEK293T cells transduced only with gRNA targeting CD81 promoter (guide only). Data are mean from three independent measurements+1 SD, asterisks indicate p values (two-tailed unpaired t-test, ** p=0.002, *** p=0.0001). Some error bars are too small to be visible. Right panel shows representative flow cytometry profiles of CD81 expression detected with an anti-CD81 PE-conjugated antibody. FIGS. 7C-7E are bar plots showing comparison of the repressive potency of Cbx3.VD-ZIM3-dCas9 with that of ZIM3-dCas9 and dCas9 in HEK293T (FIG. 7C), U20S (FIG. 7D) and HeLa (FIG. 7E) cells. Y axis in each plot represents % relative expression, whereas X axis represents untransduced cells or cells transduced with different repressors.

FIGS. 8A-8C are images showing western blots analysis of the expression levels of ERK1 and ARPC2 proteins upon CRISPRi in HEK293T (FIG. 8A), U20S (FIG. 8B) and HeLa (FIG. 8C) cells. Equal loading was determined using an anti-GAPDH antibody. Similar results were obtained from two independent experiments.

### DETAILED DESCRIPTION OF THE INVENTION

Provided herein are compositions, systems, kits, methods, and uses thereof. Briefly, further described herein are: (1) engineered chromodomains; (2) reagents; (3) probes; (4) vectors and delivery systems; (5) gene repression systems; (6) kits; and (7) other applications.

Provided herein are engineered high affinity chromodomains that can be used in a variety of applications, such as biological process analysis, diagnostics, and therapeutics. The high affinity chromodomains provided herein are engineered to introduce amino acid substitutions for enhanced binding to methylated proteins. Therefore, such engineered high-affinity chromodomains can be used as affinity reagents for methylated histones as well as powerful probes for genome-wide binding analysis and live-cell imaging. Furthermore, the engineered chromodomains provided herein are useful for development of CRISPR-based gene repressors for tailored gene silencing. Such high affinity binding reagents allow for obtaining data/information generation from a highly enriched population correlated to the target-binding ability.

### Definitions

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (in some embodiments “containing,” “including,” “having” and “comprising” typically indicate “including without limitation”). Examples of limiting terms include “consisting of” and “consisting essentially of.” Singular forms including in the claims such as “a,” “an” and “the” include the plural reference unless expressly stated otherwise.

Unless specifically stated or obvious from context, as used herein, the term “about” in reference to a number or range of numbers is understood to include small fluctuations, referring to the stated number and numbers+/−10% thereof.

Unless specifically stated or obvious from context, as used herein, the term “substantially match” is understood to describes a situation that the resulting analysis and comparison is performed to identify resources that are the same; however, in practice the match can correspond to a set of resources that sufficiently similar for the methods describe herein.

The following standard one letter and three letter abbreviations for the amino acid residues may be used throughout the specification: A, Ala—alanine; R, Arg—Arginine; N, Asn—Asparagine; D, Asp—Aspartic acid; C, Cys—Cysteine; Q, Gln—Glutamine; E, Glu—Glutamic acid; G, Gly—Glycine; H, His—Histidine; I, Ile—Isoleucine; L, Leu—Leucine; K, Lys—Lysine; M, Met—Methionine; F, Phe—Phenyalanine; P, Pro—Proline; S, Ser—Serine; T, Thr—Threonine; W, Trp—Tryptophan; Y, Tyr—Tyrosine; and V, Val—Valine.

The term “isolated peptide” or “isolated DNA” refers to a peptide or DNA molecule that has been produced and removed from the source that produced the peptide or DNA, such as recombinant cells or synthesis reactants. The term includes, without limitation, recombinant or cloned DNA isolates and chemically synthesized analogs or analogs biologically synthesized by heterologous systems.

The term “peptide” or “polypeptide” or “oligopeptide” as used herein is defined as a chain of amino acid residues, usually having a defined sequence. As used herein the term “peptide” is mutually inclusive of the terms “polypeptides”, “peptides” and “proteins”.

The term “mutation,” as used herein, refers to a deletion, an insertion of a heterologous nucleic acid, an inversion, or a substitution, including an open reading frame ablating mutations as commonly understood in the art.

The term “gene,” as used herein, refers to a segment of nucleic acid that encodes for an individual protein or RNA (also referred to as a “coding sequence” or “coding region”), optionally together with associated regulatory regions such as promoters, operators, terminators, and the like, which may be located upstream or downstream of the coding sequence.

### (1) Engineered Chromodomains

Provided here are engineered chromodomains comprising at least one amino acid substitution in a methyllysine-binding pocket. In some embodiments, the engineered chromodomain comprises a methyllysine-binding pocket comprising at least one amino acid substitution as compared to a reference chromodomain selected from any one of SEQ ID NOS: 21-28. In some embodiments, the one or more amino acid substitutions provide for enhanced methylated protein binding to the engineered chromodomains. In some embodiments, the engineered chromodomain comprises conversion of one or more neutral or positively charged amino acid residues to one or more negatively charged amino acid residues.

In some embodiments, the engineered chromodomains further comprise at least one amino acid substitutions in the methyllysine-binding pocket comprises conversion of an evolutionary conserved Gln to Asp or Glu. In some embodiments, the at least one amino acid substitutions in the methyllysine-binding pocket comprises conversion of an evolutionary conserved Gln to Asp. In some embodiments, the at least one amino acid substitutions in the methyllysine-binding pocket comprises conversion of an evolutionary conserved Lys to Asp or Glu. In some embodiments, the at least one amino acid substitutions in the methyllysine-binding pocket comprises conversion of an evolutionary conserved Lys to Glu. In some embodiments, the at least one amino acid substitutions in the methyllysine-binding pocket comprises conversion of an evolutionary conserved Gln to Asp and conversion of an evolutionary conserved Lys to Glu.

Protein methylation is one of the most abundant PTMs in prokaryotes and eukaryotes. In cells, lysine methylation and arginine methylation can occur in both histone proteins and non-histone proteins. In some embodiments, the methylated protein described herein is a non-histone protein. In some embodiments, the methylated protein described herein is a methylated histone. In some embodiments, the histone protein is a histone H3 protein. In some embodiments, the methylated H3 protein is monomethylated, dimethylated or trimethylated. In some embodiments, the amino acid substitutions provide for enhanced binding of a methylated histone H3 protein at positions 9 (H3K9) and/or 27 (H3K27). In some embodiments, the amino acid substitutions provide for enhanced H3K27me3 binding to the engineered chromodomain compared to an otherwise identical chromodomain that lacks the at least one amino acid substitution. In some embodiments, the amino acid substitutions provide for enhanced H3K9me3 binding to the engineered chromodomain compared to an otherwise identical chromodomain that lacks the at least one amino acid substitution. In some embodiments, the amino acid substitutions provide for enhanced H3K9me3 and H3K27me3 binding to the engineered chromodomain compared to an otherwise identical chromodomain that lacks the at least one amino acid substitution. In some embodiments, the methylated protein is a methylated non-histone protein.

In some embodiments, the engineered chromodomain binds to a methylated protein with a dissociation constant (Kd) of 100 μM or less. In some embodiments, the engineered chromodomain binds to a methylated protein with a dissociation constant (Kd) of 90 μM or less. In some embodiments, the engineered chromodomain binds to a methylated protein with a dissociation constant (Kd) of 80 μM or less. In some embodiments, the engineered chromodomain binds to a methylated protein with a dissociation constant (Kd) of 70 μM or less. In some embodiments, the engineered chromodomain binds to a methylated protein with a dissociation constant (Kd) of 60 μM or less. In some embodiments, the engineered chromodomain binds to a methylated protein with a dissociation constant (Kd) of 50 μM or less. In some embodiments, the engineered chromodomain binds to a methylated protein with a dissociation constant (Kd) of 40 μM or less. In some embodiments, the engineered chromodomain binds to a methylated protein with a dissociation constant (Kd) of 20 μM or less. In some embodiments, the engineered chromodomain binds to a methylated protein with a dissociation constant (Kd) of 10 μM or less. In some embodiments, the engineered chromodomain binds to a methylated protein with a dissociation constant (Kd) of 1 μM or less.

In some embodiments, the engineered chromodomain is made based on chromodomains selected from a group consisting of from Cbx1, Cbx2, Cbx3, Cbx4, Cbx5, Cbx6, Cbx7 and Cbx8. In some embodiments, the engineered chromodomain comprises an amino acid sequence having at least 80%, at least 85%, at least 90%, at least 95%, or at least 99% sequence identity to an amino acid sequence selected from SEQ ID NOS: 29-40. In some embodiments, the engineered chromodomain comprises an amino acid sequence selected from SEQ ID NOS: 29-40.

In some embodiments, the engineered chromodomains as described herein provide for enhanced binding to methylated proteins compared to an otherwise identical chromodomain that lacks the at least one amino acid substitution. In some embodiments, the methylated protein is a chromatin protein. In some embodiments, the chromatin protein is a histone. In some embodiments the histone protein is histone H3. In some embodiments, the engineered chromodomains as described herein provide for enhanced binding to Histone H3 at positions 4, 9, and 27. In some embodiments, the engineered chromodomains as described herein provide for enhanced binding to Histone H3 at positions 9 (H3K9) and 27 (H3K27). In some embodiments, the engineered chromodomains as described herein provide for enhanced binding to a methylated H3K9 compared to an otherwise identical chromodomain that lacks the at least one amino acid substitution. In some embodiments, the methylated H3K9 is a mono-methylated, a di-methylated or a tri-methylated. In some embodiments, the engineered chromodomains as described herein provide for enhanced binding to a methylated H3K27 compared to an otherwise identical chromodomain that lacks the at least one amino acid substitution. In some embodiments, the methylated H3K27 is a mono-methylated, a di-methylated or a tri-methylated. In some embodiments, the methylated protein a non-chromatin protein. In some embodiments, the methylated protein is a non-histone protein.

### (2) Reagents

Provided here is a protein methylation isolating reagent comprising one or more polypeptides comprising the engineered chromodomain described herein. In some embodiments, the engineered chromodomains are immobilized peptides. In some embodiments, the one or more immobilized peptides are selected from SEQ IDs: 29-40. In some embodiments, the one or more immobilized peptides is immobilized to streptavidin beads. In some embodiments, the immobilized peptides are biotinylated.

In some embodiments, the engineered chromodomain as disclosed herein is used alone (i.e., homogenous mixture) or as a combination (i.e., a heterogenous mixture of various engineered chromodomains, fused to one another using tags (e.g., Spy26/Snoop27 Tags or other similar technologies), or expressed as protein polymers in any combination) to make a universal methylation affinity-purification tool. In some embodiments, the universal methylation affinity-purification tool covers a greater percentage of methylated proteins than conventional methods. A panel of X engineered chromodomains (Tables 3-4; SEQ ID Nos: 29-56) generated using phage display can be used alone or in combination with one another to enrich methylated lysine (mLys) containing peptides or proteins from samples of interest to analyze the methylation marks on peptide and/or proteins from these samples. Any engineered chromodomain produced using the method described above can also be used in the same manner to enrich or probe for mLys-containing peptides and/or proteins.

Also disclosed here are exemplary methods for isolating a mLys-containing polypeptide from a complex mixture of peptides, the method comprising: (a) obtaining a proteinaceous preparation from an organism or bodily fluid; (b) contacting a proteinaceous preparation with any one of the engineered chromodomains described herein, wherein the engineered chromodomain is immobilized; and (c) isolating mLys-containing polypeptides bound by the one or more immobilized engineered chromodomain in step (b). In some embodiments, the organisms are single cells, multicellular organisms (including subjects as this term is defined in this document), tissues, organs, or bacteria. In some embodiments, the bodily fluids are blood (whole blood, blood plasma, blood serum, capillary blood, venous blood), tears, spinal fluid, synovial fluid, bronchoalveolar fluid, bronchoalveolar lavage, tissue extracts, urine, sweat, saliva, excrement, phlegm or other like bodily fluids excreted from organisms. In some embodiments, the methods further comprise (d) characterizing the polypeptides isolated in step (c) by mass spectrometry (MS), tandem mass spectrometry (MS-MS), and/or MS3 analysis. In some embodiments, the method further comprises (e) utilizing a search program to substantially match the spectra obtained for the polypeptides isolated in step (c) during the characterization of step (d) with the spectra for reference peptide sequences, thereby identifying parent proteins of the isolated polypeptide.

Also disclosed here are example methods for detecting protein methylation in a biological sample, the method comprising: (a) providing a biological sample; (b) contacting the biological sample with an engineered chromodomain described herein; and (c) upon the contacting, detecting interaction of the biological sample with the engineered chromodomain, wherein the interaction of the engineered chromodomain with the biological sample indicates a methylated protein in the biological sample. In some embodiments, the interaction indicates changes in intrinsic properties of the biological sample. Intrinsic properties of a biological sample include, but are not limited to, optical properties, cell shapes, cell motility, and morphological traits of cells. In some embodiments, the detecting comprises applying an assay with quantifiable or semi-quantifiable spectroscopic, photochemical, biochemical, immunochemical, or chemical means technique. In some embodiments, the engineered chromodomain further comprises a detectable label. In some embodiments, the assay quantifies a difference between a signal from the detectable label upon the interaction and a signal from the detectable label in a normal control sample, wherein the normal control sample does not comprise methylated proteins. In some embodiments, the assay is mass spectrometry (MS), tandem mass spectrometry (MS-MS), MS3 analysis, SPECT, CT and PET imaging, enzyme linked immunosorbent assay (ELISA), or luciferase assay. In some embodiments, the labels that are detectable includes, without limitation, any material having a detectable physical or chemical property and have been well-developed in the field of immunoassays. In some embodiments, the label is any composition detectable by spectroscopic, photochemical, biochemical, immunochemical, or chemical means.

### (3) Probes

Provided herein are methylation probes comprising at least a peptide comprising an engineered chromodomain and an imaging probe. In some embodiments, this probe is labelled with a detectable marker which may allow detection of the location of mLys-positive cells. In some embodiments, the probe may allow following movement and development of mLys-positive cells. In some embodiments, the imaging component of the probe comprises a label. In some embodiments, the label is suitable for use in in vivo imaging. In some embodiments, the methylation probes are labelled prior to detection. In some embodiments, the label binds to the hybridization product. In some embodiments, the labels are detectable. In some embodiments, the labels that are detectable includes, without limitation, any material having a detectable physical or chemical property and have been well-developed in the field of immunoassays. In some embodiments, the label is any composition detectable by spectroscopic, photochemical, biochemical, immunochemical, or chemical means.

In some embodiments, high affinity chromodomain binding reagents described herein comprise labels. Non-limiting examples of labels include biotin-based label, magnetic label (e.g. DYNABEADS™, MAGNE™ Streptavidin Beads), paramagnetic labels, radioactive label (e.g. ³H, ³⁵S, ³²p, ⁵¹Cr, or ¹²⁵I), fluorescent label (e.g. fluorescein, rhodamine, Texas Red, etc.), fluorescent proteins (i.e. GFP, RFP, CFP) electron-dense reagents (e.g. gold), enzymes (e.g. alkaline phosphatase, horseradish peroxidase, luciferase or others commonly used in an ELISA), digoxigenin, or haptens and proteins for which antisera or monoclonal antibodies may be available (In some embodiments, the peptides of the present disclosure can be made detectable by, In some embodiments, incorporating a radiolabel into the peptide, and used to detect antibodies specifically reactive with the peptide). In some embodiments, the engineered chromodomain as described herein is provided with a carrier. In some embodiments, the engineered chromodomain is coupled to bovine serum albumin (BSA) or keyhole limpet hemocyanin. In some embodiments, the engineered chromodomain is covalently or non-covalently coupled to a solid carrier. In some embodiments, the engineered chromodomain is coupled to a microsphere of gold or polystyrene, a slide, a chip, or to a wall of a microtiter plate. In some embodiments, the engineered chromodomain is labelled directly or indirectly with a label selected from but not limited to biotin, fluorescein, and an enzyme such as horseradish peroxidase.

The particular label used may not be critical to the present disclosure, so long as it does not interfere with the affinity of the engineered chromodomain for the mLys. In some embodiments, the imaging component is a radionuclide (e.g., ¹⁸F, ¹¹C, ¹³N, ⁶⁴Cu, ⁶⁸Ga, ¹²³I, ¹¹¹In, ⁹⁹mTc, etc.) due to the ease of using such techniques as SPECT, CT, and PET imaging for in vivo detection of engineered chromodomain-mLys complexes and tumor cells. Decision as to appropriate imaging component for agents used in SPECT or PET imaging may also be determined by whether the radionuclide is generated by generator or cyclotron or is a chelator or organic/halide.

A direct labelled probe, as used herein, may be a probe to which a detectable label is attached. Because the direct label is already attached to the probe, no subsequent steps may be required to associate the probe with the detectable label. In contrast, an indirect labeled probe may be one which bears a moiety to which a detectable label is subsequently bound, typically after engineered chromodomain is bound with the target mLys.

In some embodiments, monoclonal antibodies (mAb) which recognize any of the engineered chromodomains of the disclosure may also be made and used to detect the presence of mLys-containing proteins in a sample. mAb's may provide a rapid and simple method of evaluating the engineered chromodomains of the disclosure for their quality. Any suitable methods for the preparation of antibodies may be employed. In some embodiments, methods to produce mAb which specifically recognize the engineered chromodomains of the disclosure are well known to those of skill in the art. In general, peptides are injected in Freund's adjuvant into mice or rabbit. After being injected 9 times over a three-week period, the mice spleens are removed and resuspended in phosphate buffered saline (PBS). The spleen cells may serve as a source of lymphocytes, some of which may be producing antibody of the appropriate specificity. These may then be fused with a permanently growing myeloma partner cell, and the products of the fusion may be plated into several tissue culture wells in the presence of a selective agent such as HAT. The wells may then be screened to identify those containing cells making useful antibody by ELISA. These may then be freshly plated. After a period of growth, these wells may again be screened to identify antibody-producing cells. Several cloning procedures may be conducted until over 90% of the wells contain single clones which are positive for antibody production. From this procedure stable lines of clones may be established which produce the mAb. The mAb may then be purified by affinity chromatography using Protein A or Protein G Sepharose.

In some embodiments, the engineered chromodomains described herein are used as imaging probes for live cell imaging applications. In some embodiments, the live cell imaging applications include visualizing and/or quantifying protein co-localization; 3D imaging of live cells, tissues, model organisms, and small animals; biosensing and protein-protein interactions; visualizing and/or quantifying protein diffusion and kinetics; single molecule tracking; visualizing and/or quantifying co-translational dynamics of nascent peptide chains; visualizing and/or quantifying dynamics of short-lived proteins (e.g., transcription factors, etc.); and tracking the spatiotemporal dynamics of post-translational modifications and protein conformational changes.

In some embodiments, an imaging probe described herein comprises an engineered chromodomain described herein and a tag. When contacting live cells, the imaging probe as disclosed herein can bind to and label methylated protein for imaging the cell to detect and/or quantify the tag. In some embodiments, the imaging probe comprises an engineered chromodomain, an imaging agent, and a linker connecting the engineered chromodomain and the imaging agent. The imaging agent may be at the N-terminus or C-terminus of the protein. In some embodiments the imaging agent is a fluorescent protein, a bioluminescent protein, an inorganic fluorescent probe, or a self-labeling tag. In some embodiments, the engineered chromodomain may further comprise one or more additional tag and/or a sub-cellular localization signal and/or a cell-penetrating domain. In some embodiments, the tag is a hexa-histidine tag. In some embodiments, the tag is a GST-tag.

Non-limiting examples of live cell imaging techniques include transmission light microscopy (e.g., bright field, dark field, phase contrast, differential interference contrast, etc.), fluorescence microscopy, confocal microscopy, multiphoton microscopy, total internal reflection fluorescence microscopy, fluorescence lifetime imaging microscopy, Forster resonance energy transfer microscopy, BRET imaging, fluorescence correlation spectroscopy, single molecule tracking, photo-activation light microscopy, and light sheet microscopy.

### (4) Vectors and Delivery Systems

Provided herein are compositions comprising an engineered chromodomain and a delivery vehicle. The compositions provided herein can be delivered to a target cell, tissue, organ, or subject by any suitable means. In some embodiments, the engineered chromodomain and delivery vehicle can be delivered to a target cell, tissue, organ, or subject by any suitable means.

The engineered chromodomain as described herein can be admixed with a delivery vehicle that permits delivery of the system to the target nucleic acid sequence. In some embodiments, the delivery vehicle comprises a vector, a lipid, a nanoparticle, a plasmid, a virus, a liposome, an extracellular vesicle, or a combination thereof. Additional non-limiting examples of delivery vehicles include an emulsion, a suspension, a liposome, a micelle, an exosome, an endosome, a virus, a vector, a particle, a nanoparticle, a polymer, microcapsules, recombinant cells, cell culture medium, blood, or serum. Specific types of delivery vehicles that can be used in a composition provided herein are further described below.

In some embodiments, the delivery vehicle is a liposome. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core. Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles can also be used as a delivery vehicle.

In some embodiments, the delivery vehicle is a phospholipid. Phospholipids can form a variety of structures other than liposomes when dispersed in water, depending on the molar ratio of lipid to water. At low ratios, the liposomes form. Physical characteristics of liposomes depend on pH, ionic strength, and the presence of divalent cations. Liposomes can show low permeability to ionic and polar substances, but at elevated temperatures undergo a phase transition which markedly alters their permeability. The phase transition involves a change from a tightly packed, ordered structure, known as the gel state, to a loosely packed, less-ordered structure, known as the fluid state. This occurs at a characteristic phase-transition temperature and results in an increase in permeability to ions, sugars, and drugs.

In some embodiments, the delivery vehicle is a nanoparticle. Nanoparticle carriers that specifically target a tissue provided herein may also be used as a pharmaceutically acceptable carrier. In some embodiments, the nanoparticle is a gold nanoparticle, a platinum nanoparticle, an iron-oxide nanoparticle, a lipid nanoparticle, a selenium nanoparticle, a tumor-targeting glycol chitosan nanoparticle (CNP), a cathepsin B sensitive nanoparticle, a hyaluronic acid nanoparticle, a paramagnetic nanoparticle, or a polymeric nanoparticle.

The engineered chromodomains provided herein can be delivered to a cell system using vectors, for example containing polynucleotide sequences encoding a system, a guide polynucleotide, an engineered protein, or a composition provided herein. In some embodiments, a system as described herein can be delivered absent a viral vector. Any vector systems can be used including, but not limited to, plasmid vectors, viral vectors, and oncolytic viral vectors. Furthermore, any of these vectors can comprise one or more transcription factor, transgene, or molecular tag.

In some embodiments, the vectors provided herein are viral vectors. Exemplary viral vectors include, but are not limited to, lentiviral vectors, retroviral vectors, adeno-associated viral vectors (AAV), adenoviral vectors, herpes simplex viral vectors, alpha viral vectors, flaviviral vectors, rhabdoviral vectors, measles viral vectors, Newcastle disease viral vectors, poxviral vectors, picornaviral vectors, and oncolytic viral vectors.

In some embodiments, the viral vector comprises an AAV. AAVs can have one or more of the AAV wild-type genes deleted in whole or part, e.g., the rep and/or cap genes, but retain functional flanking ITR sequences. Functional ITR sequences are necessary for the rescue, replication, and packaging of the AAV virion. The ITRs need not be the wild-type nucleotide sequences, and may be altered, e.g., by the insertion, deletion, or substitution of nucleotides, so long as the sequences provide for functional rescue, replication and packaging. A recombinant AAV vector (rAAV) comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery. In some embodiments, the AAV or the rAAV provided herein comprises a serotype of AAV1, AAV2, AAV3, AAV4, AAV5, AAV6, AAV7, AAV8, AAV9, AAV10, AAV11, AAV12, AAV13, AAVrh10, or any combination thereof.

In some embodiments, the viral vector is a lentiviral vector. In some embodiments, the lentiviral vector is selected from the group consisting of: a human immunodeficiency virus 1 (HIV-1); a human immunodeficiency virus 2 (HIV-2), a visna-maedi virus (VMV) virus; a caprine arthritis-encephalitis virus (CAEV); an equine infectious anemia virus (EIAV); a feline immunodeficiency virus (FIV); a bovine immune deficiency virus (BIV); and a simian immunodeficiency virus (SIV), fragments, derivatives, or variants thereof.

Conventional viral and non-viral based gene transfer methods can be used to introduce polynucleotides encoding for a composition, system, guide polynucleotide, or an engineered protein provided herein to cells (e.g., mammalian cells) and target tissues. Exemplary non-viral vector delivery systems can include DNA plasmids, naked nucleic acid, and nucleic acids complexed with a delivery vehicle such as a liposome or poloxamer. Viral vector delivery systems can also include DNA and RNA viruses, which have either episomal or integrated genomes after delivery to the cell.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. Additional exemplary nucleic acid delivery systems include those provided by AMAXAR Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. Lipofection reagents are sold commercially (e.g., TRANSFECTAM® and LIPOFECTIN®).

Delivery of the engineered chromodomains and systems provided herein can be to cells (ex vivo administration) or target tissues (in vivo administration). Additional methods of delivery include the use of packaging the polynucleotides to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis.

Vectors including viral and non-viral vectors containing nucleic acids encoding a nucleic editing system provided herein can also be administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application, and electroporation. More than one route can be used to administer a particular composition.

In some embodiments, a vector encoding for a composition, or a system provided herein can be shuttled to a cellular nucleus. For example, a vector can contain a nuclear localization sequence (NLS). A vector can also be shuttled by a protein or protein complex. In some embodiments, an engineered chromodomain or a system provided herein can be introduced to a cell or a target tissue by a minicircle vector.

In some embodiments, a vector or a polynucleotide provided herein can be pre-complexed with an engineered protein provided herein prior to electroporation into a cell. An engineered protein that can be used for shuttling can be a nickase or a catalytically dead Cas protein. A nuclease that can be used for shuttling can be a nuclease-competent protein. In some embodiments, an engineered protein herein can be pre-mixed with a guide polynucleotide provided herein an any additional elements (e.g., transgenes or other engineered proteins).

A cell can be transfected with a mutant or chimeric adeno-associated viral vector encoding a system or an engineered chromodomain provided herein. For example, an AAV vector concentration can be from 0.5 nanograms to 50 micrograms.

A system or an engineered chromodomain provided herein can also be introduced to a cell via electroporation techniques. The number of polynucleotides that can be introduced into the cell by electroporation can be varied to optimize transfection efficiency and/or cell viability. In some embodiments, less than about 100 picograms of nucleic acid can be added to each cell sample (e.g., one or more cells being electroporated). In some embodiments, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid can be added to each cell sample (e.g., one or more cells being electroporated). For example, 1 microgram of dsDNA can be added to each cell sample for electroporation. In some embodiments, the amount of nucleic acid (e.g., dsDNA) required for optimal transfection efficiency and/or cell viability can be specific to the cell type. In some embodiments, the amount of nucleic acid (e.g., dsDNA) used for each sample can directly correspond to the transfection efficiency and/or cell viability. The transfection efficiency of cells with any of the nucleic acid delivery platforms described herein, for example, nucleofection or electroporation, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Viral particles, such as AAV, can be used to deliver a viral vector comprising a gene of interest or a transgene into a cell ex vivo or in vivo. In some embodiments, a mutated or chimeric adeno-associated viral vector as disclosed herein can be measured as pfu (plaque forming units). In some embodiments, the pfu of recombinant virus or mutated or chimeric adeno-associated viral vector of the compositions and methods of the disclosure can be about 10⁸ to about 5×10¹⁰ pfu. In some embodiments, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10^{10 3}×10¹⁰, 4×10¹⁰, and 5×10¹⁰ pfu. In some embodiments, a mutated or chimeric adeno-associated viral vector of the disclosure can be measured as vector genomes. In some embodiments, recombinant viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10^{16 1}×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1× 10^{13 1}×10¹⁴, 1×10¹⁵, 1×10¹⁶, 1×10¹⁷, and 1×10¹⁸ vector genomes.

In some embodiments, a mutated or chimeric adeno-associated viral vector of the disclosure can be measured using multiplicity of infection (MOI). In some embodiments, MOI can refer to the ratio, or multiple of vector or viral genomes to the cells to which the nucleic can be delivered. In some embodiments, MOI can refer to the ratio, or multiple of vector or viral genomes to the cells to which the nucleic can be delivered. In some embodiments, the MOI can be 1×10⁶ GC/mL. In some embodiments, the MOI can be 1×10⁵ GC/mL to 1×10⁷ GC/mL. In some embodiments, the MOI can be 1×10⁴ GC/mL to 1×10⁸ GC/mL. In some embodiments, recombinant viruses of the disclosure are at least about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some embodiments, a mutated or chimeric adeno-associated viruses of this disclosure are from about 1×10⁸ GC/mL to about 3×10¹⁴ GC/mL MOI, or are at most about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI.

In some embodiments, a non-viral vector or nucleic acid can be delivered without the use of a mutated or chimeric adeno-associated viral vector and can be measured according to the quantity of nucleic acid. Generally, any suitable amount of nucleic acid can be used with the compositions and methods of this disclosure. In some embodiments, nucleic acid can be at least about 1 μg, 10 μg, 100 μg, 1 μg, 10 μg, 100 μg, 200 μg, 300 μg, 400 μg, 500 μg, 600 μg, 700 μg, 800 μg, 900 μg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some embodiments, nucleic acid can be at most about 1 μg, 10 μg, 100 μg, 1 μg, 10 μg, 100 μg, 200 μg, 300 μg, 400 μg, 500 μg, 600 μg, 700 μg, 800 μg, 900 μg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

Proteins, vectors, plasmids, compositions, systems, engineered proteins, and guide polynucleotides provided herein can be delivered by any suitable method, including transfection, electroporation, liposome delivery, membrane fusion techniques, high velocity DNA-coated pellets, viral infection, and protoplast fusion. The methods used to construct any embodiment of the compositions provided herein include genetic engineering, recombinant engineering, and synthetic techniques.

An engineered chromodomain provided herein can be delivered to a cell by electroporation. Electroporation using, for example, the NEON® Transfection System (Thermo Fisher Scientific) or the AMAXA® Nucleofector (AMAXA® Biosystems) can also be used for delivery of nucleic acids and proteins into a cell. For example, an engineered protein provided herein can be purified and complexed with a suitable guide polynucleotide for delivery into a cell. Electroporation parameters can be adjusted to optimize delivery efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance, and resistance). Application of optimal field strength causes electro-permeabilization through induction of transmembrane voltage, which allows nucleic acids to pass through the cell membrane. In some embodiments, the electroporation pulse voltage, the electroporation pulse width, number of pulses, cell density, and tip type can be adjusted to optimize transfection efficiency and/or cell viability.

### (5) Gene Repression Systems

Provided herein are gene repression systems comprising the engineered chromodomains described herein. In some embodiments, provided herein is a system for repressing gene transcription. In some embodiments, the system for repressing gene transcription comprises a CRISPR-based gene repression system comprising the engineered chromodomains described herein. In some embodiments, the system further comprises a transcriptional regulator, a DNA targeting domain, and a guide nucleic acid. In some embodiments, the guide nucleic acid comprises a targeting sequence complementary to a target nucleic acid sequence of a gene. In some embodiments, the engineered chromodomain enhances the repression of the gene by the transcriptional regulator. In some embodiments, the engineered chromodomain is linked to the transcriptional regulator via a peptide linker. In some embodiments, the peptide linker comprises a sequence at least 80%, at least 85%, at least 90%, or at least 95% sequence identity to an amino acid sequence of SEQ ID NO: 85.

In some embodiments, the guide nucleic acid comprises RNA nucleobases. Examples of nucleobases include, but are not limited to, adenine (A), guanine (G), cytosine (C), and uracil (U). The nucleobase of a nucleotide can be independently selected from a purine, a pyrimidine, a purine, or pyrimidine analog. In an embodiment, the nucleobase can include, for example, naturally occurring and synthetic derivatives of a base. Guide polynucleotides provided herein can comprise non-naturally occurring sequences or engineered sequences.

In some embodiments, the guide nucleic acid comprises a modified nucleobase, a modified nucleotide, or a modified nucleoside. The modified nucleosides and modified nucleotides described herein, which can be incorporated into a target nucleic acid, can include a modified nucleobase. In some embodiments, the nucleobase or the nucleotide provided herein is chemically modified. Nucleobases can be modified or wholly replaced to provide modified nucleosides and modified nucleotides that can be incorporated into a target nucleic acid.

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m C), N4-acetyl-cytidine (act), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k C), a-thio-cytidine, 2′-0-methyl-cytidine (Cm), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f 5 Cm), N4,N4,2′-0-trimethyl-cytidine (m4 2 Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (i A), 2-methyl-adenine (m2A), N6-methyl-adenosine (m6A), 2-methylthio-N6-methyl-adenosine (ms2m6A), N6-isopentenyl-adenosine (16A), 2-methylthio-N6-isopentenyl-adenosine (ms216A), N6-(cis-hydroxyisopentenyl) adenosine (io6A), 2-methylthio-N6-(cis-hydroxyisopentenyl) adenosine (ms2io6A), N6-glycinylcarbamoyl-adenosine (g6A), N6-threonylcarbamoyl-adenosine (16A), N6-methyl-N6-threonylcarbamoyl-adenosine (m6t6A), 2-methylthio-N6-threonylcarbamoyl-adenosine (ms2g6A), N6,N6-dimethyl-adenosine (m6 2A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, a-thio-adenosine, 2′-0-methyl-adenosine (Am), N6,2′-O-dimethyl-adenosine (m6Am), N6-Methyl-2′-deoxyadenosine, N6,N6,2′-0-trimethyl-adenosine (m6 2Am), 1,2′-O-dimethyl-adenosine (i Am), 2′-0-ribosyladenosine (phosphate) (Ar (p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adeno sine.

In some embodiments, the modified nucleobase is a modified guanine. Exemplary nucleobases and nucleosides having a modified guanine include without limitation inosine (I), 1-methyl-inosine (iVI), wyosine (imG), methylwyosine (mimG), 4-demethyl-wyosine (imG-14), isowyosine (imG2), wybutosine (yW), peroxywybutosine (02yW), hydroxy wybuto sine (OHyW), undermodified hydroxy wy buto sine (OHyW*), 7-deaza-guanosine, queuosine (Q),epoxyqueuosine (oQ), galactosyl-queuosine (galQ), mannosyl-queuosine (manQ), 7-cyano-7-deaza-guanosine (preQo), 7-aminomethyl-7-deaza-guanosine (preQO, archaeosine (G+), 7-deaza-8-aza-guanosine, 6-thio-guanosine, 6-thio-7-deaza-guanosine, 6-thio-7-deaza-8-aza-guanosine, 7-methyl-guanosine (m G), 6-thio-7-methyl-guanosine, 7-methyl-inosine, 6-methoxy-guanosine, 1-methyl-guanosine (m′G), N2-methyl-guanosine (m 2 G), N2,N2-dimethyl-guanosine (m 2 2G), N2,7-dimethyl-guanosine (m 2,7G), N2, N2,7-dimethyl-guanosine (m 2,2,7G), 8-oxo-guanosine, 7-methyl-8-oxo-guanosine, 1-meth thio-guanosine, N2-methyl-6-thio-guanosine, N2,N2-dimethyl-6-thio-guanosine, a-thio-guanosine, 2′-0-methyl-guanosine (Gm), N2-methyl-2′-0-methyl-guanosine (m 2″Gm), N2,N2-dimethyl-2′-0-methyl-guano sine (m 2 2Gm), 1-methyl-2′-0-methyl-guanosine (m′Gm), N2,7-dimethyl-2′-0-methyl-guanosine (m″,7Gm), 2′-0-methyl-inosine (Im), 1,2′-0-dimethyl-inosine (m′lm), 06-phenyl-2′-deoxyinosine, 2′-0-ribosylguanosine (phosphate) (Gr (p)), 1-thio-guanosine, 06-methyl-guanosine, 06-Methyl-2′-deoxy guanosine, Z-F-ara-guanosine, and 2′-F-guanosine.

In some embodiments, the modified nucleobase is a modified uracil. Exemplary nucleobases and nucleosides having a modified uracil include without limitation pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 3-methyl-uridine (m 3 U), 5-methoxy-uridine (mo 5 U), uridine 5-oxyacetic acid (cmo 5 U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm 5 se 2 U), 5-carbamoylmethyl-uridine (ncm 5 U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (xcm5U), 1-taurinomethyl-pseudouridine, 5-taurinomethyl-2-thio-uridine (Tm5s2U), 1-taurinomethyl-4-thio-pseudouridine, 5-methyl-uridine (m5U, i.e., having the nucleobase deoxythymine), 1-methyl-pseudouridine (n′y), 5-methyl-2-thio-uridine (m5s2U), 1-methyl-4-thio-pseudouridine (m xi/), 4-thio-1-methyl-pseudouridine, 3-methyl-pseudouridine (m v), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N 1-methyl-pseudouridine, 3-(3-amino-3-carboxypropyl) uridine (acp 3 U), 1-methyl-3-(3-amino-3-carboxypropyl) pseudouridine (acp 3 w), 5-(isopentenylaminomethyl) uridine (inm5U), 5-(isopentenylaminomethyl)-2-thio-uridine (inm5s2U), a-thio-uridine, 2′-0-methyl-uridine (Um), 5,2′-0-dimethyl-uridine (m5Um), 2′-0-methyl-pseudouridine (ymt), 2-thio-2′-0-methyl-uridine (s2Um), 5-methoxycarbonylmethyl-2′-O-methyl-uridine (mem 5Um), 5-carbamoylmethyl-2′-0-methyl-uridine (ncm 5Um), 5-carboxymethylaminomethyl-2′-0-methyl-uridine (cmnm 5 Um), 3,2′-0-dimethyl-uridine (m 3 Um), 5-(isopentenylaminomethyl)-2′-0-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, pyrazolo[3,4-d]pyrimidines, xanthine, and hypoxanthine.

In some embodiments, the modified nucleobase is a modified thymine. In some embodiments, the modified nucleoside is a modified thymidine. Non-limiting examples of modified thymine and thymidine include: 6-(azo) thymine, 3′-azido-3′-deoxythymidine, 2′,3′-didehydro-2′,3′-dideoxythymidine; 1-(2,3-dideoxy-beta-D-glyceropent-2-enofuranosyl) thymine, 3-(2-chloroethyl) thymidine, 3′-fluoro-3′-deoxythymidine, B-L-2′-deoxythymidine, thieno[3,4-d]-pyrimidine T-mimic deoxynucleoside, 1-(2-Deoxy-β-D-threo-pentofuranosyl) thymine, 5-ethynyl-2′-deoxyuridine, bromodeoxyuridine, tritiated thymidine, 5-chlorodeoxyuridine (CldU), 5-iododeoxyuridine (IdU), 2-thiothymidine triphosphate, 5-(α-tert-butylortho-bromobenzyloxy) methyl-2′-deoxyuridine, 5-(α-methylbenzyloxy)methyluracil, and 5-Ethyldeoxyuridine.

Nucleic acids can be modified using various chemistries and modifications. In some embodiments, regular internucleosidic linkages between nucleotides can be altered by mono- or di-thioation of the phosphodiester bonds to yield phosphorothioate esters or phosphorodithioate esters, respectively. Other modifications of the internucleosidic linkages can include amidation or peptide linkers. A ribose sugar can be modified by substitution of the 2′-O moiety with a lower alkyl (C1-4, such as 2′-O-Me), alkenyl (C2-4), alkynyl (C2-4), methoxyethyl(2′-MOE), or other substituent. In some cases, substituents of the 2′ OH group can comprise a methyl, methoxyethyl or 3,3′-dimethylallyl group. In some cases, locked nucleic acid sequences (LNAs), comprising a 2′-4′ intramolecular bridge (such as a methylene bridge between the 2′ oxygen and 4′ carbon) linkage inside the ribose ring, can be applied. Purine nucleobases and/or pyrimidine nucleobases can be modified to alter their properties, for example by amination or deamination of the heterocyclic rings. Many of these modified nucleobases and their corresponding ribonucleosides are available from commercial suppliers. If desired, the guide polynucleotides can contain phosphoramidate, phosphorothioate, and/or methylphosphonate linkages. Several suitable methods can be used to produce nucleic acid molecules and nucleic acids containing modified nucleobases. For example, a guide nucleic acid that contains modified nucleotides can be prepared by transcribing (e.g., in vitro transcription) a DNA that encodes for a guide RNA using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction can contain nucleotides and modified nucleotides, and other components that support the activity of the selected polymerase, such as a suitable buffer, and suitable salts. The incorporation of nucleotide analogs into guide polynucleotides may be engineered, for example, to alter the stability of such molecules or to increase resistance against RNases.

In some embodiments, the DNA targeting domain is an enzymatically inactive nuclease. In some embodiments, the enzymatically inactive nuclease is selected from the group consisting of an enzymatically inactive Cas protein (dCas), a zinc finger domain, or a TAL effector molecule. In some embodiments, the dCas is a transcriptional regulator. In some embodiments, the transcriptional regulator comprises a repressor domain operably linked to a dCas9 protein. Non-limiting examples of enzymatically inactive Cas proteins includes enzymatically inactive Cas9 (dCas9), enzymatically inactive Cas12a (dCpf1), enzymatically inactive Cas12b (dC2cl), enzymatically inactive Cas12c (dC2c3), enzymatically inactive Cas12d (dCasY), enzymatically inactive Cas12e (dCasX), enzymatically inactive Cas12f, enzymatically inactive Cas12g, enzymatically inactive Cas12h, enzymatically inactive Cas12i, enzymatically inactive Cas12j, enzymatically inactive Cas12k, enzymatically inactive Cas121, enzymatically inactive Cas14, and/or enzymatically inactive Cas@.

In some embodiments, the gene repression system as described herein the enzymatic inactive Cas9 proteins are linked to an engineered chromodomain (Cbx) as described herein, and a repressor domain as a fusion protein. In some embodiments, the gene repression system comprises a guide ribonucleic acid (gRNA) for use in repressing a target nucleic acid, inclusive of coding and non-coding regions. A gRNA variant and targeting sequence, and a Cbx-Cas9 fusion protein and a linked repressor domain can form a complex and bind via non-covalent interactions, referred to herein as a ribonucleoprotein (RNP) complex. In some embodiments, the RNP confers advantages in the delivery of the system components to a cell or target gene that comprises a methylated residue for repression of the target gene. In some embodiments, the RNP may provide target specificity to the RNP complex by including a targeting sequence (also referred to as a “spacer”) having a nucleotide sequence that is complementary to a sequence of a target nucleic acid. In some embodiments, the Cbx-dCas9 and linked repressor domain provides site specific activity and is guided to a target site (and further stabilized at a target site) within a target nucleic acid sequence to be modified by virtue of its association with the gRNA. The Cbx-dCas9 protein and linked repressor domain(s) of the RNP complex provides the site-specific activities of the complex such as binding of the target sequence by the Cbx-dCas9 protein and the linked repressor domains provide the repression activity either directly or by the recruitment of other cellular factors.

In some embodiments, the transcriptional repressor domain comprises a Krüppel associated box (KRAB) repressor domain, or a bipartite repressor obtained by fusing the same KRAB domain to Methyl-CpG Binding Protein 2 (MeCP2). In some embodiments, transcriptional repressor domain is a KRAB domain. In some embodiments, the KRAB domain is selected from the group consisting of ZNF343, ZNF337, ZNF334, ZNF215, ZNF519, ZNF485, ZNF214, ZNF33B, ZNF287, ZNF705A, ZNF37A, KRBOX4, ZKSCAN3, ZKSCAN4, ZNF57, ZNF557, ZNF705B, ZNF662, ZNF77, ZNF500, ZNF558, ZNF620, ZNF713, ZNF823, ZNF440, ZNF441, ZNF136, SNRPB, ZNF735, ZKSCAN2, ZNF619, ZNF627, ZNF333, ABCAIIP, PLD5PI, ZNF25, ZNF727, ZNF595, ZNF14, ZNF33A, ZNF101, ZNF253, ZNF56, ZNF720, ZNF85, ZNF66, ZNF722P, ZNF486, ZNF682, ZNF626, ZNF100, ZNF93, ZKSCAN1, ZNF257, ZNF729, ZNF208, ZNF90, ZNF430, ZNF676, ZNF91, ZNF429, ZNF675, ZNF681, ZNF99, ZNF431, ZNF98, ZNF708, ZNF732, SSX2, ZNF721, ZNF726, ZNF730, ZNF506, ZNF728, ZNF141, ZNF723, ZNF302, ZNF484, LINC00960, SSX2B, ZNF718, ZNF74, ZNF157, ZNF790, ZNF565, ZNF705G, VNIR107P, SLC27AS, ZNF737, SSX4, ZNF850, ZNF717, ZNF155, ZNF283, ZNF404, ZNF114, ZNF716, ZNF230, ZNF45, ZNF222, ZNF286A, ZNF624, ZNF223, ZNF284, ZNF790-AS1, ZNF382, ZNF749, ZNF615, ZFP90, ZNF225, ZNF234, ZNF568, ZNF614, ZNF584, ZNF432, ZNF461, ZNF182, ZNF630, ZNF630-AS1, ZNF132, ZNF420, ZNF324B, ZNF616, ZNF471, ZNF227, ZNF324, ZNF860, ZFP28, ZNF470, ZNF586, ZNF235, ZNF274, ZNF446, ZFPI, ZIM3, ZNF212, ZNF766, ZNF264, ZNF480, ZNF667, ZNF805, ZNF610, ZNF783, ZNF621, ZNF8-DT, ZNF880, ZNF213-AS1, ZNF213, ZNF263, ZSCAN32, ZIM2, ZNF597, ZNF786, KRBA1, ZNF460, ZNF8, ZNF875, ZNF543, ZNF133, ZNF229, ZNFS28, SSX1, ZNF81, ZNF578, ZNF862, ZNF777, ZNF425, ZNF548, ZNF746, ZNF282, ZNF398, ZNF599, ZNF251, ZNF195, ZNF181, RBAK-RBAKDN, ZFP37, RN7SL526P, ZNF879, ZNF26, ZSCAN21, ZNF3, ZNF354C, ZNF10 (or KOX1), ZNF75D, ZNF426, ZNF561, ZNF562, ZNF846, ZNF782, ZNF552, ZNF587B, ZNF814, ZNF587, ZNF92, ZNF417, ZNF256, ZNF473, ZFP14, ZFP82, ZNF529, ZNF605, ZFP57, ZNF724, ZNF43, ZNF354A, ZNF547, SSX4B, ZNF585A, ZNFS8SB, ZNF792, ZNF789, ZNF394, ZNF655, ZFP92, ZNF41, ZNF674, ZNFS46, ZNF780B, ZNF699, ZNF177, ZNF560, ZNF583, ZNF707, ZNF808, ZKSCAN5, ZNF137P, ZNF611, ZNF600, ZNF28, ZNF773, ZNF549, ZNF550, ZNF416, ZIKI, ZNF211, ZNF527, ZNF569, ZNF793, ZNF571-AS1, ZNF540, ZNF571, ZNF607, ZNF75A, ZNF205, ZNF175, ZNF268, ZNF354B, ZNF135, ZNF221, ZNF285, ZNF419, ZNF30, ZNF304, ZNF254, ZNF701, ZNF418, ZNF71, ZNF570, ZNF705E, KRBOX1, ZNF510, ZNF778, PRDM9, ZNF248, ZNF845, ZNF525, ZNF765, ZNF813, ZNF747, ZNF764, ZNF785, ZNF689, ZNF311, ZNF169, ZNF483, ZNF493, ZNF189, ZNF658, ZNF564, ZNF490, ZNF791, ZNF678, ZNF454, ZNF34, ZNF7, ZNF250, ZNF705D, ZNF641, ZNF2, ZNF554, ZNF555, ZNF556, ZNF596, ZNF517, ZNF331, ZNF18, ZNF829, ZNF772, ZNF17, ZNF112, ZNF514, ZNF688, PRDM7, ZNF695, ZNF670-ZNF695, ZNF138, ZNF670, ZNF19, ZNF316, ZNF12, ZNF202, RBAK, ZNF83, ZNF468, ZNF479, ZNF679, ZNF736, ZNF680, ZNF273, ZNF107, ZNF267, ZKSCAN8, ZNF84, ZNF573, ZNF23, ZNF559, ZNF44, ZNF563, ZNF442, ZNF799, ZNF443, ZNF709, ZNF566, ZNF69, ZNF700, ZNF763, ZNF433-AS1, ZNF433, ZNF878, ZNF844, ZNF788P, ZNF20, ZNF625-ZNF20, ZNF625, ZNF606, ZNF530, ZNF577, ZNF649, ZNF613, ZNF350, ZNF317, ZNF300, ZNF180, ZNF415, VNIRI, ZNF266, ZNF738, ZNF445, ZNF852, ZKSCAN7, ZNF660, MPRIPP1, ZNF197, ZNF567, ZNF582, ZNF439, ZFP30, ZNF559-ZNF177, ZNF226, ZNF841, ZNF544, ZNF233, ZNF534, ZNF836, ZNF320, KRBA2, ZNF761, ZNF383, ZNF224, ZNF551, ZNF154, ZNF671, ZNF776, ZNF780A, ZNF888, ZNF816-ZNF321P, ZNF321P, ZNF816, ZNF347, ZNF665, ZNF677, ZNF160, ZNF184, ZNF140, ZNF589, ZNF891, ZFP69B, ZNF436, POGK, ZNF669, ZFP69, ZNF684, ZNF124, ZNF496, ZNF656 (or ZIM2) and sequence variants thereof.

In some embodiments, the KRAB repressor domain comprises a KOX1 KRAB domain, a ZIM3 KRAB domain, a ZIM2 KRAB domain, a ZNF554 KRAB domain, a ZNF264 KRAB domain, a ZNF324 KRAB domain, a ZNF354 KRAB domain, a ZFP82 KRAB domain, or a ZNF669 KRAB domain. In some embodiments, the KRAB repressor domain is a KOX1 KRAB domain. In some embodiments, the KRAB repressor domain is a ZIM3 KRAB domain. In some embodiments, the KRAB repressor domain comprises a sequence at least 80%, at least 85%, at least 90%, or at least 95% sequence identity to an amino acid sequence of SEQ ID NOs: 53-54. In some embodiments, the engineered chromodomain linked to the transcriptional regulator via a peptide linker.

Transcriptional repression includes both reversible and irreversible inactivation of gene transcription. In some embodiments, the gene repression system described herein inactivates the transcription a gene, thereby repressing the expression of the gene. Expression level of genes can be measured by real-time quantitative PCR (real-time PCR), western blot analysis, molecular imaging, or flow cytometry. In some embodiments, repression by the systems of the disclosure comprises any detectable decrease in the production of a gene product in cells. In some embodiments, the gene repression system decreases the production of a gene product by at least about 10%, 20%, 30%, 40%, 50%, 60%, 70%, 80%, 90%, 95%, or 99%, or any integer there between, when compared to untreated cells or cells treated with a system that does not include the engineered chromodomain as described herein. In some embodiments, the expression of the gene is reduced by at least 50%, at least 55%, at least 60%, at least 65%, at least 70%, at least 75%, at least 80%, at least 85%, or about 90%. In some embodiments, gene repression results in complete inhibition of gene expression, such that no gene product is detectable. In some embodiments, the repression of transcription by the gene repression systems as described herein is sustained for at least about 1 hour, at least about 2 hours, at least about 3 hours, at least about 4 hours, at least about 5 hours, at least about 6 hours, at least about 7 hours, at least about 8 hours, at least about 1 day, at least about 1 week, at least about 2 weeks, at least about 3 weeks, at least about 1 month, or at least about 3 months, or at least about 6 months when assessed in an in vitro assay, including cell-based assays.

### (6) Kits

Provided herein are kits, wherein the kits comprise: an engineered chromodomain, a library, a nucleic acid or a system provided herein; and packaging and materials therefor. In some embodiments, the kit further comprises a scaffold provided herein. In some embodiments, the kit further comprises a cell-free system. In some embodiments, the kit further comprises a population of cells. In some embodiments, the cells are stored in a cryopreservation medium. In some embodiments, the cryopreservation medium comprises dimethyl sulfoxide (DMSO). In some embodiments, the cryopreservation medium comprises a buffer, an isotonic agent, or an apoptosis inhibitor. Non-limiting examples of buffer elements include citrate, phosphate, succinate, tartrate, fumarate, gluconate, oxalate, lactate, acetate, histidine, and tris. Non-limiting examples of isotonic agents include, for example, citrate, phosphate, succinate, tartrate, fumarate, gluconate, oxalate, lactate, acetate, histidine, and tris. Additional isotonic agents include sodium chloride, potassium chloride, boric acid, sodium borate, mannitol, glycerin, propylene glycol, polyethylene glycol, maltose, sucrose, erythritol, arabitol, xylitol, sorbitol trehalose, and glucose. The apoptosis inhibitor can include, for example, a Rho associated kinase (ROCK) inhibitor, catalase, and zVAD-fmk. In some embodiments, the kits comprise reagents. In some embodiments, the reagents comprise saccharides and saccharide derivatives (e.g., sodium carboxymethyl cellulose and cellulose acetate), detergents, glycols, polyols, esters, buffering agents, alginic acid, and organic solvents.

In some embodiments, a formulation of a composition comprising an engineered chromodomain described herein is prepared in a single container for administration to a cell, a cell-free system, or a subject. In some embodiments, a formulation of a composition provided herein is prepared two containers for administration, separating the guide polynucleotide or polynucleotide encoding the guide polynucleotide and/or the polynucleotide encoding the engineered protein provided herein. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some embodiments, the containers are RNase free.

Provided herein are kits comprising: a first container comprising: a guide polynucleotide or a polynucleotide encoding the guide polynucleotide, wherein the guide polynucleotide comprises: (i) a targeting region that has complementarity to a target nucleic acid; (ii) a protein binding region, wherein the protein binding region comprises a secondary structure that binds to a nuclease; and (iii) a template region, wherein the template region comprises: (1) an insertion template sequence that has complementarity to a target nucleic acid and at least one mismatch nucleobase relative to the target nucleic acid; and (2) a primer region, wherein the primer region comprises: a DNA nucleoside and an RNA nucleoside, and a second container comprising: an engineered protein or a polynucleotide encoding for the engineered protein, wherein the engineered protein comprises: a nickase operably linked to a DNA-dependent DNA polymerase. In some embodiments, a kit provided herein further comprises reagents for nucleic acid amplification, transcription, translation, or nucleic acid isolation. In some embodiments, a kit provided herein further comprises a reporter molecule provided herein.

### (7) Other Applications

In some embodiments, the engineered chromodomains, or a gene that encode one or more of the engineered chromodomains, may be introduced into a mammalian cell line.

In some embodiments, the engineered chromodomains described herein that exhibit super-high affinity to a target mLys site (K_{d} value smaller than about 100 nM) act to mask the target mLys site and may cause severe blocking effects of histone methylation signaling events downstream of the mLys site. Therefore, in some embodiments, the engineered chromodomains described herein that exhibit super-high affinity to a target mLys site serves as an inhibitory reagent of nuclear histone H3 lysine 9 or lysine 27 methylation pathway. Super-high affinity engineered chromodomains derived from different natural chromodomains exhibit distinct sequence recognition specificity. Consequently, a super-high affinity engineered chromodomain, when introduced in a live cell, may block a specific signaling pathway, and may be used as a reagent for investigating physiology of a particular pathway.

In some embodiments, the engineered chromodomains described herein is used as substitutes for an anti-mLys antibody and may be used in research areas where an anti-mLys antibody is used, such as, in some embodiments, Western blots, ELISA, luciferase assay, LUMIER assay, proteomics (enrichment of methylated proteins/peptides), microscopy and so forth.

The engineered chromodomains of the present disclosure that exhibit moderately enhanced affinity (variants that show enhanced affinity compared to the wild type, and in one implementation with a K_{d} value greater than about 100 nM to a target mLys site) may be produced in accordance with the present disclosure. These engineered chromodomains do not have an ability to completely block a mLys site and its downstream signaling, but they may retain inherent sequence recognition specificity of a parent chromodomain to which amino acid substitutions are applied. Therefore, these engineered chromodomains may be used as tracers or biosensors of particular lysine methylation events in cells. To detect the tracer engineered chromodomain in cells, in some embodiments, the engineered chromodomain may be labelled with a probe molecule, as explained above. These biosensors or tracers can be transiently or stably transfected/transformed or delivered using special protein tags as previously described into cells (i.e., mammalian cells, bacteria, single celled organisms, multi-celled organisms, or other similar biological systems).

### EXAMPLES

### Example 1—High Affinity Chromodomain Generation and Analysis

### Synthetic Peptides

Peptides were generated as follows.

All peptides were synthesized by GenScript, desalted, and verified by mass spectrometry. Unless otherwise noted, peptides were modified at the N-terminus with biotin followed by an aminohexanoic acid linker. For fluorescence polarization experiments, H3K9me3 and H3K27me3 peptides, as well as H3K9 and H3K27 non-methylated peptides, were labeled at the N-terminus with fluorescein isothiocyanate (FITC). Peptide sequences are provided in Table 1.

TABLE 1
Sequences of Methylated and non-methylated Peptides

| SEQ ID | | | |
|---|---|---|---|
| NO | Modification Type | Peptide ID | Sequence |
| 1 | Non-modified | H3K9 | ARTKQTARKSTGGKAPR |
| 2 | | H3K14 | TARKSTGGKAPRKQLAT |
| 3 | | H3R17 | KSTGGKAPRKQLATKAA |
| 4 | | H3K27 | KQLATKAARKSAPATGGVK |
| 5 | | H3K36 | KSAPSTGGVKKPHRYKPGT |
| 6 | | H4K20 | GGAKRHRKVLRDNIQ |
| 7 | Methylated | H3K9Mel | ARTKQTARKme1STGGKAPR |
| 8 | | H3K9Me2 | ARTKQTARKme2STGGKAPR |
| 9 | | H3K9Me3 | ARTKQTARKme3STGGKAPR |
| 10 | | H3R17mel | KSTGGKAPRme1KQLATKAA |
| 11 | | H3K27Mel | KQLATKAARKme1SAPATGG |
| | | | VK |
| 12 | | H3K27Me2 | KQLATKAARKme2SAPATGG |
| | | | VK |
| 13 | | H3K27Me3 | KQLATKAARKme3SAPATGG |
| | | | VK |
| 14 | | H3K36Me3 | KSAPSTGGVKme3KPHRYKPG |
| | | | T |
| 15 | | H4K20Me3 | GGAKRHRKme3VLRDNIQ |
| 16 | Acetylated | H3K14Ac | TARKSTGGKacAPRKQLAT |
| 17 | | H3K9Ac | ARTKQTARKacSTGGKAPR |
| 18 | | H3K27Ac | KQLATKAARKacSAPATGGVK |

### Construction of Cbx7-Chromo Library

The phage-displayed Cbx7.V library was constructed by site-directed mutagenesis of a phagemid designed for the display of Cbx7-chromo on the major coat protein P3 of the M13 bacteriophage. A total of 16 residues encompassing two regions involved in methyllysine recognition (region 1: residues 7-12; region 2: positions 32-41) were diversified simultaneously with a soft randomization strategy. Mutagenic oligonucleotides were synthesized to adjust the nucleotide ratio of diversified positions to 70% of the wild-type nucleotide and 10% of each of the other nucleotides. The diversity of the obtained Cbx7.V library was 1.1×10¹⁰. The sequences of the two mutagenic oligonucleotides are provided in Table 2. The numbers indicate nucleotide mixtures of 70% of wild-type nucleotide (represented by 5=A, 6=C, 7=G and 8=T) and 10% of each of the other three nucleotides.

TABLE 2
Sequences of Mutagenic Oligonucleotides

| SEQ ID | | |
|---|---|---|
| NO | Oligonucleotide | Sequence |
| 19 | oGV-Cbx7-R1 | GGCAGCAGCGCAATT(7)(7)(8)(7)(5)(5)(6)(5)(7)(7)(8)(8)(7)(6)(5) |
| | | GTTGAAAGCATTCGTAAAAAACG |
| 20 | oGV-Cbx7-R2 | CGTAAAGGTAAAGTTGAATATCTGGTTAAA(8)(7)G(5)(5)A |
| | | (7)(7)(8)(8)(7)G(6)(6)G(6)(6)G(5)(5)A(8)(5)(8)(5)(7)(6)(5)(6)(6)TG |
| | | GGAACCGGAAGAACATATTCTG |

### Selection of Cbx7 Variants

For selection of Cbx7 variants (Cbx7.Vs), streptavidin was immobilized on 96-well Nunc-Immuno MAXISORP plates (Thermo Fisher Scientific) at 2 μg/ml in PBS pH 7.4, overnight at 4° C. Wells were blocked by addition of 300 μl/well of PBS pH 7.4, 0.5% BSA (PB buffer) and incubation at 25° C. for 1 h. Biotinylated H3K27me3 or H3K27 peptides were added to wells at 2 μg/ml in PBS pH 7.4 and incubated at 25° C. for 30 min. Unbound peptide was removed by washing wells three times with 300 μl/well of PBS pH 7.4, 0.05% Tween-20 (PT buffer). The phage displayed library was cycled through five rounds of binding selections with the immobilized biotinylated H3K27me3 peptide as previously described. To remove form the phage pool clones that bound to nonmethylated targets, the Cbx7-chromo library was pre-incubated for 1 h at 25° C. on plates with an immobilized, biotinylated H3K27 peptide prior to transfer to plates containing the H3K27me3 peptide.

Phage ELISAs were performed to identify positive clones able to bind to the H3K27me3 peptide but not to streptavidin or the unmethylated H3K27 peptide, as described. Amino acid sequences of selected Cbx7.Vs were determined by Sanger DNA sequencing.

### Plasmids for Bacterial and Mammalian Expression of Chromodomains

For BLI and fluorescence polarization experiments, wild-type Cbx chromodomains, Cbx7.V1, Cbx7.V1 back-mutants and Cbx7.VDs gene fragments were amplified by PCR and cloned in frame with a hexa-histidine (His6) tag (SEQ ID NO: 98) and Flag tag into pET21b (EMD Biosciences) using the HiFi DNA Assembly Master Mix (New England Biolabs). Chromodomain boundaries and sequences are listed in Table 3. Sequences were obtained from UniProt, and sequence boundaries used are indicated (residues).

TABLE 3
Chromodomain Boundaries and Sequences

| | Chromodomain | |
|---|---|---|
| SEQ | (UniProt ID, | |
| ID NO | Residues) | Sequence |
| 21 | Cbx1 (P83916, | EEEEYVVEKVLDRRVVKGKVEYLLKWKGFSDEDNTWEPEENL |
| | 17-79) | DCPDLIAEFLQSQKTAHETDK |
| 22 | Cbx2 (Q14781, | GEQVFAAECILSKRLRKGKLEYLVKWRGWSSKHNSWEPEENIL |
| | 8-70) | DPRLLLAFQKKEHEKEVQNR |
| 23 | Cbx3 (Q13185, | EEAEPEEFVVEKVLDRRVVNGKVEYFLKWKGFTDADNTWEPE |
| | 23-81) | ENLDCPELIEAFLNSQK |
| 24 | Cbx4 (O00257, | VGEHVFAVESIEKKRIRKGRVEYLVKWRGWSPKYNTWEPEENI |
| | 6-69) | LDPRLLIAFQNRERQEQLMGY |
| 25 | Cbx5 (P45973, | EEEYVVEKVLDRRVVKGQVEYLLKWKGFSEEHNTWEPEKNLD |
| | 17-78) | CPELISEFMKKYKKMKEGEN |
| 26 | Cbx6 (O95503, | VGERVFAAESIIKRRIRKGRIEYLVKWKGWAIKYSTWEPEENIL |
| | 6-69) | DSRLIAAFEQKERERELYGP |
| 27 | Cbx7 (O95931, | SAIGEQVFAVESIRKKRVRKGKVEYLVKWKGWPPKYSTWEPEE |
| | 4-75) | HILDPRLVMAYEEKEERDRASGYRKRGPK |
| 28 | Cbx8 (Q9HC52, | GERVFAAEALLKRRIRKGRMEYLVKWKGWSQKYSTWEPEENI |
| | 7-69) | LDARLLAAFEEREREMELYGP |
| 29 | Cbx1.VD | EEDEYVVEKVLDRRVVKGKVEYLLKWEGFSDEDNTWEPEENL |
| | | DCPDLIAEFLQSQKTAHETDK |
| 30 | Cbx2.VD | GEDVFAAECILSKRLRKGKLEYLVKWEGWSSKHNSWEPEENIL |
| | | DPRLLLAFQKKEHEKEVQNR |
| 31 | Cbx3.VD | EEAEPEEFVVEKVLDRRVVNGKVEYFLKWKGFTDADNTWEPE |
| | | ENLDCPELIEAFLNSQK |
| 32 | Cbx4.VD | GEDVFAVESIEKKRIRKGRVEYLVKWEGWSPKYNTWEPEENIL |
| | | DPRLLIAFQNRERQEQLMGY |
| 33 | Cbx5.VD | EDEYVVEKVLDRRVVKGQVEYLLKWEGFSEEHNTWEPEKNLD |
| | | CPELISEFMKKYKKMKEGEN |
| 34 | Cbx6.VD | VGDRVFAAESIIKRRIRKGRIEYLVKWEGWAIKYSTWEPEENIL |
| | | DSRLIAAFEQKERERELYGP |
| 35 | Cbx7.VD | SAIGEDVFAVESIRKKRVRKGKVEYLVKWEGWPPKYSTWEPEE |
| | | HILDPRLVMAYEEKEERDRASGYRKRGPK |
| 36 | Cbx8.VD | GEDVFAAEALLKRRIRKGRMEYLVKWEGWSQKYSTWEPEENI |
| | | LDARLLAAFEEREREMELYGP |
| 37 | Cbx2.VD.2 | LSSVGEDVFAAECILSKRLRKGKLEYLVKWEGWSSKHNSWEPE |
| | | ENILDPRLLLAFQKKEHEKEVQNRKRGKRP |
| 38 | Cbx3.VD.2 | EEAEPEEFVVEKVLDRRVVNGKVEYFLKWKGFTDADNTWEPE |
| | | ENLDCPELIEAFLNSQK |
| 39 | Cbx5.VD.2 | EDEYVVEKVLDRRVVKGQVEYLLKWEGFSEEHNTWEPEKNLD |
| | | CPELISEFMKKYKKMKEGEN |
| 40 | Cbx7.VD.2 | ELSAIGEDVFAVESIRKKRVRKGKVEYLVKWEGWPPKYSTWEP |
| | | EEHILDPRLVMAYEEKEERDRASGYRKRGPKP |

For assaying in vitro binding specificity of wild-type chromodomains and Cbx. VDs, the coding sequence of the engineered YY5 firefly luciferase from Photinus pyralis was synthesized as a gene fragment (Integrated DNA Technologies), amplified by PCR, and fused by Gibson assembly to the C-terminus of chromodomains previously cloned into the pET21b vector. The sequences of the above-mentioned constructs are listed in Table 4.

TABLE 4
Sequences of chromodomains cloned into pET21b vector
for expression in E.Coli

| SEQ ID | | |
|---|---|---|
| NO | Construct | Sequence |
| 41 | Cbx1.wt | MSYYHHHHHHGSDYKDDDDKSSGENLYFQGSGGSEEEEYVVEK |
| | | VLDRRVVKGKVEYLLKWKGFSDEDNTWEPEENLDCPDLIAEFLQ |
| | | SQKTAHETDK |
| 42 | Cbx2.wt | MSYYHHHHHHGSDYKDDDDKSSGENLYFQGSGGSGEQVFAAECI |
| | | LSKRLRKGKLEYLVKWKGWSSKHNSWEPEENILDPRLLLAFQKK |
| | | EHEKEVQNR |
| 43 | Cbx3.wt | MSYYHHHHHHGSDYKDDDDKSSGENLYFQGSGGSEEAEPEEFVV |
| | | EKVLDRRVVNGKVEYFLKWKGFTDADNTWEPEENLDCPELIEAF |
| | | LNSQK |
| 44 | Cbx4.wt | MSYYHHHHHHGSDYKDDDDKSSGENLYFQGSGGSGEHVFAVESI |
| | | EKKRIRKGRVEYLVKWRGWSPKYNTWEPEENILDPRLLIAFQNRE |
| | | RQEQLMGY |
| 45 | Cbx5.wt | MSYYHHHHHHGSDYKDDDDKSSGENLYFQGSGGSEEEYVVEKV |
| | | LDRRVVKGQVEYLLKWKGFSEEHNTWEPEKNLDCPELISEFMKK |
| | | YKKMKEGEN |
| 46 | Cbx6.wt | MSYYHHHHHHGSDYKDDDDKSSGENLYFQGSGGSVGERFAAESII |
| | | KRRIRKGRIEYLVKWKGWAIKYSTWEPEENILDSRLIAAFEQKERE |
| | | RELYGP |
| 47 | Cbx7.wt | MSYYHHHHHHGSDYKDDDDKSSGENLYFQGSGGSSAIGEQVFAV |
| | | ESIRKKRVRKGKVEYLVKWKGWPPKYSTWEPEEHILDPRLVMAY |
| | | EEKEERDRASGYRKRGPK |
| 48 | Cbx8.wt | MSYYHHHHHHGSDYKDDDDKSSGENLYFQGSGGSGERVFAAEA |
| | | LLKRRIRKGRMEYLVKWKGWSQKYSTWEPEENILDARLLAAFEE |
| | | REREMELYGP |
| 49 | Cbx1.VD | MSYYHHHHHHGSDYKDDDDKSSGENLYFQGSGGSEEDEYVVEK |
| | | VLDRRVVKGKVEYLLKWEGFSDEDNTWEPEENLDCPDLIAEFLQS |
| | | QKTAHETDK |
| 50 | Cbx2.VD | MSYYHHHHHHGSDYKDDDDKSSGENLYFQGSGGSGEDVFAAECI |
| | | LSKRLRKGKLEYLVKWEGWSSKHNSWEPEENILDPRLLLAFQKKE |
| | | HEKEVQNR |
| 51 | Cbx3.VD | MSYYHHHHHHGSDYKDDDDKSSGENLYFQGSGGSEEAEPEEFVV |
| | | EKVLDRRVVNGKVEYFLKWKGFTDADNTWEPEENLDCPELIEAF |
| | | LNSQK |
| 52 | Cbx4.VD | MSYYHHHHHHGSDYKDDDDKSSGENLYFQGSGGSGEDVFAVESI |
| | | EKKRIRKGRVEYLVKWEGWSPKYNTWEPEENILDPRLLIAFQNRE |
| | | RQEQLMGY |
| 53 | Cbx5.VD | MSYYHHHHHHGSDYKDDDDKSSGENLYFQGSGGSEDEYVVEKV |
| | | LDRRVVKGQVEYLLKWEGFSEEHNTWEPEKNLDCPELISEFMKK |
| | | YKKMKEGEN |
| 54 | Cbx6.VD | MSYYHHHHHHGSDYKDDDDKSSGENLYFQGSGGSVGDRVFAAE |
| | | SIIKRRIRKGRIEYLVKWEGWAIKYSTWEPEENILDSRLIAAFEQKE |
| | | RERELYGP |
| 55 | Cbx7.VD | MSYYHHHHHHGSDYKDDDDKSSGENLYFQGSGGSSAIGEDVFAV |
| | | ESIRKKRVRKGKVEYLVKWEGWPPKYSTWEPEEHILDPRLVMAY |
| | | EEKEERDRASGYRKRGPK |
| 56 | Cbx8.VD | MSYYHHHHHHGSDYKDDDDKSSGENLYFQGSGGSGEDVFAAEA |
| | | LLKRRIRKGRMEYLVKWEGWSQKYSTWEPEENILDARLLAAFEE |
| | | REREMELYGP |

For site-specific recombination into mESCs, coding sequences of Cbx3.wt, Cbx3.VD, Cbx5.wt, Cbx5.VD, Cbx2.VD, Cbx7.VD and their bivalent versions (Cbx2.VD-2x, Cbx7.VD-2x) were synthesized as gene fragments (Integrated DNA Technologies) and cloned into the recombinase-mediated cassette exchange (RCME) targeting vector parbit-v616 by Gibson assembly. The obtained Cbx. VD encoding genes contained a N-terminal acceptor peptide for site-specific biotinylation and were fused in frame with a cassette carrying a nuclear localization signal (NLS) followed by EGFP, an internal ribosome entry site (IRES) and the puromycin-N-acetyltransferase gene. The sequences of the above-mentioned constructs are listed in Table 5.

TABLE 5
Sequences of Chromodomains Used in Live-Cell-imaging and ChIP

| SEQ | | |
|---|---|---|
| ID NO | Construct | Sequence |
| 57 | Cbx2.VD | MAGGLNDIFEAQKIEWHENLYFQGVDEESGSVSGGDLSSVGEDVFA |
| | | AECILSKRLRKGKLEYLVKWEGWSSKHNSWEPEENILDPRLLLAFQK |
| | | KEHEKEVQNRKRGKRPIGGGGSGGGGSAPPKKKRKVGYRDPPVATV |
| | | SKGEELFTGVVPILVELDGDVNGHKFSVSGEGEGDATYGKLTLKFIC |
| | | TTGKLPVPWPTLVTTLTYGVQCFSRYPDHMKQHDFFKSAMPEGYVQ |
| | | ERTIFFKDDGNYKTRAEVKFEGDTLVNRIELKGIDFKEDGNILGHKLE |
| | | YNYNSHNVYIMADKQKNGIKVNFKIRHNIEDGSVQLADHYQQNTPI |
| | | GDGPVLLPDNHYLSTQSALSKDPNEKRDHMVLLEFVTAAGITLGMD |
| | | ELYK |
| 58 | Cbx2.VD- | MAGGLNDIFEAQKIEWHENLYFQGVDEESGSVSGGDLSSVGEDVFA |
| | 2x | AECILSKRLRKGKLEYLVKWEGWSSKHNSWEPEENILDPRLLLAFQK |
| | | KEHEKEVQNRKRGKRPGGGGSEGGGSGGPGSGGEGSAGGGSAGGG |
| | | SLSSVGEDVFAAECILSKRLRKGKLEYLVKWEGWSSKHNSWEPEENI |
| | | LDPRLLLAFQKKEHEKEVQNRKRGKRPIGGGGSGGGGSAPPKKKRK |
| | | VGYRDPPVATVSKGEELFTGVVPILVELDGDVNGHKFSVSGEGEGD |
| | | ATYGKLTLKFICTTGKLPVPWPTLVTTLTYGVQCFSRYPDHMKQHDF |
| | | FKSAMPEGYVQERTIFFKDDGNYKTRAEVKFEGDTLVNRIELKGIDF |
| | | KEDGNILGHKLEYNYNSHNVYIMADKQKNGIKVNFKIRHNIEDGSV |
| | | QLADHYQQNTPIGDGPVLLPDNHYLSTQSALSKDPNEKRDHMVLLE |
| | | FVTAAGITLGMDELYK |
| 59 | Cbx7.VD | MAGGLNDIFEAQKIEWHENLYFQGVDEESGSVSGGDELSAIGEDVFA |
| | | VESIRKKRVRKGKVEYLVKWEGWPPKYSTWEPEEHILDPRLVMAYE |
| | | EKEERDRASGYRKRGPKPIGGGGSGGGGSAPPKKKRKVGYRDPPVA |
| | | TVSKGEELFTGVVPILVELDGDVNGHKFSVSGEGEGDATYGKLTLKF |
| | | ICTTGKLPVPWPTLVTTLTYGVQCFSRYPDHMKQHDFFKSAMPEGY |
| | | VQERTIFFKDDGNYKTRAEVKFEGDTLVNRIELKGIDFKEDGNILGH |
| | | KLEYNYNSHNVYIMADKQKNGIKVNFKIRHNIEDGSVQLADHYQQN |
| | | TPIGDGPVLLPDNHYLSTQSALSKDPNEKRDHMVLLEFVTAAGITLG |
| | | MDELYK |
| 60 | Cbx7.VD- | MAGGLNDIFEAQKIEWHENLYFQGVDEESGSVSGGDELSAIGEDVFA |
| | 2x | VESIRKKRVRKGKVEYLVKWEGWPPKYSTWEPEEHILDPRLVMAYE |
| | | EKEERDRASGYRKRGPKPGGGGSEGGGSGGPGSGGEGSAGGGSAGG |
| | | GSELSAIGEDVFAVESIRKKRVRKGKVEYLVKWEGWPPKYSTWEPE |
| | | EHILDPRLVMAYEEKEERDRASGYRKRGPKPIGGGGSGGGGSAPPKK |
| | | KRKVGYRDPPVATVSKGEELFTGVVPILVELDGDVNGHKFSVSGEGE |
| | | GDATYGKLTLKFICTTGKLPVPWPTLVTTLTYGVQCFSRYPDHMKQ |
| | | HDFFKSAMPEGYVQERTIFFKDDGNYKTRAEVKFEGDTLVNRIELKG |
| | | IDFKEDGNILGHKLEYNYNSHNVYIMADKQKNGIKVNFKIRHNIEDG |
| | | SVQLADHYQQNTPIGDGPVLLPDNHYLSTQSALSKDPNEKRDHMVL |
| | | LEFVTAAGITLGMDELYK |
| 61 | Cbx3.VD | MAGGLNDIFEAQKIEWHENLYFQGVDEESGSVSGGDEEAEPEEFVVE |
| | | KVLDRRVVNGKVEYFLKWKGFTDADNTWEPEENLDCPELIEAFLNS |
| | | QKIGGGGSGGGGSAPPKKKRKVGYRDPPVATVSKGEELFTGVVPILV |
| | | ELDGDVNGHKFSVSGEGEGDATYGKLTLKFICTTGKLPVPWPTLVTT |
| | | LTYGVQCFSRYPDHMKQHDFFKSAMPEGYVQERTIFFKDDGNYKTR |
| | | AEVKFEGDTLVNRIELKGIDFKEDGNILGHKLEYNYNSHNVYIMADK |
| | | QKNGIKVNFKIRHNIEDGSVQLADHYQQNTPIGDGPVLLPDNHYLST |
| | | QSALSKDPNEKRDHMVLLEFVTAAGITLGMDELYK |
| 62 | Cbx5.VD | MAGGLNDIFEAQKIEWHENLYFQGVDEESGSVSGGDEDEYVVEKVL |
| | | DRRVVKGQVEYLLKWEGFSEEHNTWEPEKNLDCPELISEFMKKYKK |
| | | MKEGENIGGGGSGGGGSAPPKKKRKVGYRDPPVATVSKGEELFTGV |
| | | VPILVELDGDVNGHKFSVSGEGEGDATYGKLTLKFICTTGKLPVPWP |
| | | TLVTTLTYGVQCFSRYPDHMKQHDFFKSAMPEGYVQERTIFFKDDG |
| | | NYKTRAEVKFEGDTLVNRIELKGIDFKEDGNILGHKLEYNYNSHNVY |
| | | IMADKQKNGIKVNFKIRHNIEDGSVQLADHYQQNTPIGDGPVLLPDN |
| | | HYLSTQSALSKDPNEKRDHMVLLEFVTAAGITLGMDELYK |
| 63 | Cbx3.wt | MAGGLNDIFEAQKIEWHENLYFQGVDEESGSVSGGDEEAEPEEFVVE |
| | | KVLDRRVVNGKVEYFLKWKGFTDADNTWEPEENLDCPELIEAFLNS |
| | | QKIGGGGSGGGGSAPPKKKRKVGYRDPPVATVSKGEELFTGVVPILV |
| | | ELDGDVNGHKFSVSGEGEGDATYGKLTLKFICTTGKLPVPWPTLVTT |
| | | LTYGVQCFSRYPDHMKQHDFFKSAMPEGYVQERTIFFKDDGNYKTR |
| | | AEVKFEGDTLVNRIELKGIDFKEDGNILGHKLEYNYNSHNVYIMADK |
| | | QKNGIKVNFKIRHNIEDGSVQLADHYQQNTPIGDGPVLLPDNHYLST |
| | | QSALSKDPNEKRDHMVLLEFVTAAGITLGMDELYK |
| 64 | Cbx5.wt | MAGGLNDIFEAQKIEWHENLYFQGVDEESGSVSGGDEEEYVVEKVL |
| | | DRRVVKGQVEYLLKWKGFSEEHNTWEPEKNLDCPELISEFMKKYKK |
| | | MKEGENIGGGGSGGGGSAPPKKKRKVGYRDPPVATVSKGEELFTGV |
| | | VPILVELDGDVNGHKFSVSGEGEGDATYGKLTLKFICTTGKLPVPWP |
| | | TLVTTLTYGVQCFSRYPDHMKQHDFFKSAMPEGYVQERTIFFKDDG |
| | | NYKTRAEVKFEGDTLVNRIELKGIDFKEDGNILGHKLEYNYNSHNVY |
| | | IMADKQKNGIKVNFKIRHNIEDGSVQLADHYQQNTPIGDGPVLLPDN |
| | | HYLSTQSALSKDPNEKRDHMVLLEFVTAAGITLGMDELYK |

### Chromodomain Expression and Purification

E. Coli BL21 (DE3) cells co-transformed with a plasmid for expression of a chromodomain and a plasmid for the expression of the Erv1p and DsbC chaperones were grown in 2YT medium containing 100 μg/ml carbenicillin and 34 μg/ml chloramphenicol at 37° C. with 200 rpm shaking to OD₆₀₀ 0.4. Expression of Erv1p and DsbC was induced with 0.5% (w/vol) arabinose, and the temperature was lowered to 30° C. for 45 min. Expression of chromodomains was induced with 100 μM IPTG at 30° C. for 4 h. Cultures were pelleted and resuspended in 10 ml Lysis Buffer (50 mM Tris-HCl pH 7.8, 1 mM MgCl₂, 1.0% Triton X-100, 1 mM 1,4-Dithiothreitol (DTT), 1 mg/ml lysozyme (Bioshop), 50 U/ml Benzonase (Roche), 1 mM Phenylmethylsulfonyl fluoride (PMSF), and protease inhibitor cocktail (Sigma-Aldrich)) and incubated at 25° C. with gentle agitation for 20 min. Following incubation at 25° C., NaCl was added to the lysate to a final concentration of 300 mM, and the lysate was subjected to two freeze and thaw cycles followed by sonication.

Protein purification was performed by standard methods with Ni-NTA resin (Qiagen). Following protein binding, Ni-NTA resin was washed twice with 30 ml of a high salt wash buffer (50 mM Tris-HCl pH 7.8, 1 M NaCl, 1 mM DTT) to remove DNA bound to chromodomains. Proteins were eluted with a 50-300 mM imidazole buffer gradient. The purity of eluted fractions was determined by SDS-PAGE, and eluted proteins were exchanged into PBS pH 7.4 containing 1 mM DTT (Sigma-Aldrich) by dialysis at 4° C. After elution of chromodomain-luciferase fusions, the buffer was exchanged to TBS pH 8.5 (50 mM Tris-HCl pH 8.5, 150 mM NaCl, 1 mM DTT). For fluorescence polarization (FP) and isothermal titration calorimetry (ITC) experiments, following chromodomains expression and purification, both His6 (SEQ ID NO: 98) and Flag tags were removed by overnight incubation at 4° C. with Tobacco Etch Virus (TEV) protease at a 1:100 protease: substrate molar ratio. After overnight incubation, TEV protease and un-cleaved Cbx proteins were removed by applying the reaction mixture to Ni-NTA resin. Unbound fractions were collected, concentrated using a 3 kDa cut-off Amicon Ultra-4 concentrator (EMD Millipore) and flash frozen to −80° C. Protein concentrations were determined from OD₂₈₀ measurements and calculated using extinction coefficients from ExPASy ProtParam.

### Phage ELISA

Phage ELISA experiments were performed by coating 384-well MAXISORP plates (Thermo Fisher Scientific) with 25 μl/well of 2 μg/ml streptavidin (New England Biolabs, N7021S) in PBS pH 7.4, and incubating them at 4° C. for 16 h. Each well was blocked with 60 μl/well of PBS pH 7.4 containing 0.5% BSA (PB buffer) and incubated at 25° C. for 1 h. Plates were washed four times with 90 μl/well of PBS pH 7.4 containing 0.05% Tween-20 (PT buffer) and incubated with 25 μl/well of biotinylated histone peptides at 2 μg/ml in PBS pH 7.4 for 30 min at 25° C. Plates were incubated with phage stocks previously diluted three-fold in PB buffer containing 0.05% Tween-20 (PBT buffer) at 25° C. for 1 h with gentle agitation. Plates were washed and bound phage was detected using 25 μl/well of an anti-M13-HRP-conjugated antibody (1:3,000 dilution in PT buffer; SinoBiological, 11973-MM05T-H). Following incubation at 25° C. for 30 min with gentle shaking, plates were washed, and binding was assessed by adding 25 μl/well of the 3,3′,5,5′-tetramethylbenzidine (TMB) (Thermo Fisher) chromogenic substrate. Plates were incubated with TMB at 25° C. with gentle shaking for 5-10 min, and the colorimetric reaction was stopped by addition of 25 μl/well of a IM H3PO4 solution. Plates were read spectrophotometrically at 450 nm using a PowerWave XS microplate reader (BioTek).

### Luciferase-Based Assays

For luciferase-based assays, 384-well white plates (PerkinElmer) were coated for 16 h at 4° C. with 4 μg/ml streptavidin (New England Biolabs, N7021S) in TBS pH 8.5 (50 mM Tris-HCl pH 8.5, 150 mM NaCl). Each well was blocked with 60 μl/well of TBS pH 8.5 containing 0.5% BSA (TB buffer) and incubated at 25° C. for 1 h. Plates were washed four times with 90 μl/well of PBS pH 7.4, 0.05% Tween-20 (PT buffer) and incubated with 25 μl/well of biotinylated histone peptides at 5 μg/ml in TB buffer pH 8.5 for 30 min at 25° C. Plates were washed and incubated with Cbx chromodomain-luciferase fusions diluted to 0.1 nM for Cbx. VD or 100 nM for Cbx.wt in TB buffer, 0.05% Tween-20, 1 mM DTT, 5% glycerol (TBT buffer). Plates were incubated for 1 h at 25° C. with gentle shaking, washed six times and luminescence intensity was measured with a Biotek Synergy 5 plate reader (Biotek) using the ONE-Glo™ EX luciferase assay system reagent (Promega). Following background subtraction of the luminescence signal derived from wells coated with streptavidin, the luminescence intensity was normalized to wells with immobilized H3K9me3 peptide. Since no binding was detected for Cbx2.wt with immobilized H3K9me3 peptide, the luminescence intensity measured for Cbx2.wt and Cbx2.VD were normalized against wells immobilized with the H3K27me3 peptide.

### Bio-Layer Interferometry

BLI experiments were performed on an Octet HTX system (ForteBio) using biosensor tips coated with streptavidin to immobilize biotinylated H3K9me3 and H3K27me3 peptides. Binding analysis was performed by analyzing the association of biosensor-immobilized methylated peptides to Cbx7.V1 variants in solution at 5 or 1 μM. Binding assays were performed at 25° C. in BLI reaction buffer (25 mM HEPES pH 7.0, 150 mM NaCl, 1 mg/ml BSA, 0.01% Tween-20). Dissociation constants (KD) were obtained by fitting the response wavelength shifts in the steady-state regions with the Langmuir binding model.

### Fluorescence Polarization Binding Assays

Fluorescence polarization (FP) assays were performed in a 20 μl volume at a constant histone peptide concentration of 0.5 nM. Binding measurements were performed in FP buffer (25 mM HEPES pH 7.5, 150 mM NaCl, 1 mM DTT, 0.01% TritonX-100) by mixing in a 384-well black plate (Corning) 0.5 nM FITC-labeled histone peptides with serial dilutions of wild-type Cbx chromodomains ranging from 200-0.2 μM. For affinity determination of Cbx7.V1 and all Cbx. VDs, chromodomains were serially diluted from 50 μM to 50 nM. Samples were incubated for 30 min at 25° C. prior to data acquisition. Fluorescence polarization was measured using an excitation filter of 485 nm and an emission filter of 530 nm with a Synergy Neo2 Multi-Mode Assay Microplate Reader (Biotek). Dissociation constants were determined using Prismv.9.3 (GraphPad Software Inc) with a one-site total binding model.

### Isothermal Titration Calorimetry

ITC experiments were performed on a MicroCal PEAQ-ITC Automated (Malvern Instruments) by titrating H3K9me3 and H3K27me3 peptides (between 1.2 and 2 mM from the syringe, depending on the experiment) into Cbx7.V1 (between 70 and 112 μM in the cell, depending on the experiment) at 25° C. in PBS pH 7.4 containing 1 mM TCEP ((tris (2-carboxyethyl) phosphine)). Dissociation constants were determined using the one-site binding model supplied in MicroCal PEAQ-ITC analysis software (version 1.1.0.1262) with the fitted offset control. Data are the average from three independent experiments ±1 SD.

### Thermal Stability Measurements

For measuring thermal stability, 40 μM Cbx4.wt or Cbx4.VD protein sample in PBS pH 7.4 in a final volume of 30 μL was incubated at 25, 40, 50, 70, or 90° C. for 3 min and then cooled to 10° C. at 3° C./s in a Bio-Rad C1000 Thermal Cycler. Samples were then spun at 17,000×g at 4° C. for 30 min to remove aggregates, and the supernatant was analyzed by SDS-PAGE. Gels were stained with Coomassie blue and band intensities were quantified by densitometry using a Gel Doc XR imager and Image Lab 3.0 software (Bio-Rad). Percent soluble chromodomain was calculated as×100 (band intensity at the indicated temperature/band intensity of samples heated at 25° C.).

### Cell Culture

HEK293T (ATCC), HeLa (ATCC), and HEK293T SV40-EGFP reporter cells (a gift from the Taipale laboratory, University of Toronto) were grown in Dulbecco's Modified Eagle Medium (DMEM) (Life Technologies) containing 10% (vol/vol) fetal bovine serum (FBS, Sigma-Aldrich) at 37° C. with 5% CO2, whereas U20S cells (ATCC) were grown at 37° C. with 5% CO₂ in McCoy's 5a Medium Modified (ATCC) containing 10% FBS. Mouse embryonic stem cells (mESCs) (HA36CB1, 129-C57Bl/6, ATCC) were grown on feeder cells or 0.2% gelatine coated dishes, in DMEM supplemented with 15% fetal calf serum (Invitrogen), 1× nonessential amino acids (Invitrogen), 1 mM L-glutamine, LIF, and 0.001% β-mercaptoethanol. Mouse embryonic stem cells were stably integrated with Cbx. VD-encoding genes. Briefly, Cbx. VDs cloned into the recombinase-mediated cassette exchange (RCME) targeting vector parbit-v616 were co-transfected with a Cre recombinase-encoding plasmid (1:0.6 plasmid DNA ratio) into RCME competent mESCs that stably expressed E. coli biotin ligase (BirA) for site-specific biotinylation of Cbx. VDs. Transfected cells were selected for 4 days with 3 mM ganciclovir, followed by a second selection round with 2 mM puromycin for 2 days. The Eed-KO cell line was generated and cultured as previously described. All cell lines were routinely tested for mycoplasma contamination.

### Plasmids for Lentivirus Production

Genes encoding Cbx-KRAB domain fusions were synthesized as gene fragments (Integrated DNA Technologies) and cloned with the Gateway BP Clonase II system (Thermo Fisher, 11789020) into the Gateway Entry vector pDONR221 (Thermo Fisher) according to the manufacturer's protocols. Cbx-KRAB domain fusions cloned into pDONR221 were then transferred, via Gateway LR Clonase II system (Thermo Fisher, 11791020), into the pLX303-dCas9 vector. The pLX303-dCas9 vector encoded a Streptococcus pyogenes dCas9 with two C-terminal and one N-terminal SV40 nuclear localization signals. Sequences of all dCas9-repressors constructed are listed in Table 6.

TABLE 6
Sequences of dCas9-repressors

| SEQ | | |
|---|---|---|
| ID NO | Construct | Construct sequence |
| 65 | KOX1-dCas9 | MDAKSLTAWSRTLVTFKDVFVDFTREEWKLLDTAQQIVYRNV |
| | | MLENYKNLVSLGYQLTKPDVILRLEKGEEPWLV |
| 66 | ZIM3-dCas9 | MNNSQGRVTFEDVTVNFTQGEWQRLNPEQRNLYRDVMLENYS |
| | | NLVSVGQGETTKPDVILRLEQGKEPWLEEEEVLGSGRAEKNGDI |
| | | GGQIWKPKDVKESLS |
| 67 | Cbx1.wt- | MEEEEYVVEKVLDRRVVKGKVEYLLKWKGFSDEDNTWEPEEN |
| | KOX1-dCas9 | LDCPDLIAEFLQSQKTAHETDKSGGGSGGSGSMDAKSLTAWSR |
| | | TLVTFKDVFVDFTREEWKLLDTAQQIVYRNVMLENYKNLVSL |
| | | GYQLTKPDVILRLEKGEEPWLVSGGGSGSSGS |
| 68 | Cbx1.VD- | MEEDEYVVEKVLDRRVVKGKVEYLLKWEGFSDEDNTWEPEEN |
| | KOX1-dCas9 | LDCPDLIAEFLQSQKTAHETDKSGGGSGGSGSMDAKSLTAWSR |
| | | TLVTFKDVFVDFTREEWKLLDTAQQIVYRNVMLENYKNLVSL |
| | | GYQLTKPDVILRLEKGEEPWLVSGGGSGGSGS |
| 69 | Cbx2.wt- | MGEQVFAAECILSKRLRKGKLEYLVKWKGWSSKHNSWEPEEN |
| | KOX1-dCas9 | ILDPRLLLAFQKKEHEKEVQNRSGGGSGGSGSMDAKSLTAWS |
| | | RTLVTFKDVFVDFTREEWKLLDTAQQIVYRNVMLENYKNLVSL |
| | | GYQLTKPDVILRLEKGEEPWLVSGGGSGGSGS |
| 70 | Cbx2.VD- | MGEDVFAAECILSKRLRKGKLEYLVKWEGWSSKHNSWEPEENI |
| | KOX1-dCas9 | LDPRLLLAFQKKEHEKEVQNRSGGGSGGSGSMDAKSLTAWSR |
| | | TLVTFKDVFVDFTREEWKLLDTAQQIVYRNVMLENYKNLVSL |
| | | GYQLTKPDVILRLEKGEEPWLVSGGGSGGSGS |
| 71 | Cbx3.wt- | MEEAEPEEFVVEKVLDRRVVNGKVEYFLKWKGFTDADNTWEP |
| | KOX1-dCas9 | EENLDCPELIEAFLNSQKGGGSGGSGSMDAKSLTAWSRTLVTF |
| | | KDVFVDFTREEWKLLDTAQQIVYRNVMLENYKNLVSLGYQLT |
| | | KPDVILRLEKGEEPWLVSGGGSGGSGS |
| 72 | Cbx3.VD- | MEEAEPDEFVVEKVLDRRVVNGKVEYFLKWEGFTDADNTWEP |
| | KOX1-dCas9 | EENLDCPELIEAFLNSQKSGGGSGGSGSMDAKSLTAWSRTLVT |
| | | FKDVFVDFTREEWKLLDTAQQIVYRNVMLENYKNLVSLGYQL |
| | | TKPDVILRLEKGEEPWLVSGGGSGGSGS |
| 73 | Cbx3.VD-2x- | MEEAEPDEFVVEKVLDRRVVNGKVEYFLKWEGFTDADNTWEP |
| | KOX1-dCas9 | EENLDCPELIEAFLNSQKSGGGSGGSGSMEEAEPDEFVVEKVL |
| | | DRRVVNGKVEYFLKWEGFTDADNTWEPEENLDCPELIEAFLNS |
| | | QKSGGGSGGSGSMDAKSLTAWSRTLVTFKDVFVDFTREEWKL |
| | | LDTAQQIVYRNVMLENYKNLVSLGYQLTKPDVILRLEKGEEPW |
| | | LVSGGGSGGSGS |
| 74 | Cbx3.VD- | MEEAEPDEFVVEKVLDRRVVNGKVEYFLKWEGFTDADNTWEP |
| | ZIM3-dCas9 | EENLDCPELIEAFLNSQKSRSSGGGSGGSGSMNNSQGRVTFED |
| | | VTVNFTQGEWQRLNPEQRNLYRDVMLENYSNLVSVGQGETTK |
| | | PDVILRLEQGKEPWLEEEEVLGSGRAEKNGDIGGQIWKPKDVK |
| | | ESLSGGGSGGSGS |
| 75 | Cbx5.wt- | MEEEYVVEKVLDRRVVKGQVEYLLKWKGFSEEHNTWEPEKNL |
| | KOX1-dCas9 | DCPELISEFMKKYKKMKEGENSGGGSGGSGSMDAKSLTAWSR |
| | | TLVTFKDVFVDFTREEWKLLDTAQQIVYRNVMLENYKNLVSL |
| | | GYQLTKPDVILRLEKGEEPWLVSGGGSGGSGS |
| 76 | Cbx5.VD- | MEDEYVVEKVLDRRVVKGQVEYLLKWEGFSEEHNTWEPEKNL |
| | KOX1-dCas9 | DCPELISEFMKKYKKMKEGENSGGGSGGSGSMDAKSLTAWSR |
| | | TLVTFKDVFVDFTREEWKLLDTAQQIVYRNVMLENYKNLVSL |
| | | GYQLTKPDVILRLEKGEEPWLVSGGGSGGSGS |
| 77 | Cbx5.VD-2x- | MEDEYVVEKVLDRRVVKGQVEYLLKWEGFSEEHNTWEPEKNL |
| | KOX1-dCas9 | DCPELISEFMKKYKKMKEGENSGGGSGGSGSMEDEYVVEKVL |
| | | DRRVVKGQVEYLLKWEGFSEEHNTWEPEKNLDCPELISEFMKK |
| | | YKKMKEGENSGGGSGGSGSMDAKSLTAWSRTLVTFKDVFVD |
| | | FTREEWKLLDTAQQIVYRNVMLENYKNLVSLGYQLTKPDVILR |
| | | LEKGEEPWLVSGGGSGGSGS |
| 78 | Cbx6.wt- | MVGERFAAESIIKRRIRKGRIEYLVKWKGWAIKYSTWEPEENIL |
| | KOX1-dCas9 | DSRLIAAFEQKERERELYGPSGGGSGGSGSMDAKSLTAWSRTL |
| | | VTFKDVFVDFTREEWKLLDTAQQIVYRNVMLENYKNLVSLGY |
| | | QLTKPDVILRLEKGEEPWLVSGGGSGGSGS |
| 79 | Cbx6.VD- | MVGDRVFAAESIIKRRIRKGRIEYLVKWEGWAIKYSTWEPEENI |
| | KOX1-dCas9 | LDSRLIAAFEQKERERELYGPSGGGSGGSGSMDAKSLTAWSRT |
| | | LVTFKDVFVDFTREEWKLLDTAQQIVYRNVMLENYKNLVSLG |
| | | YQLTKPDVILRLEKGEEPWLVSGGGSGGSGS |
| 80 | Cbx7.wt- | MSAIGEQVFAVESIRKKRVRKGKVEYLVKWKGWPPKYSTWEP |
| | KOX1-dCas9 | EEHILDPRLVMAYEEKEERDRASGYRKRGPKSGGGSGGSGSM |
| | | DAKSLTAWSRTLVTFKDVFVDFTREEWKLLDTAQQIVYRNVM |
| | | LENYKNLVSLGYQLTKPDVILRLEKGEEPWLVSGGGSGGSGS |
| 81 | Cbx7.VD- | MSAIGEDVFAVESIRKKRVRKGKVEYLVKWEGWPPKYSTWEP |
| | KOX1-dCas9 | EEHILDPRLVMAYEEKEERDRASGYRKRGPKSGGGSGGSGSM |
| | | DAKSLTAWSRTLVTFKDVFVDFTREEWKLLDTAQQIVYRNVM |
| | | LENYKNLVSLGYQLTKPDVILRLEKGEEPWLVSGGGSGGSGS |
| 82 | Cbx7.VD-2x- | MSAIGEQVFAVESIRKKRVRKGKVEYLVKWKGWPPKYSTWEP |
| | KOX1-dCas9 | EEHILDPRLVMAYEEKEERDRASGYRKRGPKSGGGSGGSGSM |
| | | SAIGEQVFAVESIRKKRVRKGKVEYLVKWKGWPPKYSTWEPEE |
| | | HILDPRLVMAYEEKEERDRASGYRKRGPKSGGGSGGSGSMDA |
| | | KSLTAWSRTLVTFKDVFVDFTREEWKLLDTAQQIVYRNVMLE |
| | | NYKNLVSLGYQLTKPDVILRLEKGEEPWLVSGGGSGGSGS |
| 83 | Cbx8.wt- | MGERVFAAEALLKRRIRKGRMEYLVKWKGWSQKYSTWEPEE |
| | KOX1-dCas9 | NILDARLLAAFEEREREMELYGPSGGGSGGSGSMDAKSLTAW |
| | | SRTLVTFKDVFVDFTREEWKLLDTAQQIVYRNVMLENYKNLVS |
| | | LGYQLTKPDVILRLEKGEEPWLVSGGGSGGSGS |
| 84 | Cbx8.VD- | MGEDVFAAEALLKRRIRKGRMEYLVKWEGWSQKYSTWEPEE |
| | KOX1-dCas9 | NILDARLLAAFEEREREMELYGPSGGGSGGSGSMDAKSLTAW |
| | | SRTLVTFKDVFVDFTREEWKLLDTAQQIVYRNVMLENYKNLVS |
| | | LGYQLTKPDVILRLEKGEEPWLVSGGGSGGSGS |
| 85 | Linker | SGGGSGGSGS |

### Lentivirus Production and Generation of Stable Cell Lines

Lentivirus was produced by transiently transfecting low passaged HEK293T cells seeded in 6-well plates, 2 ml/well, at 0.3×10⁶ cells/ml. Cells were transfected in Opti-MEM (Thermo Fisher) with 1.25 μg of each chromodomain-encoding plasmid, 900 ng of the lentiviral packaging plasmid psPAX2 and 150 ng of envelope plasmid pVSV-G using Lipofectamine 2000 (Thermo Fisher) according to the manufacturer's instructions. Six hours post transfection, media was replaced with DMEM containing 10% FBS and 11% BSA. Viral particles were harvested 48-72 h post transfection and filtered through a 0.45 μm filter. For generation of cells stably expressing Cbx-KRAB-dCas9 fusions, HEK293T, HeLa, U20S and HEK293T SV40-EGFP reporter cells were seeded in six-well plates at 0.3×10⁶ cells/ml and transduced overnight in complete media containing 10% FBS and 8 μg/ml hexadimethrine bromide (Polybrene, Sigma-Aldrich) at high multiplicity of infection with viral particles containing each repressor-encoding plasmid. Following overnight incubation, lentiviral particles were removed, and transduced cells were selected with complete media containing 10% FBS and blasticidin. For selection of stably transfected HEK293T and HEK293T SV40-EGFP, cells were treated with 6 μg/ml blasticidin for 6 days. HeLa cells transduced with Cbx-KRAB-dCas9 fusions were selected by culturing cells for 4 days in DMEM containing 10% FBS and 4 μg/ml blasticidin. Transduced U20S cells were selected by culturing cells in McCoy's 5a Medium Modified containing 10% FBS and 5 μg/ml blasticidin for 4 days. For CD81, APRC2 and ERK repression experiments, lentivirus containing a plasmid encoding one guide RNA (gRNA) targeting CD81, two gRNA-encoding plasmids for APRC2 and ERK were produced by transiently transfecting low passaged HEK293T cells with 900 ng of the psPAX2 plasmid, 150 ng of the pVSV-G vector and 1.25 μg of the pLCKO vector expressing the U6-driven CD81, APRC2 and ERK gRNAs. gRNA sequences are listed in Table 7. Transfections were performed using Lipofectamine 2000 (Thermo Fisher) according to the manufacturer's instructions. Viral particles were harvested 72 h post transfection, filtered through a 0.45 μm filter and used to infect overnight HEK293T, HeLa and U20S cells previously transduced with CRISPRi repressors. Following overnight incubation, lentiviral particles were removed, and transduced cells were selected using complete media containing puromycin. Transduced HEK293T and HeLa cells were grown for 3 days in DMEM containing 10% FBS and 2 μg/ml or 6 μg/ml puromycin, respectively. To isolate transduced U20S, cells were grown in McCoy's 5a Medium containing 10% FBS and 5 μg/ml puromycin for 4 days. HEK293T, HeLa and U20S cells stably expressing both gRNAs and CRISPRi repressors were maintained for 4 days before flow cytometry and western blot analysis.

TABLE 7
Sequences of gRNAs

| SEQ ID NO | CRISPRi target | sgRNA sequence |
|---|---|---|
| 86 | SV40 | GAAAGTCCCCAGGCTCCCCAGC |
| 87 | CD81 | GCCTGGCAGGATGCGCGGTG |
| 88 | ERK1-1 | GGGAGCCCCGTAGAACCGAG |
| 89 | ERK1-2 | CACCGCCCTCCTCCCCACGG |
| 90 | ARPC2-1 | TGTCGGTGAAGCGGCAGTGG |
| 91 | ARPC2-2 | CAGGCGGGTTCAGGCTTCGG |

### Time Course Assessment of EGFP Repression

HEK293T-SV40 EGFP cells were seeded into glass bottom Incucyte® Imagelock 96-well plates (Essen Bioscience) at 0.4×10⁵ cells (100 μl/well). Cells were transfected with 0.3 μg plasmids encoding Cbx3.VDKOX1-dCas9, KOX1-dCas9 and dCas9 only using Lipofectamine 2000 (Thermo Fisher) according to the manufacturer's instructions. Twelve hours post transfection, media was replaced, and plates were incubated for 168 h in the Incucyte® live-cell analysis system (Essen Bioscience) with automatic image acquisition every 3 h.

### Flow Cytometry

To determine expression levels of Cbx-EGFP fusions integrated into mESC genome, cells were resuspended in DPBS and stained with LIVE/DEAD Fixable Near-IR Dead Cell Stain (Invitrogen, L34975) to assess cell viability. Cells were analyzed on a FACSCanto flow cytometer (BD Biosciences) using the EGFP (Alexa Fluor 488-A), and live/dead (APCCy7A) filters to detect live cells expressing EGFP.

To assess the repressive potential of each Cbx-KRAB-dCas9 fusion, stably transfected HEK293T SV40-EGFP reporter cells were harvested by trypsinization, washed once with ice-cold DPBS, and resuspended in PBS containing 1% BSA and 0.1% NaN3 (FACS-A buffer) at 2.5×10⁶ cells/ml.

To determine the correlation between methylation and chromodomain-mediated enhanced gene repression, cells were treated for 24 h with 10 μM chaetocin (StemCell technologies) dissolved in DMSO prior to flow cytometry analysis. As a control, cells were incubated for 24 h with DMSO in absence of the histone lysine methyltransferase inhibitor.

For CD81 repression analysis, HEK293T cells stably expressing CD81 gRNA and CRISPRi repressors were harvested by trypsinization, washed once with ice-cold DPBS, and resuspended in FACS-A buffer at 2.5×10⁶ cells/ml. Labelling of cell-surface CD81 was performed by incubating 2.5×10⁵ cells with 25 μl of the phycoerythrin (PE)-conjugated mouse anti human CD81 antibody (clone JS-81, BDBiosciences) on ice for 30 min. Antibody excess was removed by centrifugation at 390×g, 4° C. for 3 min. Cells were washed three times with FACS-A buffer and then resuspended in the same buffer at 2.5×10⁶ cells/ml. Cells were analyzed on a LSR Fortessa X20 flow cytometer (Becton Dickinson), and data were analyzed with FlowJo Software (version 10.1, FlowJo, LLC).

### Live-Cell Imaging

Live-cell imaging experiments were performed by seeding mESCs expressing Cbx. VDs (0.2× 105 cells) on a 0.2% gelatin-coated 35-mm chambered coverslip (Ibidi, 80826) 1 day before imaging. Nuclei were stained with Hoechst 33342 (Invitrogen, 62249) for 10 min, washed twice with DPBS and covered with embryonic stem cell medium containing DMEM without phenol red (Invitrogen, 31053028). Images were acquired with a P5 inverted confocal laser scanning microscope (Leica) equipped with a climate chamber and an argon laser, using the GFP (excitation BP 470/40, emission BP 525/50) and N3 (excitation BP 546/12, emission BP 600/400) filters. Image analysis was performed using Fiji software (version 2.0.0) 51. Appropriate single z planes were selected for image display. Chromatin immunoprecipitation sequencing ChIP samples were collected from two biological replicates (independent mESC cell lines). For chromatin extraction, 30-50× 10⁶ cells were trypsinized, washed once in PBS and fixed with 1% formaldehyde for 8 min at 25° C., followed by reaction quenching with addition of glycine to a final concentration 0.12 M and incubation on ice for 10 min. Cells were washed twice in 10 ml ice-cold PBS, followed by centrifugation at 680×g for 5 min. Cells were resuspended and incubated for 10 min in 10 ml Paro Rinse 1 buffer (10 mM Tris pH 8.0, 10 mM EDTA, 0.5 mM EGTA) on ice, followed by centrifugation at 680×g for 5 min. Cells were resuspended in 10 ml of Paro Rinse 2 buffer (10 mM Tris pH 8.0, 0.25% Triton X-100, 1 mM EDTA, 0.5 mM EGTA and 200 mM NaCl), filtered for three times through a prechilled G26 syringe, and incubated for 10 min on ice followed by centrifugation at 680×g for 5 min. Final cell lysis was performed with Mnase Digest buffer (20 mM Tris pH 8.0, 5 mM MgCl₂, 1 mM CaCl₂), 10 mM NaCl, 0.25 M Sucrose, 1% Triton X-100, supplemented with protease inhibitor cocktail mix (Roche, 11836170001)) in a concentration-dependent volume (16,500 cells per microliter) for 0.5 to 1 h on ice. Cross-linked chromatin was subjected to digestion with MNase (New England Biolabs) by adding 50 units of MNase per 1 ml of buffer and incubating the reaction at 37° C. for 20 min with repeated stirring. The MNase digestion was stopped by adding 10× Stop buffer to a final concentration of 150 mM NaCl, 100 mM EDTA, 200 mM EGTA on ice and SDS to 0.1% final concentration. MNase-digested chromatin was subjected to sonication (four cycles of 32″ON/50″OFF) in a Bioruptor Pico instrument (Diagenode) according to the manufacturer's instructions. Sonicated chromatin was centrifuged at 12,000×g for 10 min at 4° C. and supernatant was used for further steps. For biotin ChIP-seq experiments, 100 μg chromatin extracted from mESCs expressing biotinylated Cbx. VDs were applied to 30 μl of streptavidin coated M280 magnetic beads (Thermo Fisher) previously blocked for 1 h at 4° C. with 1% cold fish skin gelatin (Sigma-Aldrich, G7041) and 100 ng yeast tRNA (Thermo Fischer, 15401011) supplemented with protease inhibitor cocktail mix (Roche). Chromatin was incubated with beads at 4° C., overnight with end-over-end rotation. Following overnight incubations, beads were washed, and chromatin was eluted. ChIP-seq libraries were prepared using the NEBNext Ultra II DNA library prep Kit for Illumina (New England Biolabs, E7645L) according to the manufacturer's instructions. Samples tagged with unique index barcodes were combined at equal molar ratios and sequenced as pools on a NovaSeq 6000 sequencing system (Illumina) according to Illumina standards with 100 base-pair single-end sequencing. Library demultiplexing was performed following Illumina standards.

### Genomic Data Analysis

Adaptor sequences were removed using Trim Galore (https://github.com/FelixKrueger/TrimGalore). Trimmed reads were mapped to the mm9 mouse genome reference using Bowtie2 version 2.3.5.1 using the —no-unal and —very-sensitive options. Alignments were stored as BAM files, low-quality reads (excluding reads with mapping quality <40) were filtered out using SAMtools 1.11, and identical reads from PCR duplicates removed with Picard Toolkit (version: 2.23.9, https://broadinstitute.github.io/picard/). BAM files were then converted to bigWig files for visual inspection in genome browsers, and GC-bias was checked and corrected using the correct GCBias module of deepTools 3.5.0. Using BamTools 2.5.1, ChIP replicates for each construct were merged, sorted, and a bigWig file was generated at 100 base-pair resolution excluding mm9-blacklisted regions using the BamCoverage module of deepTools. DeepTools was also used to normalize all ChIP samples collected from Cbx. VD-expressing cells (Cbx. VD ChIP) to a GFP-control sample, plot correlation matrices, and plot average profiles around antibody-specific peaks for histone modifications. In brief, a bigWig file was generated based on two BAM files (Cbx. VD ChIP and GFP control ChIP) that were compared to each other while being simultaneously normalized for sequencing depth (using BamCompare-normalize, using reads per million mapped reads and excluding blacklisted regions). To obtain genome-wide 1-kb intervals, the entire genome was partitioned into 1-kb-sized bins. Intervals overlapping with ENCODE blacklisted regions were removed to reduce artefacts due to annotation errors. MultiBigwigSummary was used to compute the average scores for each of the bigWig files in every genomic interval. To detect antibody-specific peaks for histone modifications, previously published ChIP-seq samples were used and MACS2 was applied with input chromatin as a background signal using the following parameters: —broad, -g mm, —broad-cutoff 0.05. Finally, compute-Matrix was used to calculate the coverage per defined genome regions (antibody-specific peaks for H3K27me3 and H3K9me3) and to prepare an intermediate file that could be used with plotProfiles.

### Immunoblotting

Whole cell lysates (WCL) of cells stably expressing dCas9-fused repressors were prepared in 50 mM Tris-HCl, pH 7.5, 150 mM NaCl, 1 mM EDTA, 1% Triton X-100, 1 mM DTT, and Complete protease inhibitor cocktail (Roche). In all, 15 μg of each WCL were loaded onto a reducing SDS-PAGE and following electrophoretic separation, proteins were transferred onto Immobilion-P polyvinylidene difluoride (PVDF) membranes (Merk Millipore) using the Trans-Blot Turbo Transfer system (BioRad). Blots were blocked overnight with a Tris-HCl pH 7.5, 150 mM NaCl, 5% skimmed milk solution (TBSM) at 4° C., with gentle agitation. Blots were incubated with the following primary antibodies: 1 μg/ml anti-CRISPR-Cas9 (clone 7A9-3A3, Abcam) and 0.44 μg/ml anti-GAPDH (clone FF26A, Abcam) in TBSM buffer containing 0.05% Tween-20 at 25° C., with gentle agitation, for 1 h. Blots were washed three times for 10 min with TBST (Tris-HCl pH 7.5, 150 mM NaCl, 0.1% Tween-20) and incubated at 25° C., with gentle agitation, for 1 h with the anti-mouse IgG HRP-conjugated antibody (diluted 1:1,000, clone 7076, Cell Signaling Technology). Blots were washed as described, developed using the ECL Plus substrate (Thermo Fisher) and imaged with X-ray films.

Assessment of the expression levels of Cbx7.VD-2x and Cbx2. VD-2x in mESC wild-type or mESC Eed-KO cells was performed by loading 10 μg of each WCL. Membranes were blocked for 1 h at 25° C. in TBSM and probed with anti-GFP rabbit polyclonal antibody (diluted 1:1,000, ab290, Abcam) and anti-Lamin B1 mouse antibody (diluted 1:2,000, sc-374015, Santa Cruz), in TBST buffer at 4° C., with gentle agitation, overnight. Following incubation, membranes were washed three times for 10 min with TBST and incubated at 25° C., with gentle agitation, for 1 h with anti-rabbit (NA934, Cytiva) or anti-mouse (NA931, Cytiva) IgG HRP-conjugated antibodies (both diluted 1:10,000), respectively. Blots were washed, developed, and imaged using a ChemiDoc Touch imaging system (BioRad). For analysis of ERK and ARPC2 repression, 20 μg of each WCL of cells stably expressing dCas9-fused repressors and gRNAs were transferred onto Immobilon-P PVDF membranes as described above.

Membranes were blocked for 1 h at 25° C. in TBSM and probed with the following antibodies: 0.5 μg/ml mouse anti-CRISPR-Cas9 (clone 7A9, EMD Millipore), rabbit anti-ARPC2 (diluted 1:1000, clone EPR8533, ab133315, Abcam), mouse anti-ERK1 (diluted 1:1000, clone G-8, sc-271269, Santa Cruz) and 0.44 μg/ml anti-GAPDH (clone FF26A, Abcam) in TBSM buffer containing 0.05% Tween-20 at 4° C., with gentle agitation, overnight. Following incubation with primary antibodies, membranes were washed three times for 10 min with TBST and incubated at 25° C., with gentle agitation, for 1 h with the anti-mouse IgG HRP conjugated antibody (diluted 1:1000, clone 7076, Cell Signaling Technology) or the anti-rabbit IgG HRP-conjugated antibody (diluted 1:1000, clone 7074, Cell Signaling Technology). Blots were washed, developed, and imaged using X-ray films, and band intensities were quantified by densitometry using the ImageJ software (version 1.53t). Percent relative expression was calculated as 100× (band intensity of untransduced cells/band intensity of samples stably expressing CRISPRi repressors and gRNAs).

### Example 2. High-Affinity Variants of the Cbx7 Chromodomain

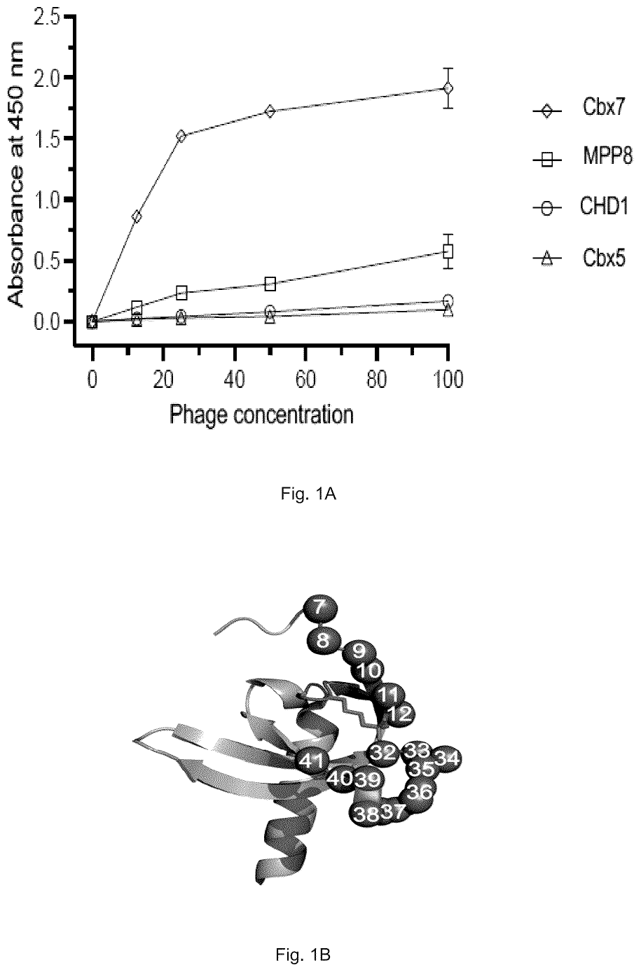

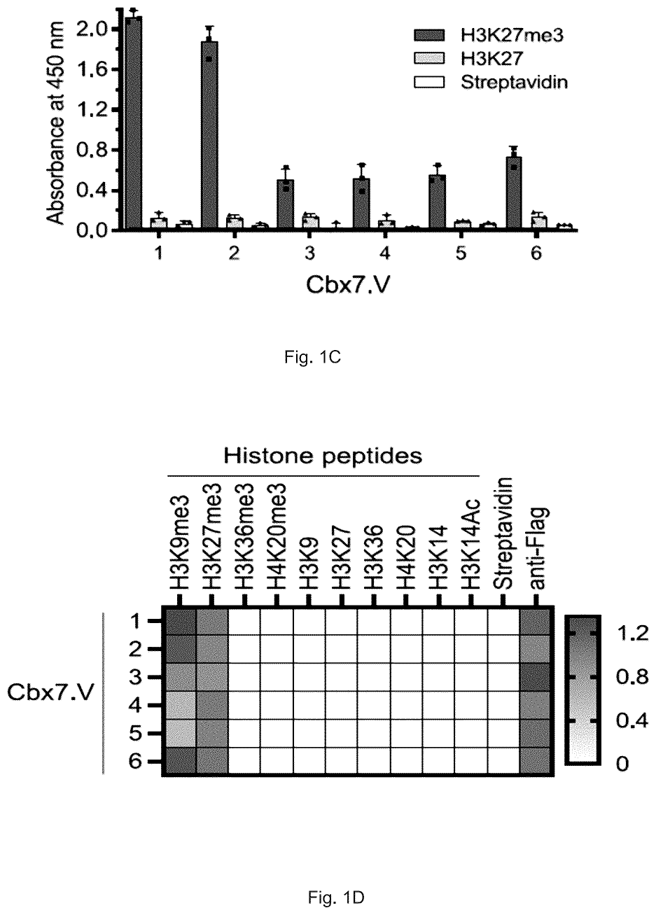

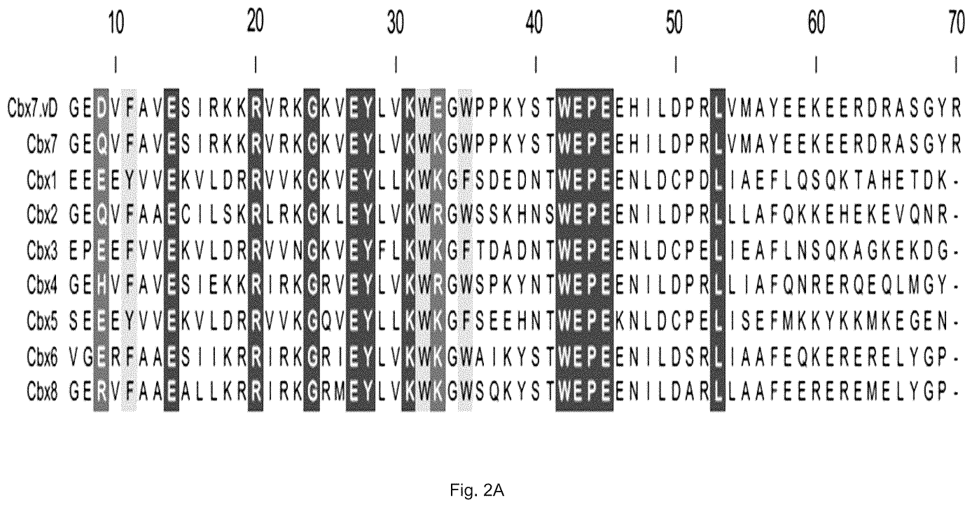

To develop a chromodomain variant with enhanced affinity for lysine methylation, the chromodomain of human Cbx7 (Cbx7-chromo) was selected over other human chromodomains for several reasons: (1) unlike Cbx1-chromo which is characterized.by high affinity for H3K9me3, but does not bind to H3K27me3 peptides, Cbx7-chromo exhibits low affinity for both the H3K9me3 and H3K27me3 marks (KD=55 or 110 μM, respectively) 26, (2) it lacks cysteine residues that can interfere with phage display, and (3) it showed strong display on phage in comparison with other chromodomains (FIG. 1A).

To aid library design, the structure of Cbx7-chromo in complex with a H3K27me3 peptide (PDB: 4X3K) was examined, and two continuous stretches that lined the methyllysine-binding pocket were identified for diversification, including 6 residues within the N-terminal β-strand (positions 7-12, region 1) and 10 residues on the other side of the pocket (positions 32-41, region 2; FIG. 1B). This residue set included three key amino acids that form the aromatic cage (Phe11, Trp32, and Trp35). These 16 positions were systematically subjected to a soft randomization mutagenesis strategy that favored the wild-type (wt) sequence but allowed ~50% mutations at each position, and a phage-displayed library was constructed containing 1.1×10¹⁰ unique Cbx7-chromo variants (Cbx7.Vs). The phage displayed Cbx7. V library was cycled through five rounds of binding selections with an immobilized H3K27me3 peptide, and a negative selection step was included to deplete clones that bound to the non-methylated version of the H3K27 peptide. The sequences of unique Cbx7 variants used in the study are listed in Table 9. DNA sequencing of selected clones revealed six unique Cbx7 variants (Cbx7.V1-6, Table 10), and most substitutions were found in seven positions (7, 8, 9, 12, 33, 37, and 38). The three positions that form the hydrophobic cage were conserved, except for a conservative F11Y substitution in one variant. Notably, the variants were all more negatively charged than the wild-type chromodomain, due to acidic substitutions in place of positively charged residues Lys33 and Lys38, and neutral residue Gln9. Phage ELISAs confirmed that all six variants bound to the H3K27me3 peptide, but not to the H3K27 peptide or streptavidin (FIG. 1C). The binding specificity of each Cbx7.V against a panel of tri-methylated and non-methylated histone peptides was assessed by phage ELISA. The sequences of peptides used in the study are listed in Table 1. The result shows that all the variants bound only to H3K9me3 and H3K27me3 peptides (FIG. 1D). Cbx7. V1 was further characterized, because it exhibited the strongest signal for the H3K27me3 peptide by phage ELISA and it did not contain any cysteine residues. Isothermal titration calorimetry (ITC) assays showed that Cbx7.V1 bound to H3K9me3 and H3K27me3 peptides with near 1:1 stoichiometry and affinities in the low micromolar range (KD=7.0 and 7.8 μM for H3K9me3 or H3K27me3, respectively. Table 10 discloses SEQ ID NOS 99-112, respectively, in order of appearance.

TABLE 9
Sequences of Selected Cbx7.Vs.

| SEQ ID NO | Cbx Vs. | Sequence |
|---|---|---|
| 27 | Cbx7 WT | SAIGEQVFAVESIRKKRVRKGKVEYLVKWKGWPPKYSTWE |
| | (095931, 4-75) | PEEHILDPRLVMAYEEKEERDRASGYRKRGPK |
| 92 | CBX7.V1 | SAIGDDIFAVESIRKKRVRKGKVEYLVKWEGWPLEYSTWE |
| | | PEEHILDPRLVMAYEEKEERDRASGYRKRGPK |
| 93 | CBX7.V2 | SAICDNVYPVESIRKKRVRKGKVEYLVKWKGWPLEYSTWE |
| | | PEEHILDPRLVMAYEEKEERDRASGYRKRGPK |
| 94 | CBX7.V3 | SAICEYIFSVESIRKKRVRKGKVEYLVKWEGWPPEYMTWEP |
| | | EEHILDPRLVMAYEEKEERDRASGYRKRGPK |
| 95 | CBX7.V4 | SAICDNVFAVESIRKKRVRKGKVEYLVKWEGWPQEYSTWE |
| | | PEEHILDPRLVMAYEEKEERDRASGYRKRGPK |
| 96 | CBX7.V5 | SAIGHDVFPVESIRKKRVRKGKVEYLVKWRDWPEQYSTWE |
| | | PEEHILDPRLVMAYEEKEERDRASGYRKRGPK |
| 97 | CBX7.V6 | SAIFEDVFPVESIRKKRVRKGKVEYLVKWKGWPSEYSTWE |
| | | PEEHILDPRLVMAYEEKEERDRASGYRKRGPK |

TABLE 10
Sequence Alignment of Selected Cbx7.Vs.

| | Region 1 | | | | | | Region 2 | | | | | | | | | |
|---|---|---|---|---|---|---|---|---|---|---|---|---|---|---|---|---|
| | 7 | 8 | 9 | 10 | 11 | 12 | 32 | 33 | 34 | 35 | 36 | 37 | 38 | 39 | 40 | 41 |
| | \| | \| | \| | \| | \| | \| | \| | \| | \| | \| | \| | \| | \| | \| | \| | \| |
| Cbx7.wt | G | E | Q | V | F | A | W | K | G | W | P | P | K | Y | S | T |
| Cbx7.V1 | — | D | D | I | — | — | — | E | — | — | — | L | E | — | — | — |
| Cbx7.V2 | C | D | N | — | Y | P | — | — | — | — | — | L | E | — | — | — |
| Cbx7.V3 | C | — | Y | I | — | S | — | E | — | — | — | — | E | — | M | — |
| Cbx7.V4 | C | D | N | — | — | — | — | E | — | — | — | Q | E | — | — | — |
| Cbx7.V5 | — | H | D | — | — | P | — | R | D | — | — | E | Q | — | — | — |
| Cbx7.V6 | F | — | D | — | — | P | — | — | — | — | — | S | E | — | — | — |

### Example 3 Site-Directed Mutagenesis of Cbx7.V1 And Cbx7.Wt

Cbx7.V1 contains six substitutions relative to the wild-type chromodomain, and to investigate how each substitution affected function, each of the six residues were mutated individually back to the wild-type sequence and affinities for the H3K9me3 and H3K27me3 peptides were measured using bio-layer interferometry (BLI, Table 11). Only two back-mutations resulted in large reductions in affinity: D9Q and E33K reduced affinity for the H3K9me3 or H3K27me3 peptide, respectively. These results showed that negatively charged substitutions were mostly responsible for enhanced affinity, but the E38K back-mutation resulted in moderate increases in affinities for both peptides.

TABLE 11
Dissociation constants for Cbx7.V1 and back-
mutants for methyllysine-containing peptides

| | K_{d} (μM) | |
|---|---|---|
| Domain | H3K9me3 | H3K27me3 |
| Cbx7.V1 | 4.5 ± 0.5 | 2.0 ± 0.1 |
| D8E | 5.0 ± 0.5 | 2.0 ± 0.2 |
| D9Q | >20 | 2 ± 1 |
| I10V | 3.5 ± 0.5 | 2.0 ± 0.2 |
| E33K | 10 ± 2 | >20 |
| L37P | 3.0 ± 0.5 | 2.5 ± 0.5 |
| E38K | 2.0 ± 0.1 | 1.5 ± 0.1 |

To further explore the effects of Asp9 and Glu33 residues in Cbx7.V1, these two substitutions were introduced either separately or together into wild-type Cbx7-chromo (Cbx7.wt) and the affinities for the H3K9me3 and H3K27me3 peptides were measured by fluorescence polarization (FP) (Table 12). Both single substitutions improved affinities for both peptides, and consistent with the back-mutation analysis of Cbx7.V1 (Table 3), the Q9D or K33E substitution had the greatest effect on affinity for the H3K9me3 or H3K27me3 peptide, respectively. The variant containing the double Q9D/K33E substitutions (Cbx7.VD) exhibited higher affinity for both peptides compared with the variants with single substitutions, and these affinities were virtually identical to those of Cbx7.V1. Importantly, Cbx7.VD retained specificity for methyllysine, as it did not bind to unmethylated H3K9 or H3K27 peptides. Taken together, these data confirmed that, amongst the six substitutions in Cbx7.V1, the two substitutions Q9D/K33E are the only ones that contribute significantly to affinity enhancement for methylated histone peptides.

TABLE 12
Dissociation constants for Cbx7.wt and variants
for methylated histone peptides

| | K_{d} (μM) | |
|---|---|---|
| Domain | H3K9me3 | H3K27me3 |
| Cbx7.wt | 30 ± 10 | 110 ± 40 |
| Cbx7 Q9D | 10 ± 1 | 100 ± 20 |
| Cbx7 K33E | 20 ± 3 | 20 ± 2 |
| Cbx7.V1 | 7.0 ± 1.5 | 4.0 ± 0.5 |
| Cbx7.VD | 8.0 ± 0.5 | 5.0 ± 0.2 |

### Example 4 Effects of the Asp9/Glu33 Double-Substitution Across the Human Cbx Chromodomain Family

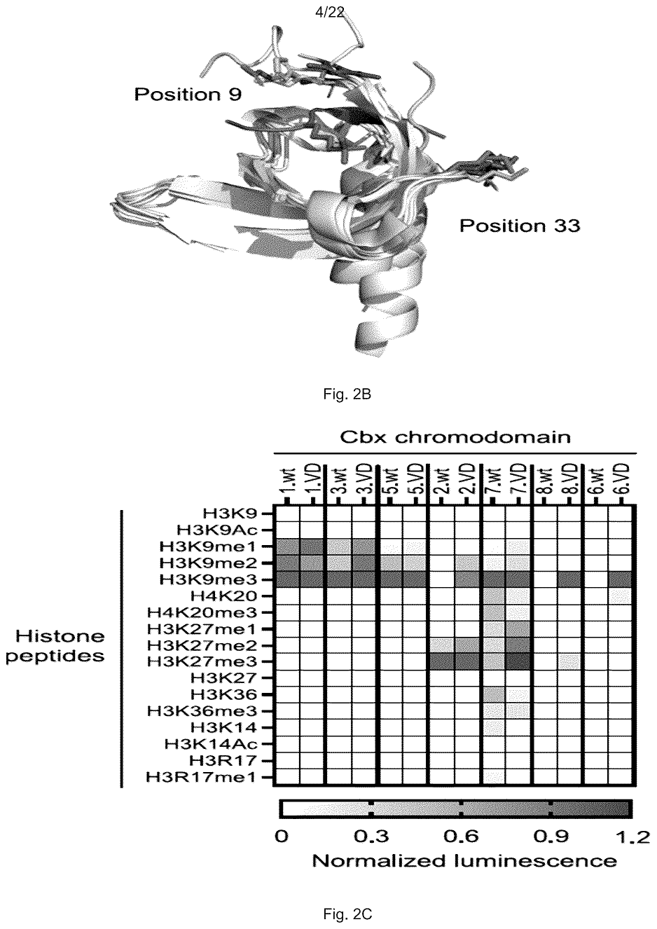

Next, to confirm whether the Asp9/Glu33 double-substitution could enhance the functions of other chromodomains from the Polycomb group (PcG) of proteins (Cbx2, Cbx4, Cbx6, Cbx7, and Cbx8) and the heterochromatin protein 1 (HP1) family members (Cbx1, Cbx3, and Cbx5), The primary sequence of each chromodomain was investigated and a conserved positively charged residue at position 33 was observed (FIG. 2A). While all PcG protein members contained polar or positively charged residues at position 9 (except for Cbx6, which contained a Glu), all HP1 family members were characterized by a Glu residue at the same position. Next, the structures of all Cbx chromodomains with that of Cbx7-chromo in complex with an H3K27me3 peptide were superposed and the location of residues at positions 9 and 33 were mapped (FIG. 2B). The other structures superposed well with that of Cbx7-chromo (RMSD=0.5-2.0 Å), and in all cases, the side chains at positions 9 and 33 aligned well with those of Cbx7-chromo. Thus, given the high structural similarity and high sequence homology of Cbx chromodomains, the Asp9/Glu33 substitutions may be able to enhance the affinity of the other Cbx chromodomains for methylated histone peptides.

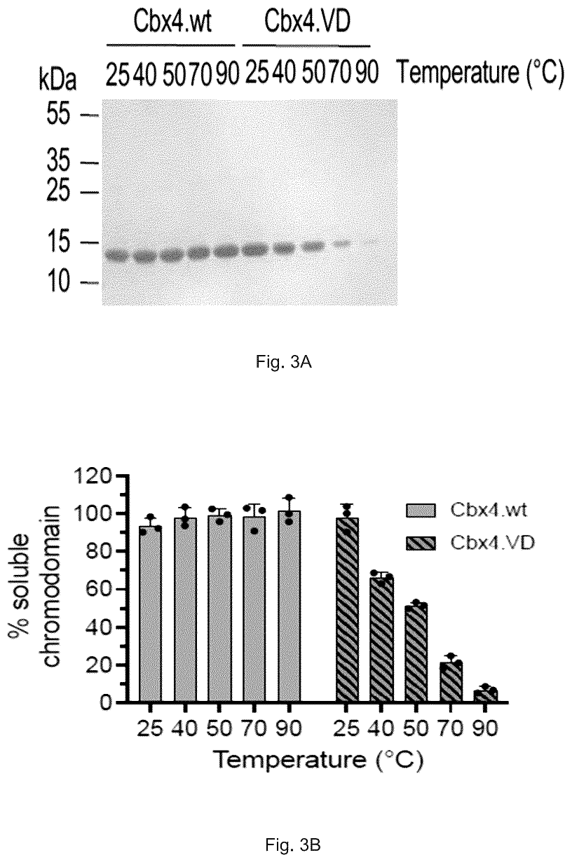

To test this hypothesis, residues at positions 9 and 33 were substituted with Asp or Glu, respectively, in all Cbx chromodomains (Table 3 and Table 4) and a “Cbx. VD” panel (“VD” was assembled to indicate the Asp9/Glu33 double-substitution). FP assays was used to measure the affinities of each wild-type Cbx chromodomain (Cbx.wt) and its Cbx. VD version for the H3K9me3 and H3K27me3 peptides (Table 13). In the case of Cbx4-chromo, the substitutions had a detrimental effect, and they had no effect in the case of Cbx1-chromo, which already bound to the H3K9me3 peptide with sub-micromolar affinity. Since the substitutions had a negative effect on Cbx4-chromo, the thermal stability of both Cbx4.wt and Cbx4. VD was assessed, and it was found that the substitutions destabilized the domain (FIGS. 3A-3B). In all other cases, affinities were improved significantly. Like Cbx1-chromo, the chromodomains of Cbx3 and Cbx5 bound only the H3K9me3 peptide, and in both cases, the substitutions improved binding for this peptide but did not enhance binding to the H3K27me3 peptide. Like Cbx7-chromo, the chromodomains of Cbx2 and Cbx8 bound weakly to both peptides, and in both these cases, the substitutions dramatically improved binding to both peptides, as was the case for Cbx7-chromo. Finally, Cbx6-chromo did not bind appreciably to either peptide, but the Asp9/Glu33 double-substitution did enable binding to the H3K9me3 peptide.

TABLE 13
Dissociation constants for Cbx.wt and Cbx.VD
pairs for methyllysine-containing peptides

| | K_{d} (μM) | |
|---|---|---|
| Domain | H3K9me3 | H3K27me3 |
| Cbx4.wt | 80 ± 10 | >200 |
| Cbx4.VD | NB | NB |
| Cbx1.wt | 0.5 ± 0.1 | NB |
| Cbx1.VD | 0.6 ± 0.1 | NB |
| Cbx3.wt | 2 ± 1 | NB |
| Cbx3.VD | 0.35 ± 0.1 | NB |
| Cbx5.wt | 5.0 ± 0.5 | NB |
| Cbx5.VD | 0.5 ± 0.1 | NB |
| Cbx2.wt | >200 | 100 ± 20 |
| Cbx2.VD | 5 ± 1 | 3.0 ± 0.2 |
| Cbx7.wt | 30 ± 10 | 110 ± 40 |
| Cbx7.VD | 8.0 ± 0.5 | 5.0 ± 0.2 |
| Cbx8.wt | >200 | >200 |
| Cbx8.VD | 20 ± 5 | >50 |
| Cbx6.wt | NB | NB |
| Cbx6.VD | >50 | NB |

To gain structural insights about the effect of Q9D/K33E, AlphaFold230 was used to model the three-dimensional structure of each Cbx. VD. Cbx1, 3, and 5 are characterized by a large electronegative histone peptide interacting surface, whereas PcG chromodomains display a more hydrophobic surface. The results show the Q9D/K33E substitutions increased the electronegative potential of the peptide-binding surface, and therefore, may establish favorable interactions with the positively charged histone peptides.

Next, specificity was assessed in greater detail by measuring binding of each wild-type chromodomain (except Cbx4) and its variant across a panel of histone peptides. To enhance detection sensitivity, each chromodomain was expressed as a fusion to the N-terminus of an engineered firefly luciferase and binding was measured by luminescence intensity. In all cases, the results (FIG. 2C) were consistent with the results of the FP assays (Table 13), and binding was strongest for peptides containing trimethylated lysine residues. The chromodomains of Cbx1, Cbx3 and Cbx5 bound only to peptides that were methylated at the H3K9 position, and in the case of Cbx3, binding was significantly stronger for the engineered variant compared with the wild-type chromodomain. In contrast, the chromodomains of Cbx2, Cbx7, and Cbx8 bound to peptides that were methylated at either the H3K9 or H3K27 position, and in every case, the variants bound more strongly than the wild type. Indeed, Cbx8.wt did not bind detectably to any peptides and Cbx2.wt did not bind detectably to peptides methylated at position H3K9, but both variants (Cbx2.VD and Cbx8.VD) exhibited robust and selective binding. Notably, Cbx7.wt and Cbx7.VD exhibited binding to several additional peptides, but Cbx7.VD bound most strongly to the H3K9me3 and H3K27me3 peptides. Finally, Cbx6.wt did not bind to any peptides, whereas Cbx6.VD exhibited strong binding to the H3K9me3 peptide and weak binding to the H4K20 peptide, and this interaction was observed previously for Cbx6.wt.

Taken together, these results showed that the Asp9/Glu33 double substitution provides a general means for enhancing the affinities of most chromodomains of the Cbx family, without altering their specificity significantly.

### Example 5. Functional Characterization of High-Affinity Chromodomains in mESCs

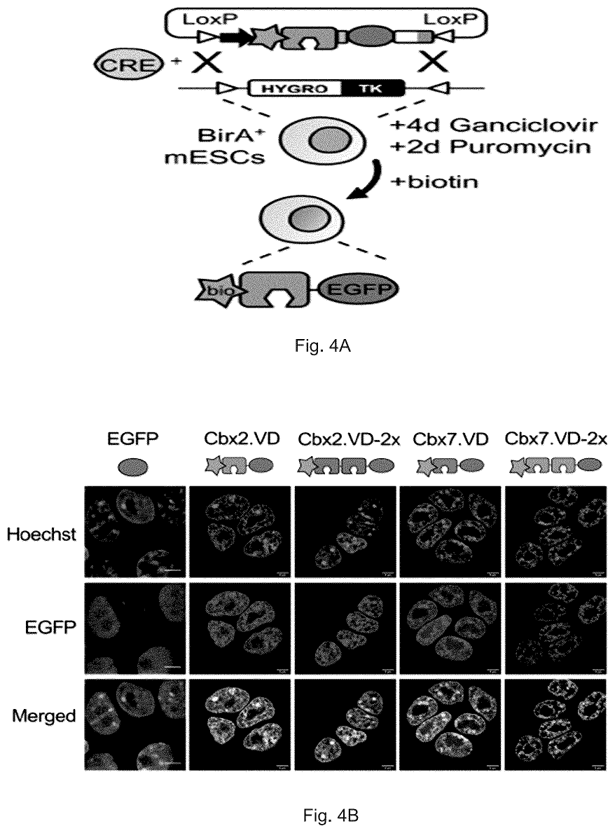

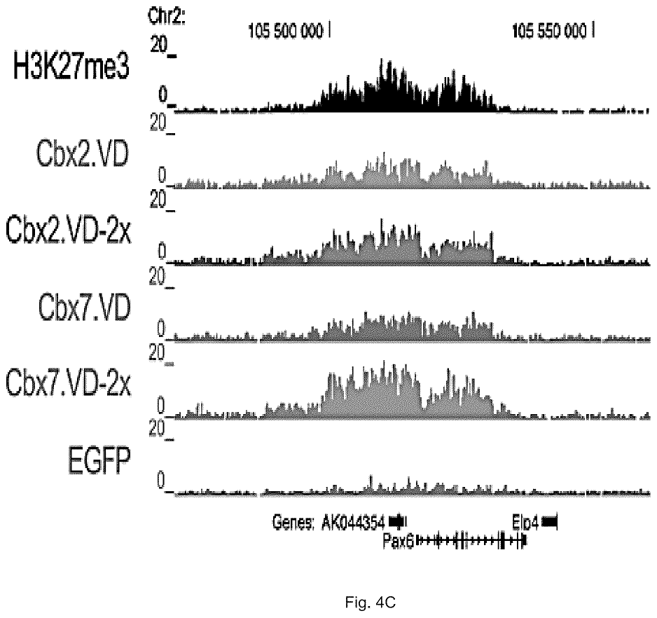

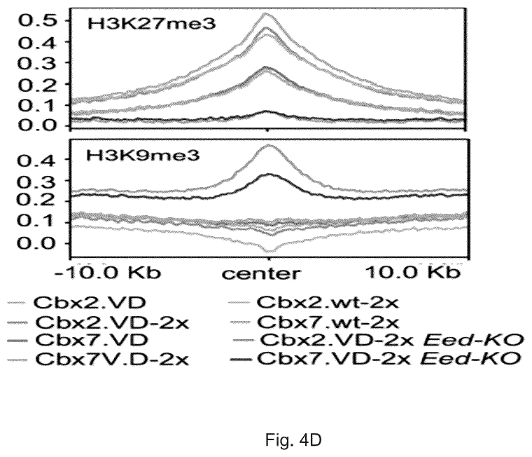

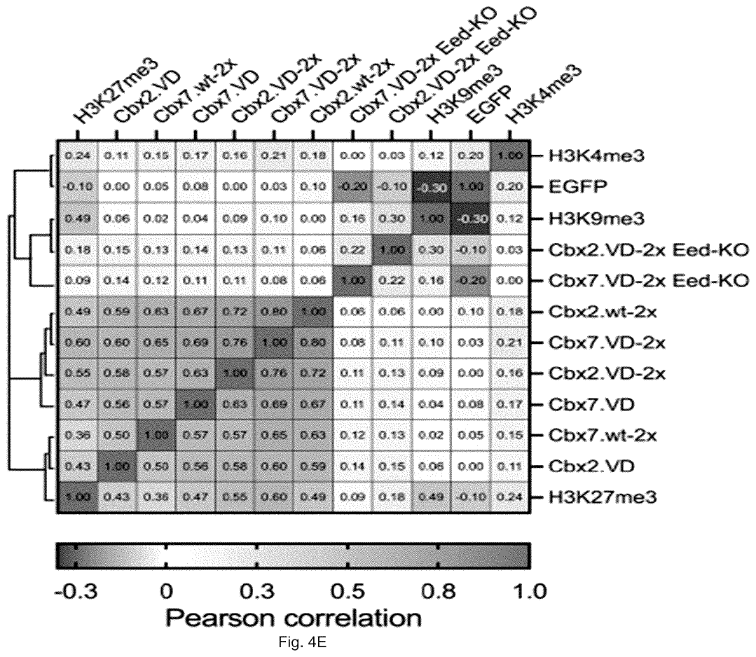

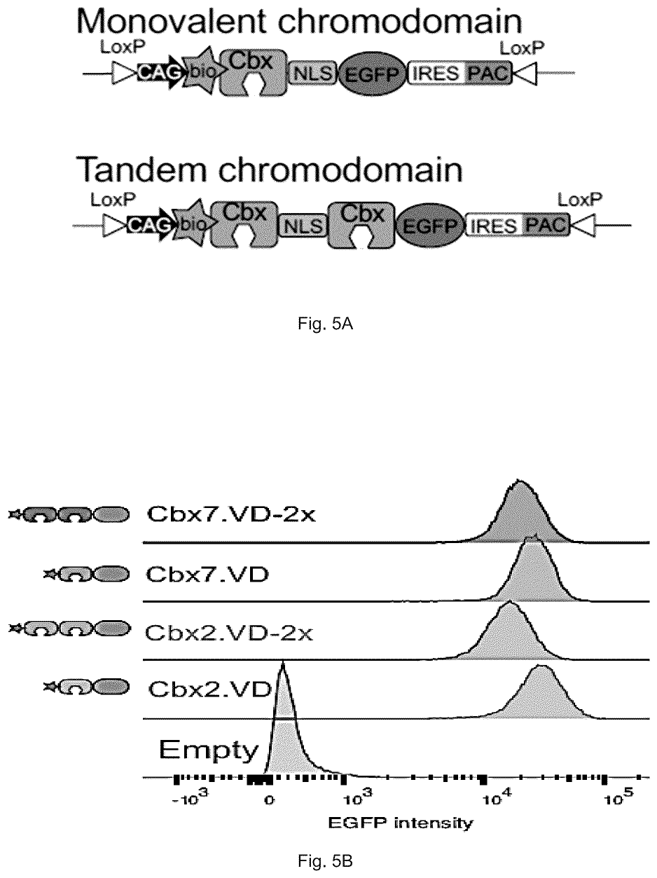

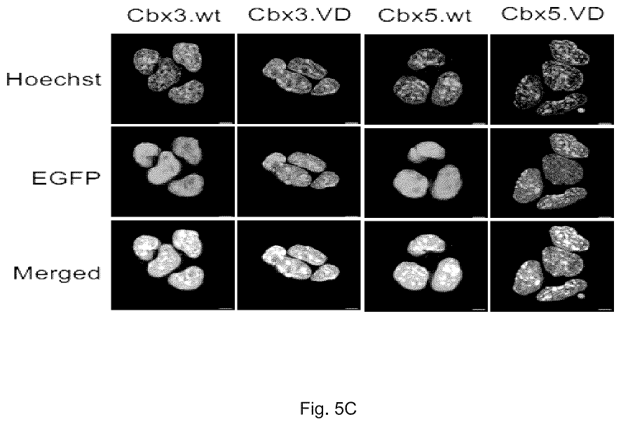

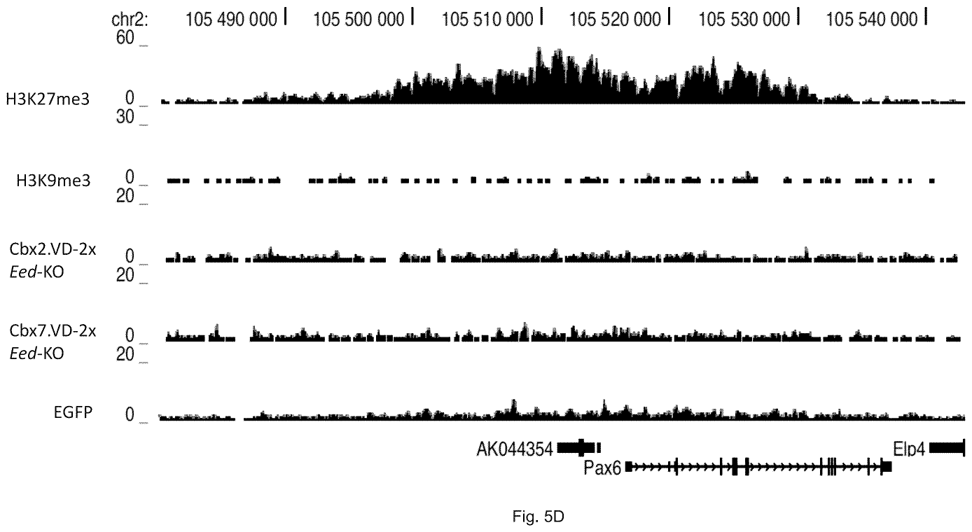

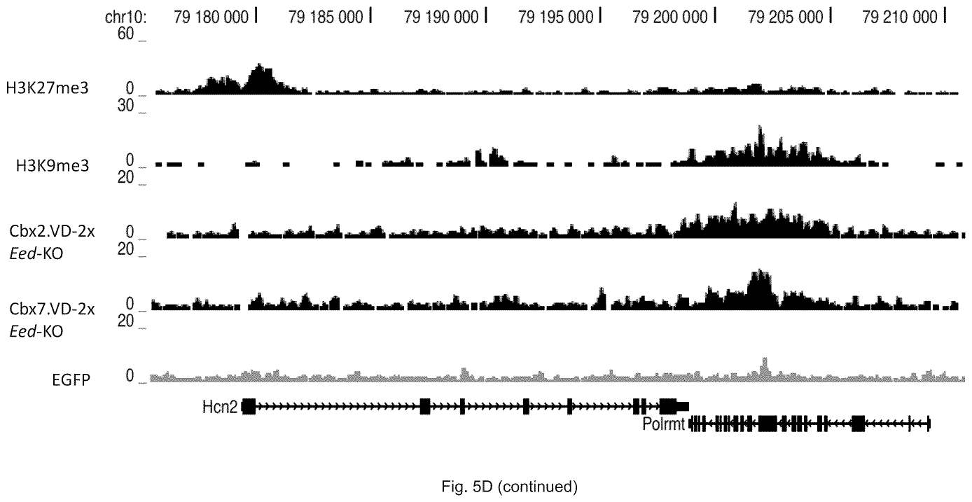

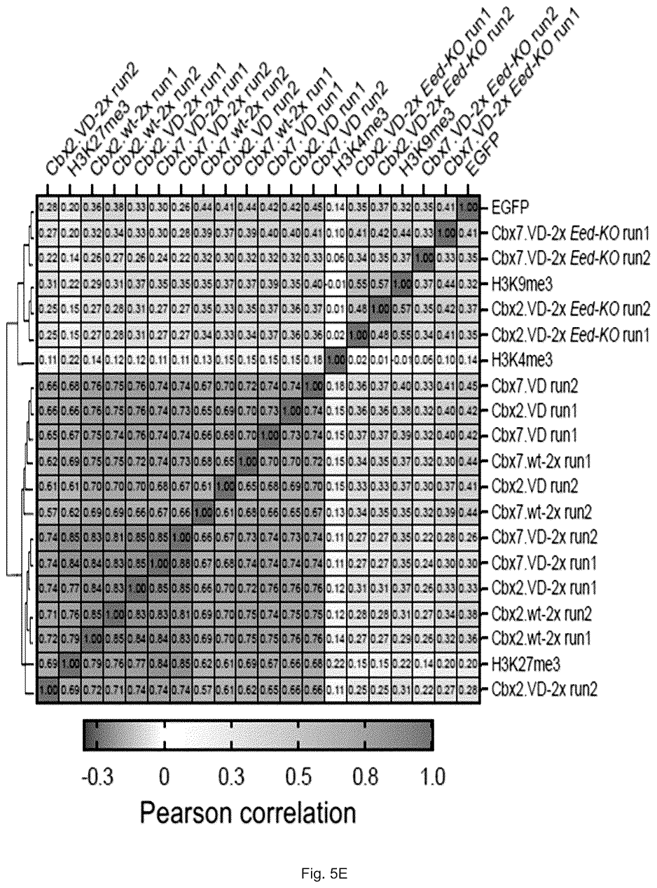

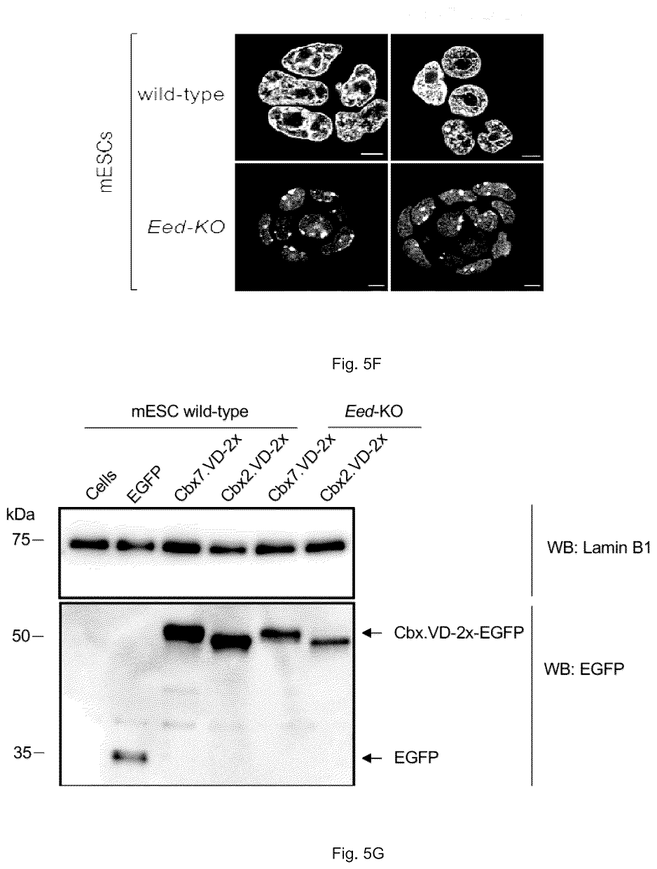

To explore activities of the high-affinity chromodomains in live-cells, Cbx3.wt, Cbx3.VD, Cbx5.wt, Cbx5.VD, Cbx7.VD, or Cbx2.VD were integrated into a defined site in the genome of mouse embryonic stem cells (mESCs) by recombinase-mediated cassette exchange, in either a single-domain or tandem double-domain configuration (Cbx.VD-2x; FIG. 5A). Each domain was fused to an N-terminal biotin acceptor site to allow immunoprecipitation, and at the C-terminus, to a nuclear localization signal (NLS) followed by EGFP to enable live-cell imaging (FIGS. 4A, FIG. 5A). The sequences of the constructs are listed in Table 5. Flow cytometry analysis of cell lines showed homogeneous and stable expression of each biotin-Cbx-EGFP fusion (FIG. 5B). Live-cell imaging of mESCs expressing Cbx3.VD, Cbx5.VD, Cbx7.VD or Cbx2.VD showed punctate nuclear localization with accumulation at chromocenters, the nuclear periphery, and around nucleoli, in contrast to the diffuse pattern observed for EGFP alone (FIG. 4B and FIG. 5C). Strikingly, this localization pattern of the high affinity single-domain proteins was similar to what was previously observed for Cbx7.wt arranged in the tandem double-domain configuration (Cbx7.wt-2x), whereas the single-domain Cbx7.wt did not exhibit specific nuclear localization. Lastly, cells expressing high affinity double-domain proteins (Cbx2. VD-2x and Cbx7.VD-2x) displayed even more discrete punctate staining, with both engineered chromodomains accumulating at the nuclear periphery and around nucleoli (FIG. 4B).

The genome-wide binding patterns of the high affinity chromodomains was measured using biotin-mediated chromatin immunoprecipitation followed by sequencing (biotin ChIP-seq) 32. Genomewide binding analyses revealed specific enrichment at regions marked with H3K27me3 for high-affinity single-domain and double-domain proteins (FIGS. 4C-4D). Notably, Cbx2.VD and Cbx7.VD displayed similar enrichments at H3K27me3 sites compared to our previously engineered H3K27me3 chromatin reader containing double-domain Cbx7.wt-2x, but both were significantly outperformed by their own double-domain versions (Cbx2. VD-2x and Cbx7.VD-2x). These results highlight the importance of multivalent interactions for efficient histone-mark recognition in cells (FIG. 4C).

These interactions were specific, as observed from genome-wide association of both Cbx2.VD-2x and Cbx7.VD-2x to the same histone modifications recognized by the wild-type chromodomains (FIG. 4E). No detectable binding signals for high-affinity single domain or double-domain proteins at H3K9me3 sites in wild-type mESCs were observed (FIG. 4D bottom and FIGS. 5D-5E). However, when Cbx2.VD-2x or Cbx7.VD-2x were expressed in cells that lacked H3K27 methylation (Eed-KO cells), each domain displayed loss of binding to H3K27me3 regions but gained the ability to specifically interact with H3K9me3 sites, such as chromocenters (FIG. 4D bottom and FIGS. 5C-5F). These results showed that, despite their ability to interact with both H3K9me3 and H3K27me3 marks in vitro, the chromodomains of Cbx2 and Cbx7 preferably recognized H3K27me3 modified histones in vivo independently of their expression levels (FIG. 5G). In summary, these results showed that engineered high-affinity chromodomains display strong binding to H3K27me3 marks in living cells, while maintaining the binding specificities of their wild-type counterparts.

### Example 6. Effects of High-Affinity Chromodomains on CRISPRi Efficiency

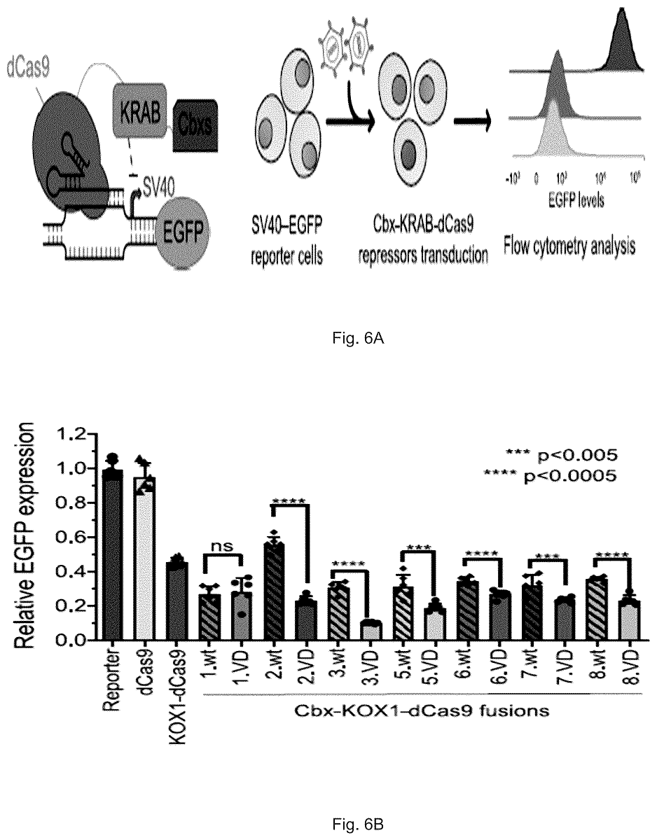

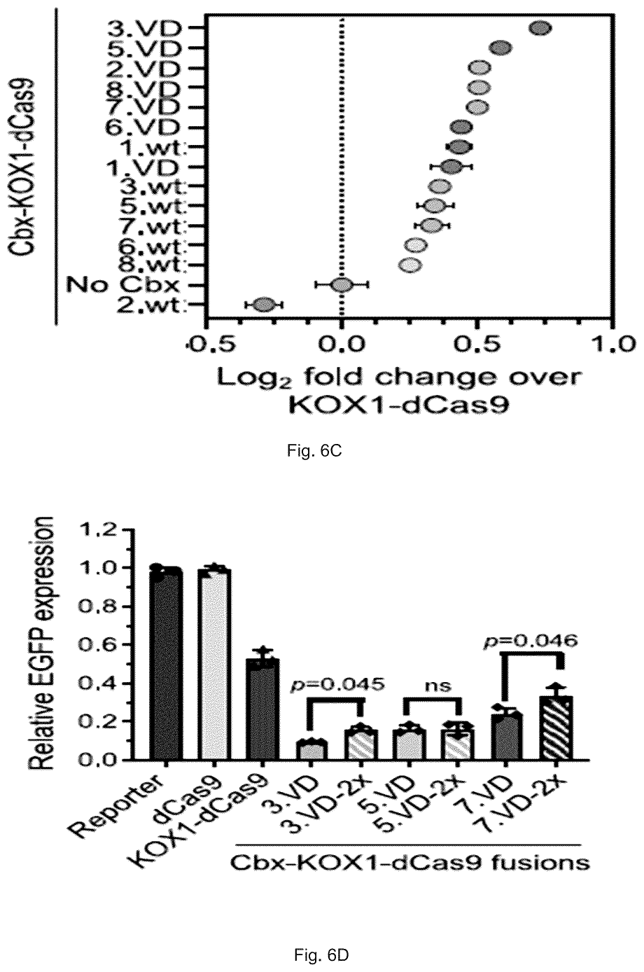

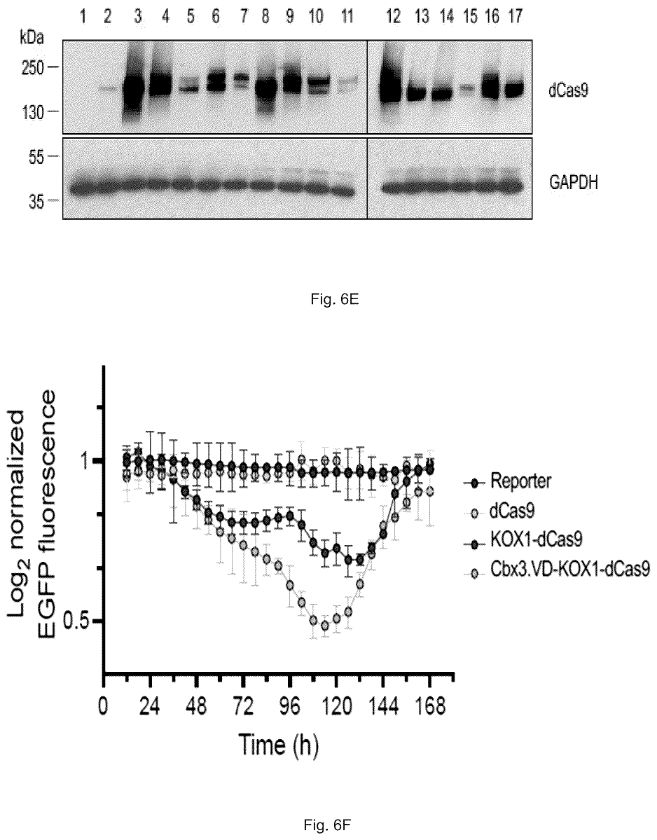

To make an enhanced CRISPRi repressor, each Cbx chromodomain (except Cbx4) and its high affinity variant was individually fused to the N-terminus of the KRAB domain of KOX1 fused to dCas9, and their ability to silence EGFP expression in the HEK293T pSV40-EGFP reporter cell line37 (FIG. 4A) was assessed. The sequences of the fused proteins are listed in Table 6. Cbx-KOX1-dCas9 fusions were delivered by lentiviral infection into HEK293T cells stably expressing EGFP froma SV40 promoter, and EGFP expression was measured by flow cytometry (FIGS. 6A-6B). All repressors decreased EGFP expression in comparison to reporter-only cells or reporter cells transduced with dCas9 only. The KRAB domain of KOX1 reduced expression of EGFP by ~50%, whereas, almost all Cbx-KOX1-dCas9 fusions were more potent. Except for Cbx2.wt, Cbx.wt-KOX1-dCas9 fusions reduced the expression of EGFP by 65% on average, with no significant differences between the domains. Notably, Cbx. VD-KOX1-dCas9 fusions containing high-affinity chromodomains enabled stronger repression of EGFP expression, and remarkably, the fusion containing Cbx3.VD reduced EGFP levels by ~90%. The extent of repression correlated strongly with the affinity of the domains for H3K9me3 and H3K27me3 marks. For example, while a marked difference in repression by Cbx1.wt was observed compared with Cbx1.VD, in agreement with their nearly identical affinities for H3K9me3 peptides (Table 13), Cbx2.VD provided 76% repression, whereas the lower affinity Cbx2.wt reduced EGFP expression by only 44%. Repression differences were not dependent on the expression levels of the different repressors, as western blot analysis showed that even poorly expressed repressors could strongly reduce EGFP levels (FIG. 6E). The repression efficiency of each chromodomain fusion was compared to that of KOX1-dCas9 and the comparison shows that the high-affinity chromodomain fusions showed on average a 40% improvement in repression (FIG. 6C). Most impressively, the Cbx3. VD fusion displayed 66% greater repression than KOX1-dCas9 and was 10% better than the second-best repressor containing the Cbx5. VD fusion.

To further evaluate the correlation between the affinity of chromodomains and their repressive activity, double-domain versions of Cbx-KOX1-dCas9 fusions containing two copies of Cbx3.VD, Cbx5.VD, or Cbx7.VD arranged in tandem were delivered by lentiviral infection and their repressive potential to single-domain high affinity chromodomain fusions were compared (FIG. 6D). Similarly, fusion of chromodomains to KOX1-dCas9 significantly reduced the expression levels of EGFP, but in general, each double-domain fusion performed worse than its single-domain counterpart. Cbx3.VD-KOX1-dCas9 was the strongest repressor, and it was highly potent even when transiently transfected into HEK293T SV40-EGFP reporter cells (FIG. 6F).

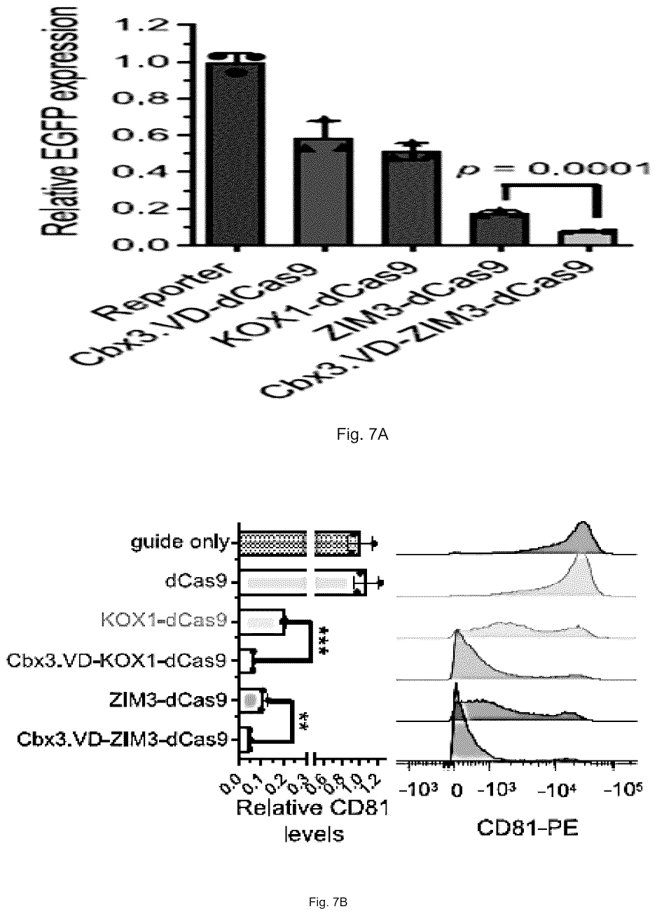

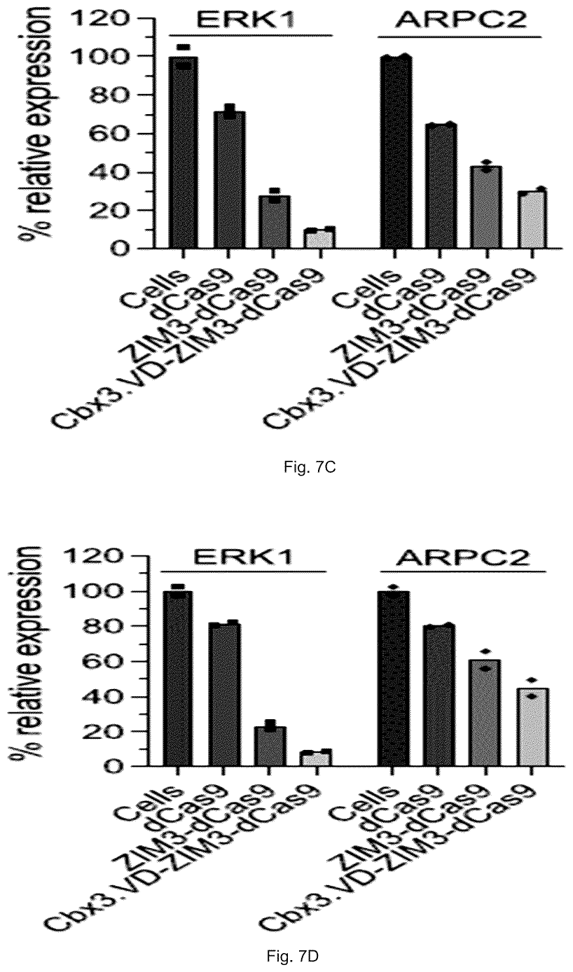

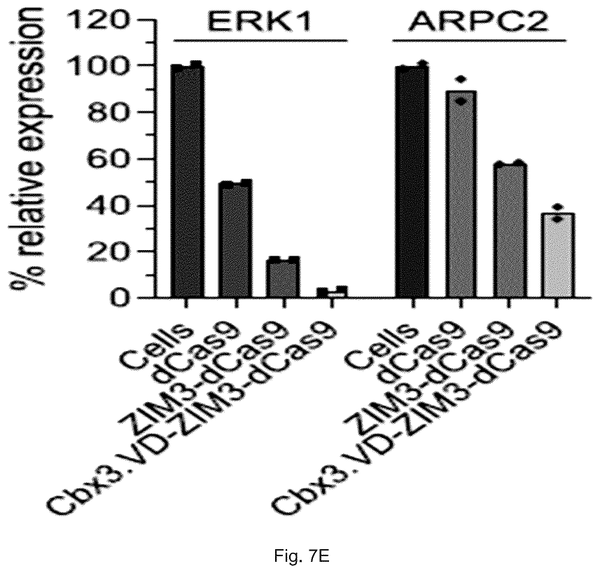

To evaluate the generality of the method for developing potent CRISPR-based repressors, Cbx3. VD was combined with the KRAB domain of ZIM3, which showed the strongest repressive potency among currently used CRISPRi repressors (FIG. 7A). A Cbx3.VD-dCas9 fusion (lacking the KRAB domain) reduced EGFP expression to levels comparable to the KOX1-dCas9 fusion, whereas a fusion of dCas9 to the ZIM3 KRAB domain (ZIM3-dCas9) had a stronger repressive effect on EGFP levels. Most notably, fusion of both Cbx3. VD and the ZIM3 KRAB domain to dCas9 (Cbx3.VD-ZIM3-dCas9) further improved repressive potency and reduced EGFP levels by 92%.

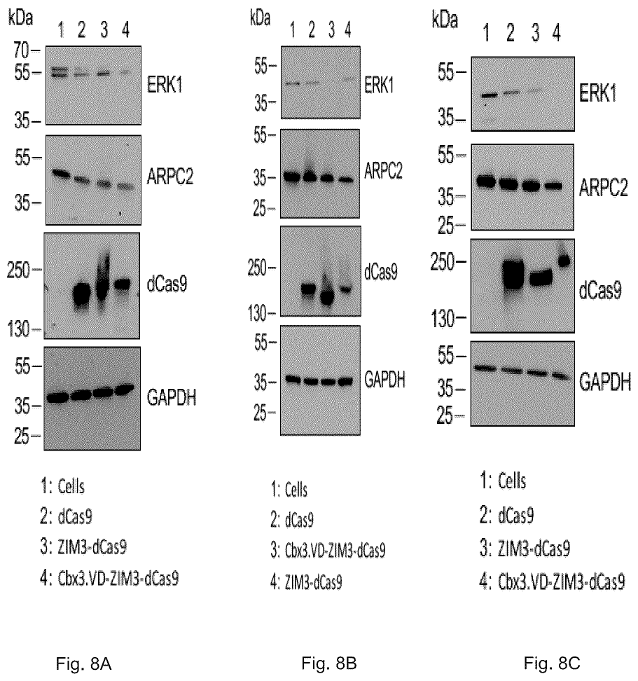

Lastly, to assess whether Cbx3.VD-KRAB domain fusions could outperform current versions of CRISPRi for repression of endogenous genes. repressors to the promoter of CD81 by lentiviral infection of HEK293T cells were recruited and gene silencing was assayed by flow cytometry. CRISPRi repressors significantly silenced CD81 expression, and strikingly, the additional fusion of Cbx3.VD to either the KRAB domain of KOX1 or ZIM3 greatly enhanced repression and nearly abolished cell surface levels of the CD81 protein (FIG. 7B). The robustness of the Cbx3. VD-ZIM3-dCas9 repressor was further validated by targeting two additional genes (ERK1 and ARPC2) in three different cell lines (HEK293T, HeLa and U20S) (FIGS. 7C-7E and FIGS. 8A-8C). Cbx3.VD-ZIM3-dCas9 displayed efficient repression of targeted genes in each of the tested cell lines, and improved gene silencing, relative to ZIM3-dCas9, in every case except for ARPC2 in U20S cells (FIG. 7D). Taken together, these results showed that the engineered high affinity chromodomains can be used to enhance gene repression in CRISPRi applications beyond what is possible with the best methods available currently.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An engineered chromodomain comprising:
a methyllysine-binding pocket, wherein the engineered chromodomain comprises at least one amino acid substitution in at least one of two regions that line the methyllysine-binding pocket as compared to a reference chromodomain selected from any one of SEQ ID NOS: 21-28, wherein a first region comprises amino acid positions 8-28 of the reference chromodomain, wherein a second region comprises amino acid positions 33-52 of the reference chromodomain, wherein the engineered chromodomain provides for enhanced binding to at least one methylated protein as compared to said reference chromodomain, wherein the at least one amino acid substitution comprises substitution of a neutral or positively charged amino acid residue to a negatively charged amino acid residue.

2. The engineered chromodomain of claim 1, wherein the at least one amino acid substitution comprises substitution of Gln to Asp or Glu.

3. The engineered chromodomain of claim 1, wherein the at least one amino acid substitution comprises substitution of Lys or Arg to Asp or Glu.

4. The engineered chromodomain of claim 1, wherein the at least one amino acid substitution comprises substitution of Gln to Asp and substitution of Lys to Glu.

5. The engineered chromodomain of claim 1, wherein the engineered chromodomain binds to a methylated protein with a dissociation constant (Kd) of 100 μM or less.

6. The engineered chromodomain of claim 1, wherein the engineered chromodomain binds to a methylated protein with a dissociation constant (Kd) of 50 μM or less.

7. The engineered chromodomain of claim 1, wherein the engineered chromodomain binds to a methylated protein with a dissociation constant (Kd) of 10 μM or less.

8. The engineered chromodomain of claim 1, wherein the reference chromodomain is selected from a group consisting of Cbx1, Cbx2, Cbx3, Cbx4, Cbx5, Cbx6, Cbx7 and Cbx8.

9. The engineered chromodomain of claim 1, wherein the methylated protein is a histone H3 protein.

10. The engineered chromodomain of claim 1, wherein the amino acid substitutions provide for an enhanced binding to a methylated histone H3 protein at position 9 (H3K9), (H3K27), or both.

11. The engineered protein chromodomain of claim 10, where the methylated H3 protein is monomethylated, dimethylated, or trimethylated.

12. The engineered chromodomain of claim 1, wherein the engineered chromodomain demonstrates enhanced binding to H3K27me3, H3K9me3, or both compared to the reference chromodomain under comparable conditions.

13. A kit comprising the engineered chromodomain of claim 1.

14. A composition comprising an engineered chromodomain comprising:
a methyllysine-binding pocket, wherein the engineered chromodomain comprises at least one amino acid substitution in at least one of two regions that line the methyllysine-binding pocket as compared to a reference chromodomain selected from any one of SEQ ID NOS: 21-28, wherein a first region comprises amino acid positions 8-28 of the reference chromodomain, wherein a second region comprises amino acid positions 33-52 of the reference chromodomain, wherein the engineered chromodomain provides for enhanced binding to at least one methylated protein as compared to said reference chromodomain, wherein the at least one amino acid substitution comprises substitution of a neutral or positively charged amino acid residue to a negatively charged amino acid residue; and
a buffer.

15. The composition of claim 14, wherein the engineered chromodomain comprises an amino acid sequence having at least 99% sequence identity to an amino acid sequence selected from SEQ ID NOS: 29-30 or SEQ ID NOS: 32-36, or at least 95% sequence identity to the amino acid sequence of SEQ ID NO: 31.

16. The composition of claim 14, further comprising at least one detectable label.

17. The composition of claim 16, wherein the detectable label is a radioactive label, a biotin-based label, a fluorescent label, an electron-dense reagent, or an enzyme.

18. The composition of claim 17, wherein the fluorescent label is fluorescein, rhodamine, Texas Red, or a fluorescent protein.

19. The composition of claim 17, wherein the enzyme is alkaline phosphatase, horseradish peroxidase, or luciferase.

20. The composition of claim 14, further comprising a nuclear localization signal (NLS).

21. The composition of claim 14, wherein the buffer comprises a citrate, phosphate, succinate, tartrate, fumarate, gluconate, oxalate, lactate, acetate, histidine, or tris buffer.

22. A nucleic acid, wherein the nucleic acid encodes for the engineered chromodomain of claim 1.

23. A kit comprising an engineered chromodomain comprising an amino acid sequence having at least 99% sequence identity to an amino acid sequence selected from SEQ ID NOS: 29-30 or SEQ ID NOS: 32-36, or at least 95% sequence identity to the amino acid sequence of SEQ ID NO: 31.

24. A method of detecting presence of a methylated protein in a biological sample comprising:
(a) providing a biological sample;
(b) contacting the biological sample with an engineered chromodomain that comprises a methyllysine-binding pocket, wherein the engineered chromodomain comprises at least one amino acid substitution in at least one of two regions that line the methyllysine-binding pocket as compared to a reference chromodomain selected from any one of SEQ ID NOS: 21-28, wherein a first region comprises amino acid positions 8-28 of the reference chromodomain, wherein a second region comprises amino acid positions 33-52 of the reference chromodomain, wherein the engineered chromodomain provides for enhanced binding to at least one methylated protein as compared to said reference chromodomain, wherein the at least one amino acid substitution comprises substitution of a neutral or positively charged amino acid residue to a negatively charged amino acid residue; and
(c) detecting interaction of the biological sample with the engineered chromodomain, wherein the interaction indicates the presence of a methylated protein in the biological sample.
